(19) United States
(12) Patent Application Publication
(10) Pub. No.: US 2005/0130221 A1
(43) Pub. Date: Jun. 16, 2005

(54) Synthesis for the preparation of compounds for screening as potential tubulin binding agents

(75) Inventors: Bernard Luke Flynn, Hughes (AU); Ernest Hamel, Bethesda, MD (US)

Correspondence Address: BACON & THOMAS, PLLC, 625 SLATERS LANE, FOURTH FLOOR, ALEXANDRIA, VA, 22314, US

(73) Assignees: The Australian National University, Acton (AU); The Government of the United States as Represented by The Secretary of the Dept. of Health and Human, Rockville, MD (US)

(21) Appl. No.: 10/466,769

(22) PCT Filed: Feb. 1, 2002
(86) PCT No.: PCT/AU02/00099

(30) Foreign Application Priority Data
Feb. 1, 2001 (AU) PR 2838

(51) Int. Cl. G01N 33/53; C07D 333/74; C07F 9/547; G01N 33/543

(52) U.S. Cl. 435/7.1; 436/518; 548/412; 548/484; 549/44; 549/51

(57) ABSTRACT

The present invention relates to methods for the synthesis of chemical compounds for screening as potential tubulin polymerization inhibitors. The invention also provides chemical compounds with tubulin polymerization inhibitor activity.

### FIELD OF THE INVENTION

The present invention relates generally to chemical compounds and methods for their preparation. In particular, the invention relates to chemical compounds, and libraries thereof, which may possess useful therapeutic activity, and their use in methods of therapy as well as compositions containing said compounds.

### BACKGROUND OF THE INVENTION

The search for effective chemotherapeutic drugs relies on the discovery and development of chemical compounds which possess biologically potent anti-tumour activities. While many naturally occurring compounds have been recognised as possessing this desirable activity, eg taxol, the inherent difficulties which may be associated with the isolation and purification of naturally occurring small quantities have prompted extensive efforts directed towards the chemical synthesis of analogues of these bioactive compounds and other potentially bioactive molecules.

Compound libraries prepared by combinatorial methods now play an increasing role in the discovery of bioactive molecules. These libraries can be subjected to high throughput screening methods which allow for the rapid identification of potential new drug candidates. More recently, such libraries have also found utility in molecular biology as an aid to understanding various biological pathways. To reduce the cost of drug discovery using these methods, efficient means of producing molecular diversity need to be identified. Traditionally, efficiency in synthesis has been defined as providing the maximum yield of a targeted product. In diversity-orientated synthesis, efficiently is redefined as providing the maximum range of different structural entities from the minimum number of starting components.

One class of compounds which have attracted attention are those which inhibit tubulin assembly and prevent its polymerization into microtubules. Compounds with tubulin binding activity are thus effective anti-mitotic agents and include colchicine, vinblastine, vincristine and taxol. Another tubulin polymerization inhibitor which has attracted recent interest is Combretastatin A4 (A) which is a powerful inhibitor of tubulin polymerization with an IC₅₀ value of ˜2-3 μM and which has been shown to display potent and selective toxicity toward tumour vasculature. Its 3′-hydroxy disodium phosphate ester is currently the subject of clinical trial.

However, despite its promising activity, the compound readily isomerises to its inactive (E)-isomer and efforts have been directed towards the identification of configurationally stable analogues. Recently, independent researchers have identified compounds (B) (U.S. Pat. No. 5,886,025) and (C) (Madarde, M. et al Bioorg, Med. Chem Lett., 1999, 2303) as showing moderate tubulin binding and anti-mitotic activity. However, in contrast, the benzofuran (D) did not exhibit tubulin binding activity (Banwell, M. G., et al Aust J. Chem., 1999, 52, 767-774).

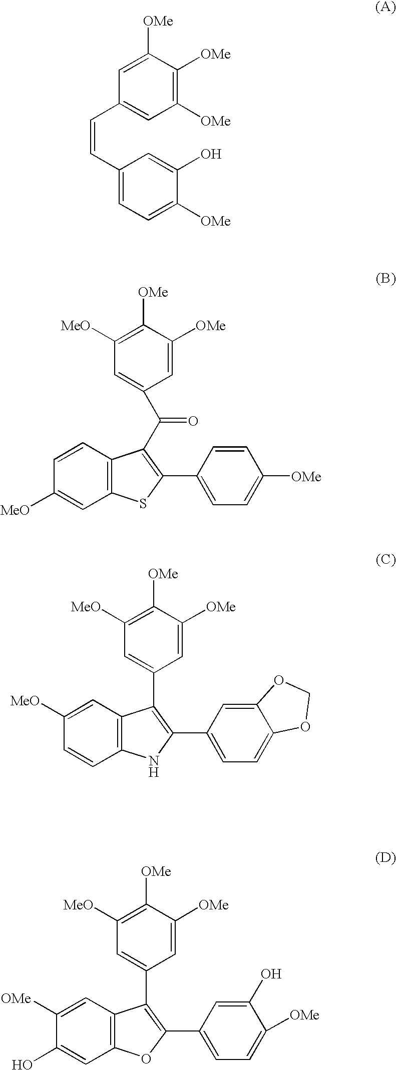

Accordingly, there exists a need for new synthetic methods which can allow for the generation of libraries of compounds which can be screened for desired bioactivities, such as tubulin binding and anti-mitotic activity, and for the discovery of new compounds which possess this activity.

It has now been found that a number of compounds prepared by new synthetic methods have been found to possess useful bioactivity. These compounds can be variously prepared from a small group of starting substrates to provide a combinatorial library of compounds which can be screened for bioactivity.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference to any prior art in this specification is not, and should not be taken as, an acknowledgment or any form of suggestion that that prior art forms part of the common general knowledge in Australia.

The present invention provides new methods for generating libraries of compounds including benzothiophenes, benzofurans, indoles, indanones and indenones, as well as non-benzo-fused analogues, for biological screening. The invention further provides new compounds which exhibit tubulin binding anti-mitotic activity, and processes for their preparation.

In a first aspect, there is provided a combinatorial library of 2 or more chemical compounds each compound comprising the reaction product derived from at least two substrates selected from (a), (b) and (c): 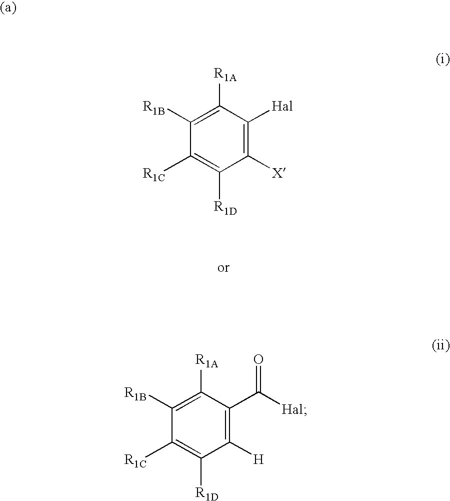
(b) 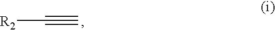
or a metallated form thereof; or 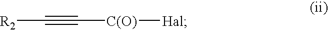
   or 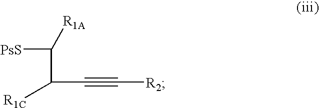
(c)
   (i) R₃-L, or a metallated form thereof wherein L is replaced by a metal; or
   (ii) R₃—C(O)-Hal;
      wherein
R_{1A}-R_{1D} are independently selected from hydrogen, hydroxy, optionally substituted alkyl, optionally substituted alkoxy, optionally substituted acyloxy, amino, optionally substituted alkylamino, optionally substituted dialkylamino or any 2 adjacent R_{1A}-R_{1D} together form
   —O—CH₂—O—;
Hal is I, Br or Cl;
X′ is OH, SPs (wherein Ps is a sulfur-protecting group capable of stabilising a positive charge), NP_{N} (wherein P_{N} is a nitrogen-protecting group), or NHR (wherein R is sulfonyl, trifluoroacyl, C₁₋₇acyl, C₁₋₆alkyl, or an aryl group);
R₂ and R₃ are optionally substituted aryl groups;
L is a leaving group.

In another aspect, the invention provides for combinational library of compounds for screening, as potential tubulin polymerisation inhibitors, said library comprising two or more compounds of formulae (E) to (O), said compounds being the reaction products of the following substrates:
(a)(i), (b)(i) and (c)(i) to produce compounds of formulae (E) and (F) 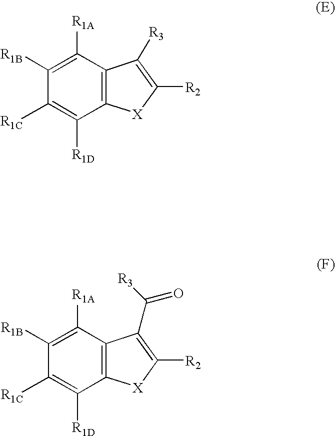
(a)(i), (b)(i) and (c)(ii) to produce compounds of formula (F);
(a)(i), (b)(ii) and (c)(i) to produce compounds of formulae (G), (H), (1), (J) or (K) 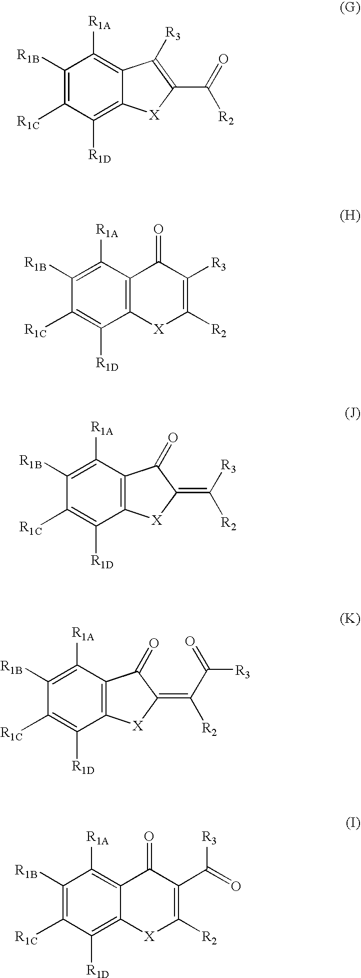
(c)(ii) to produce compounds of formulae (I) and (K)
(c)(i) to produce compounds of formula (L) 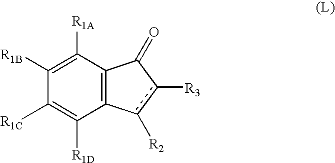
(a)(ii), (b)(i) and (c)(ii) to produce compounds of formula (M) 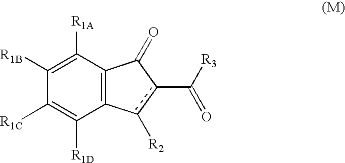
(b)(iii) and (c)(i) to produce compounds of formula (P) 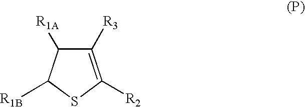
(b)(iii) and (c)(ii) to produce compounds of formula (O) 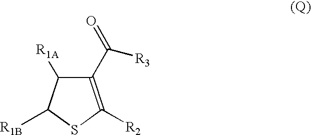
wherein R₂, R₃, R_{1A}-R_{1D}, (a)(i), (a)(ii), (b)(i)-(b)(iv) and (c)(i)-(iii) are as defined above, and X═O, S, or NR (wherein R is H, sulfonyl, C₁₋₆alkyl, C₁₋₇acyl, or an aryl group).

In a further aspect the present invention provides a combinational library of intermediates useful for the preparation of compounds of formulae (E)-(O), said intermediates being the reaction products of the following substrates;
(a)(i) and (b)(i) to produce intermediates of formula (E′) for use in preparing compounds of formulae (E) and (F) 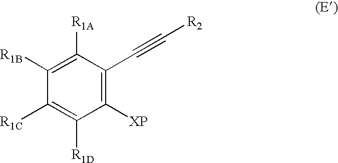
(a)(i) and (b)(ii) to produce intermediates of formulae (F′), (G′) and (H′) for use in preparing compounds of formulae (G), (H), (I), (J) or (K); 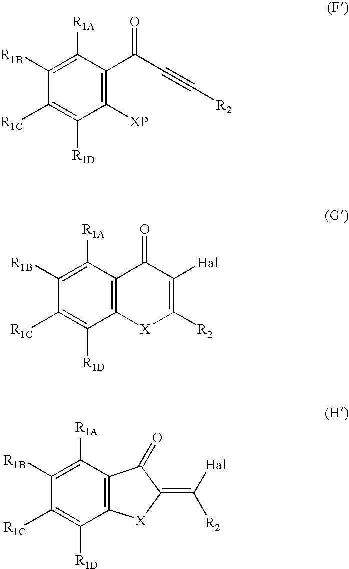
(a)(ii) and (b)(i) to produce intermediates of formulae (I′) and (J′) for use in preparing compounds of formula (L) or (M) 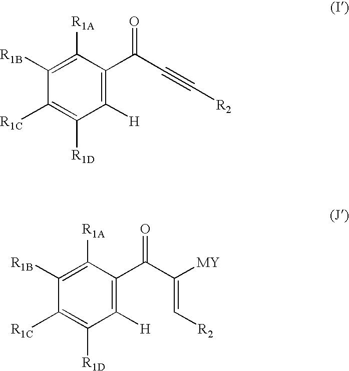
(b)(iii) with itself to produce intermediates of formulae (L′) 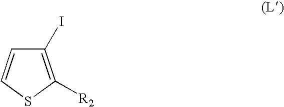
for use in preparing compounds of formulae (P) and (O);
wherein R_{1A}-R_{1D}, R₂, R₃, X (a)(i), (a)(ii), (b)(i)-(iv) and (c)(ii)-(iii) are as defined above, X is N, O or S, P is a protection group and MY is Sn(alkyl)₃ or B(OR)₂, wherein R is H or alky.

In another aspect, the invention provides a combinatorial library of at least two compounds of formula (I): 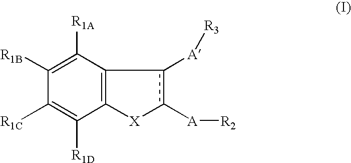
wherein
X is selected from O, S, NR, C═O(R is H, C₁₋₆alkyl or C₁₋₆acyl);
A and A′ are independently selected from CH₂, C═O, CH(OR′) (R′ is H, C₁₋₆alkyl, C₁₋₇acyl) or a single bond; provided that when one of A or A′ is CH₂, C═O or CH(OR), then the other is a single bond and that when X is S or NH, A is CH₂, C═O or CH(OR); 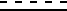
   is a double bond when X is O, S or NR; or
   is a single or double bond when X is C═O.
R_{1A}-R_{1D} are independently selected from hydrogen, hydroxy, optionally substituted alkoxy, optionally substituted alkyl, optionally substituted acyloxy, amino, optionally substituted alkylamino, optionally substituted dialkylamino, optionally substituted acylamino or any 2 adjacent R_{1A}-R_{1D} together form —O—CH₂—O—.
R₂ and R₃ are optionally substituted aryl groups.

In a further aspect the invention provides a compound of formula (I′″″) 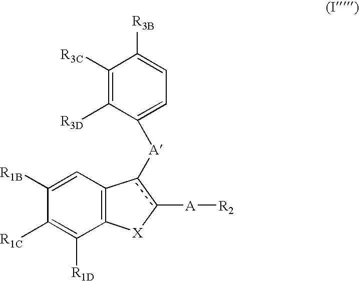
wherein X is O, S, NR (wherein R is hydrogen, sulfonyl, C₁alkyl, C₁₋₇acyl, or an aryl group) or C═O; and
R_{1B}-R_{1D} and R_{3B}-R_{3D} are independently selected from hydrogen, hydroxy, methoxy, and amino or any 2 adjacent R₁ and/or R₃ groups from R_{1B}-R_{1D} and R_{3B}-R_{3D} form a dioxolanyl group;
R₂ is an optionally substituted aryl group;
A and A′ are independently selected from the group consisting of a single bond, C═O, CH₂, and CH(OR′), (wherein R′ is hydrogen, C₁₋₆alkyl or C₁₋₇acyl); and
provided that the compound is not;
3-(3′,4′,5′-trimethoxybenzoyl)-2-(4′-methoxyphenyl)-6-methoxybenzo[b]thiophene;
3-(2′,6′-dimethoxybenzoyl-2-(4′-methoxyphenyl)-6-methoxybenzo[b]thiophene;
3-(3,5′-dimethoxybenzoyl)-2-(4′-methoxyphenyl)-6-methoxybenzo[b]thiophene;
3-(3′,4′-dimethoxybenzoyl)-2-(4′-methoxyphenyl)-6-methoxybenzo[b]thiophene;
3-(4′-methoxybenzoyl)-2-(4′-methoxyphenyl)-6-methoxybenzo[b]thiophene;
3-(4′-ethoxybenzoyl)-2-(4′-methoxyphenyl)-6-methoxybenzo[b]thiophene;
3-(3′,4′,5′-triethoxybenzoyl)-2-(4′-methoxyphenyl)-6-methoxybenzo[b]thiophene;
3-[3′-(3′,4′,5′-trimethoxyphenyl)propionyl]-2-(4′-methoxyphenyl)-6-methoxybenzo[b]thiophene;
3-(3′,4′,5′-triethoxybenzoyl)-2-(4′-ethoxyphenyl)-6-ethoxybenzo[b]thiophene;
3-(4′-ethoxy-3′,5′-dimethoxybenzoyl)-2-(4′-methoxyphenyl)-6-methoxybenzo[b]thiophene;
3-(4′-N,N-dimethylaminobenzoyl)-2-(4′-methoxyphenyl)-6-methoxybenzo[b]thiophene;
3-(3′,4′,5′-triflurobenzoyl)-2-(4′-methoxyphenyl)-6-methoxybenzo[b]thiophene;
3-(2′,3′,4′,5′,6′-pentafluorobenzoyl)-2-(4′-methoxyphenyl)-6-methoxybenzo[b]thiophene;
3-(3′,4′,5′-trimethoxybenzoyl)-2-(4′-methoxyphenyl)-benzo[b]thiophene;
3-(3′,4′,5′-trimethoxybenzoyl)-2-(4′-ethoxyphenyl)-6-ethoxybenzo[b]thiophene;
3-(4′-hydroxy-3′,5′-dimethoxybenzoyl)-2-(4′-methoxyphenyl)-6-methoxybenzo[b]thiophene;
2-(3′,4′,5′-trimethoxybenzoyl)-3-(4′-methoxyphenyl)-6-methoxybenzo[b]furan;
2-(4′-methoxyphenyl)-3-(3′,4′,5′-trimethoxybenzoyl)-6-methoxyindole;
2-(3′-t-butylsiloxy-4′-methoxyphenyl)-3-(3′,4′,5′-trimethoxybenzoyl)-6-methoxyindole; Disodium 2-(4′-methoxyphenyl-3′-O-phosphate)-3-(3″,4″,5″-trimethoxybenzoyl)-6-methoxyindole;
2-(4′-methoxyphenyl)-3-(3″,4″,5″-trimethoxybenzoyl)-4-methoxyindole; Disodium 2-(3′-phosphormaidate-4′-methoxyphenyl)-3-(3″, 4″,5″-trimethoxybenzoyl)-6-methoxyindole;
2-(3′-hydroxy-4′-methoxyphenyl)-3-(3″,4″,5″-trimethoxybenzoyl)-4-methoxyindole;
2-(3′-amino-4′-methoxyphenyl)-3-(3″,4″,5″-trimethoxybenzoyl)-4-methoxyindole;
Disodium 2-[(4′-methoxyphenyl)-3′-O-phosphate]-3-(3″,4″,5″-trimethoxybenzoyl)-4-methoxyindole;
2-(3′-diethylphosphoramidate-4′-methoxyphenyl)-3-(3″,4″,5″-trimethoxybenzoyl)-4-methoxyindole;
Disodium 2-(3′-phosphoramidate-4′-methoxyphenyl)-3-(31″,4″, 5″-trimethoxybenzoyl)-4-methoxyindole;
2-(1-napth-1-yl)-3-(3″,4″,5″-trimethoxyphenyl)-5-methoxyindole;
2-(3,4-methylenedioxyphenyl)-3-(3″,4″,5″-trimethoxyphenyl)-5-methoxyindole;
2-(furan-2-yl)-3-(3′,4′,5′-trimethoxyphenyl)-5-methoxyindole;
2-(furan-3-yl)-3-(3′,4′,5′-trimethoxyphenyl)-5-methoxyindole;
2-(5-methylfuran-2-yl)-3-(3′,4′,5′-trimethoxyphenyl)-5-methoxyindole.

In a further aspect, the invention provides a method of preparing a compound of formula (I′) 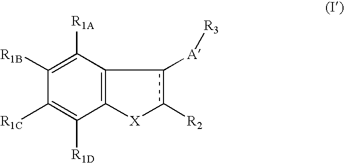
wherein
X is O, NH or NR, (wherein R is H, sulfonyl C₁₋₆alkyl or C₁₋₇acyl);
A′ is independently selected from a single bond, CH₂, C═O, and CH(OR′) (wherein R′ is H, C₁₋₆alkyl or C₁₋₇acyl);
R_{1A}-R_{1D} are independently selected from hydrogen, hydroxy, optionally substituted alkoxy, optionally substituted alkyl, optionally substituted acyloxy, amino, optionally substituted alkylamino, optionally substituted dialkylamino, optionally substituted acylamino, or any 2 adjacent R_{1A}-R_{1D} together form —O—CH₂—O—;
R₂ and R₃ are optionally substituted aryl groups;
said method comprising the steps of:
a) coupling a compound of formula (1) with an alkyne of formula (2) in the presence of a nickel or palladium coupling agent 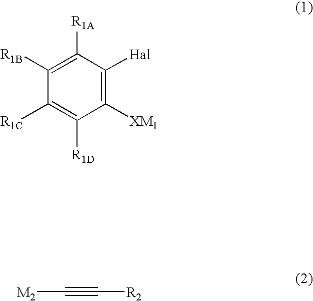
   wherein
R_{1A}-R_{1D}, R₂ and X are as above; Hal is I, Br or Cl;
M₁ is a metal or a metal species therof, said metal selected from the group consisting of Li, Na, K, Mg, Cs and Ba;
M₂ is a metal, or a metal species thereof, said metal selected from the group consisting of Mg, Zn, Cu, B, Si, Mn, Sn, Ge and Al;
X is O or NR (wherein R is sulfonyl, C₁₋₆alkyl, or C₁₋₇acyl);
b) reacting in situ the resulting coupled product with R₃-L, wherein R₃ is an optionally substituted aryl group and wherein L is a leaving group, optionally in the presence of carbon monoxide; and
c) optionally reducing the resulting product, when A′ is C═O, to afford compounds in which A═CH₂ or CH(OR′).

In yet another aspect, the invention provides a method for preparing a compound of formula (I″): 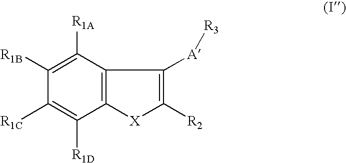
wherein
X is S;
A′ is selected from a single bond, CH₂, C═O, and CH(OR′) (wherein R′ is H, C₁₋₆alkyl or C₁₋₇acyl);
R_{1A}-R_{1D} are independently selected from hydrogen, hydroxy, optionally substituted alkoxy, optionally substituted alkyl, optionally substituted optionally substituted acyloxy, amino, optionally substituted alkylamino, optionally substituted dialkylamino, optionally substituted acylamino, or any two adjacent R_{1A}-R_{1D} together form —O—CH₂—O—;
R₂ and R₃ are optionally substituted aryl groups;
said method comprising the steps of:
a) coupling a compound of formula (3) with a compound of formula (4) in the presence of a nickel or palladium coupling agent 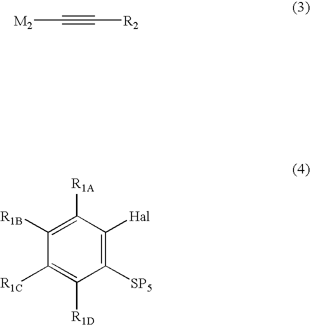
   wherein
R_{1A}-R_{1D}, Hal, M₂ and R₂ are as above, and Pₛ is a sulfur protecting group capable of stabilizing a positive charge;
b) cyclising the resulting coupled product in the presence of a Hal⁺ producing reagent to give (5) 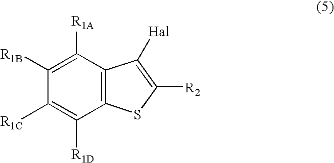
   wherein
Hal is Cl, Br or I;
c) coupling (5) with either the moiety R₃—C(O)— or R₃— wherein R₃ is an optionally substituted aryl group; and
d) optionally reducing the coupled product when A′ is C═O, to afford compounds in which A′═CH₂ or CH(OR′).

In another aspect, the invention provides a method for preparing a compound of formula (I′″) 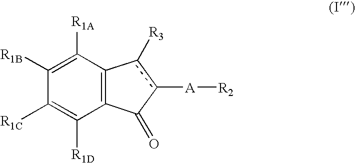
wherein
A is selected from a single bond, CH₂, C═O and CH(OR′) (wherein R′ is H, C₁-alkyl or C₁₋₇acyl);
R_{1A}-R_{1D} are independently selected from hydrogen, hydroxy, optionally substituted alkoxy, optionally substituted alkyl, optionally substituted acyloxy, amino, optionally substituted alkylamino, optionally substituted dialkylamino or any 2 adjacent R_{1A}-R_{1D} together form —O—CH₂—O; 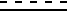
   is an optional double bond;
R₂ and R₃ are optionally substituted aryl groups;
said method comprising the steps of:
(a) reacting compound (6) with compound (7) or reacting compound 6(a) with compound 7(a). 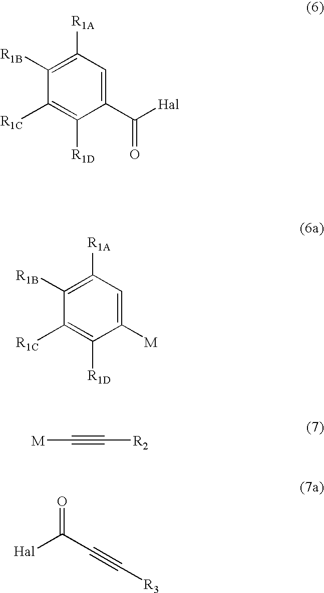
   to form a compound (9) 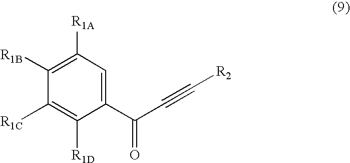
   wherein
M is Li, Na, K or MgHal (Hal is Br, Cl or I);
b) treating compound (9) with a metal hydride in the presence of a palladium coupling agent;
c) coupling the resulting product with R₃-Hal or R₃—C(O)-Hal (wherein Hal is Cl, Br or I) to provide either compound (10) or (11); and 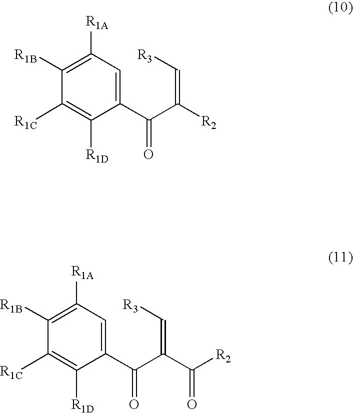
(d) cyclising (10) or (11) under acidic conditions to form an indanone and optionally treating the cyclised product with an oxidising agent to form an indenenone.

In yet a further aspect, the invention provides a method for preparing a compound of Formula (I″″) 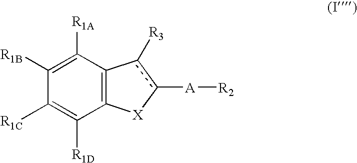
wherein;
X is O, S or NR (wherein R═H, C₁₋₆alkyl or C(O)C₁₋₆alkyl);
R_{1A}-R_{1D} are as previously defined;
A is C═O, CH₂ or CH(OR′) (wherein R′ is H, C₁₋₆alkyl or C₁₋₇acyl);
R₂ and R₃ are optionally substituted aryl groups;
comprising the steps of
a) coupling a compound (12) with compound (13) 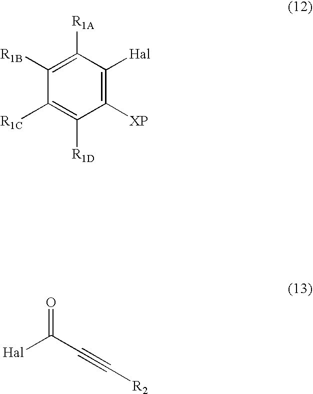
wherein Hal is Cl, Br, or I;
to form a compound of formula (14); 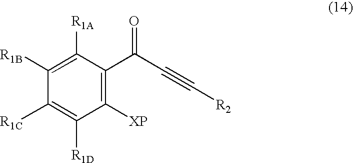
b) when X is S, protecting the thiol with a sulfur-protecting group
c) reacting (14) with a compound
   M₁—R₃
   wherein
M₁ is Li, Na, K, Mg, Cs or Ba, and R₃ is an optionally substituted aryl group; to form 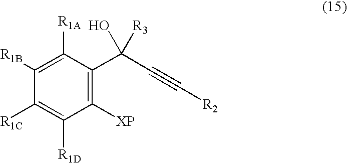
   wherein
when X is O or NH, then P is H and when X is S, P is a sulfur protecting group and when XP is NR, R is a hydrogen, sulfonyl, C₁₋₆alkyl, C₁₋₇aryl or an aryl group;
d) treating (15) with a Hal⁺ producing reagent, to afford cyclisation;
e) and optionally reducing the cyclised product when A′═CO, to afford libraries of compounds in which A′═CH₂ or CH(OR′).

Still another aspect of the invention provides a compound of Formula II 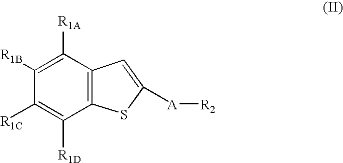
wherein
R_{1A}-R_{1D} are independently hydrogen, hydroxy, optionally substituted alkoxy, optionally substituted alkyl, optionally substituted optionally substituted acyloxy, amino, optionally substituted alkylamino, optionally substituted dialkylamino or 2 adjacent R_{1A}-R_{1D} are O—CH₂—O;
R₂ and R₃ are optionally substituted aryl groups;
A is C═O, CH₂ or CH(OR′) (R′═H, C₁₋₆alkyl or C₁₋₇acyl).

Still yet a further aspect of the invention relates to compounds of Formula (III) 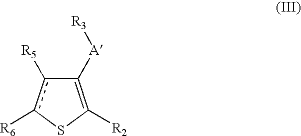
wherein
R₂ and R₃ are optionally substituted aryl groups;
A′ is CO, CH₂, CH(OR′) (wherein R′═H, C₁₋₆alkyl or C₁₋₇acyl) or a single bond;
R₅ and R₆ can independently be hydrogen, optionally substituted alkyl, optionally substituted aryl or optionally substituted alkenyl; 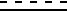
   is an optional double bond.

Other aspects of the invention relate to combinatorial libraries of compounds comprising at least two compounds of Formula (I′), (I″), (I′″) (I″″), (II) or (III).

Still other aspects of the invention relate to the use of the compounds of the present invention in the manufacture of medicaments, and methods thereof, in the treatment of conditions requiring tublin polymerization inhibitors.

### BRIEF DESCRIPTION OF THE DRAWINGS

The drawings which follow form part of the present specification and are included to further demonstrate certain aspects of the present invention. The invention may be better understood by reference to one or more of these drawings in combination with the detailed description of specific embodiments presented herein.

FIG. 1—schematically depicts a generalised “one pot” synthesis of benzofused furans and indoles.

FIG. 2—schematically depicts a generalised “one pot” synthesis of benzofused furans and indoles.

FIG. 3—schematically depicts a generalised synthesis of benzofused thiophenes.

FIG. 4—schematically depicts a generalised synthesis of benzofused thiophenes, including compound (B) from U.S. Pat. No. 5,886,025.

FIG. 5—schematically depicts a generalised synthesis of benzofused thiophenes.

FIG. 6—schematically depicts a generalised synthesis of benzofused thiophenes.

FIG. 7—schematically depicts a generalised “one pot” synthesis of aryl substituted α, β-alkenyl carbonyl compounds, for the preparation of indanones and indenones.

FIG. 8—schematically depicts a generalised synthesis of benzofused furans, indoles and thiophenes.

FIG. 9—schematically depicts a generalised synthesis of benzofused thiophenes.

FIG. 10—schematically depicts a generalised synthesis of thiophenes.

FIG. 11—depicts the structures of some of the preferred compounds of the present invention which posses TPI activity.

### DETAILED DESCRIPTION OF THE INVENTION

As used herein, the term “alkyl”, used either alone or in compound words such as “alkylamino” and “dialkylamino” etc, denotes straight chain, branched or cyclic alkyl, preferably C₁₋₂₀ alkyl, eg C₁₋₁₀ or C₁₋₆alkyl, Examples of straight chain and branched alkyl include methyl, ethyl, propyl, isopropyl, butyl, isobutyl, sec-butyl, tert-butyl, amyl, isoamyl, sec-amyl, 1,2-dimethylpropyl, 1,1-dimethyl-propyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2,-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethyl-pentyl, 1,2,3-trimethylbutyl, 1,1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-, 5-, 6- or 7-methyl-octyl, 1-, 2-, 3-, 4- or 5-ethylheptyl, 1-, 2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propylocytl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like. Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl and the like. Where an alkyl group is referred to generally as “propyl”, butyl” etc, it will be understood that this can refer to any of straight, branched and cyclic isomers where appropriate. “Alkoxy” refers to an alkyl when conveniently bonded to an oxygen atom. An alkyl group may be optionally substituted by one or more optional substituents as herein defined. Accordingly, “alkyl” as used herein is taken to refer to optionally substituted alkyl. Optional substituents for an alkyl group include hydroxy, halo, alkoxy, phenyl, benzyl, phenoxy, benzyloxy, carbonyl, amino, acyl, acyloxy, alkylamino, dialkylamino, acylamino. Particularly preferred optional substituents include those wherein the alkyl moiety of the substituent is C₁₋₆alkyl. An alkyl group may also contain one or more degrees of unsaturation and therefore, alkyl may also include groups as described above containing one or more double or triple bonds.

Optionally substituted alkoxy, alkylamino, dialkylamino refers to the optional substitution of the “alkyl” moiety. Similarly, optionally substituted, acyl, acylamino and acyloxy refer to the optional substitution of the alkyl or aryl moieties of the acyl group.

The term “alkenyl” as used herein denotes groups formed from straight chain, branched or cyclic hydrocarbon residues containing at least one carbon to carbon double bond including ethylenically mono-, di- or poly-unsaturated alkyl or cycloalkyl groups as previously defined, preferably C₁₋₂₀ alkenyl (eg C₁₋₁₀ or C₁₋₆). Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1-4,pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl. An alkenyl group may be optionally substituted by one or more optional substitutents as herein defined. Accordingly, “alkenyl” as used herein is taken to refer to optionally substituted alkenyl.

Unless indicated otherwise, the term “halogen”, “halo” “halide” etc. denotes fluorine, chlorine, bromine or iodine (fluoro, chloro, bromo or iodo) (fluoride, chloride, bromide iodide).

The term “aryl” includes single, polynuclear, conjugated and fused residues of aromatic hydrocarbon ring systems. Examples of hydrocarbon based “aryl” include phenyl, biphenyl, terphenyl, quaterphenyl, naphthyl, tetrahydronaphthyl, anthracenyl, dihydroanthracenyl, benzanthracenyl, dibenzanthracenyl, phenanthrenyl, fluorenyl, pyrenyl, idenyl, azulenyl, chrysenyl. The term “aryl” also includes cyclic (single, polynuclear, fused or conjugated) hydrocarbon residues where one or more carbon atoms are replaced by a heteroatom and form an aromatic residue where two or more carbon atoms are replaced, this may be by the same heteroatom or different heteroatoms. Suitable heteroatoms include O, N, S and Se. Such aryl residues can be referred to as “heteroaryl”. Suitable heteroaryl include furanyl, thieuyl, pyrrolyl, indolyl, pyridyl, pyridazinyl, pyrazoly, pyrazinyl, thiazolyl, pyrimidinyl, quinolinyl, isoquinolinyl, benzofuranyl and benzothienyl. An aryl group may be substituted by one or more substituents and accordingly “aryl” is taken herein to refer to optionally substituted aryl. Preferred optional substituents include are independently selected from hydrogen, hydroxy, alkoxy, alkyl, acyloxy, amino, alkylamino, dialkylamino or any 2 adjacent positions are substituted to together form —O—CH₂—O. An aryl group may also be optionally fused to a cyclic or polycyclic group (saturated or unsaturated), which itself may be further optionally substituted as described for “alkyl” above.

The term “acyl” either alone or in compound words such as “acyloxy”, or “acylamino” etc, denotes a group containing the moiety C═O (and not being a carboxylic acid, ester or amide or thioester) Preferred acyl includes C(O)—R, wherein R is hydrogen or an alkyl, or aryl preferably a C₁₋₂₀ residue. C₁₋₇acyl refers to an acyl group that counts the carbonyl group as one carbon atom. Examples of acyl include formyl; straight chain or branched alkanoyl such as, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g. phenylacetyl, phenylpropanoyl, phenylbutanoyl, phenylisobutylyl, phenylpentanoyl and phenylhexanoyl) and naphthylalkanoyl (e.g. naphthylacetyl, naphthylpropanoyl and naphthylbutanoyl]. The term “acyloxy” refers to an acyl group covalently bonded to an oxygen atom.

As used herein, the term “protecting group”, refers to an introduced functionality which temporarily renders a particular functional group inactive under certain desired conditions. Suitable protecting groups are known to those skilled in the art, for example as described in Protective Groups in Organic Synthesis (T. W. Greene and P. G. M. Wutz, Wiley Interscience, New York, 3^{rd} edition).

As used herein, the term “leaving group” refers to a chemical group which is displaced by a nucleophile. Suitable leaving groups include those with the ability to stabilise the negative charge which it carries such as the halogens (e.g. I, Br, Cl), triflate (e.g. trifluoromethane sulfonyl), acetate and sulfonates (eg. tosylate, mesylate, nosylate etc). Some preferred leaving groups are I, Br, Cl and trifluoromethane sulfonyl.

In preferred embodiments of the invention, R_{1A}-R_{1D} include hydrogen, hydroxy, optionally substituted C₁₋₆alkoxy, optionally substituted C₁₋₆alkyl, optionally substituted C(O)—C₁₋₆alkyl, amino, optionally substituted optionally substituted C₁₋₆alkylamino, optionally substituted diC₁₋₆alkylamino or 2 adjacent R_{1A}-R_{1D} form a dioxolanyl group (—O—CH₂—O). Particularly preferred R_{1A}-R_{1D} include: hydrogen, hydroxy, methoxy, ethoxy, propoxy, butoxy, methyl, ethyl, propyl, butyl, acetyl, acetyloxy, amino, methylamino, ethylamino, propylamino butylamino, dimethylamino, diethylamino, dipropylamino, dibutylamino or 2 adjacent R_{1A}-R_{1D} form a dioxolanyl group.

In yet other preferred embodiments of the invention, R₂ and R₃ can be independently an optionally substituted phenyl group of formula (i): 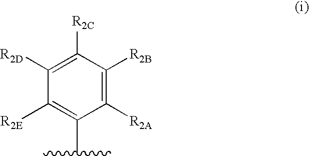
wherein
R_{2A}-R_{2E} are independently selected from hydrogen, hydroxy, optionally substituted alkoxy, optionally substituted alkyl, optionally substituted acyloxy, amino, optionally substituted alkylamino, optionally substituted dialkylamino, optionally substituted acylamino or where any two adjacent R_{2A}-R_{2D} together form —O—CH₂—O—. Preferred R_{2A}-R_{2E} are as for R_{1A}-R_{1D} described above. Preferred forms of formula (i) are where R_{2A}-R_{2E} are independently hydrogen, hydroxy, methoxy, amino or any two adjacent R_{2A}-R_{2D} together form —O—CH₂—O—.

Where A or A′ is CH₂ or CH(OR′) these can be formed by the reduction of, C═O using a suitable reducing agent such as LiAlH₄ or NaBH₄. Alternatively, a CH(OH) group can be oxidized up to the carbonyl.

In the process for the preparation of compounds of Formula (I′), compounds (1) and (2) are derived from their respective phenol or protected amine and terminal alkyne respectively. The starting phenol or aniline and terminal alkyne can be coupled together under conditions which allow for the heteroannulation to spontaneously occur so as to form the target benzo[b]furan or indole in a “one-pot” synthetic strategy. Thus, the metal based compound required to form (1) must be such that the phenol or protected amine is deprotonated to form the group —OM₁ or NHM₁.

Suitable M₁ are based on Li, Na, K, Mg, Cs and Ba as well as species formed thereform, for example from Grignard reagents C₁₋₄alkyl MgHal (Hal=I, Cl or Br). Suitable metal species include MgCl, MgBr or MgI. Formation of (1) can be effected by treating the corresponding phenol or protected amine with, for example, Li₂CO₃, Na₂CO₃, K₂CO₃, MgCO₃, Cs₂CO₃, BaCO₃, MeMgCl, EtMgCl, MeMgBr, EtMgBr, MeMgI and EtMgI.

M₂ can be a hydrogen atom or metal species used in any palladium or nickel cross-coupling protocols known in the art, (for example, Stille, Suzuki or Negishi cross-coupling reactions using stannanes (eg aryl or alkylstannanes, boronic acids/esters or zinc based compounds eg. ZnCl) for example based on Mg, Zn, Cu, B, Si, Mn, Sn, Ge or Al. Particularly suitable M₂ include ZnCl, (alkyl)₃Sn, (aryl)₃Sn, B(OR)₂ (R is, eg, H or alkyl), MgBr, MgCl and MgI.

In a particularly preferred form of this aspect of the invention both M₁ and M₂ are derived from a Grignard reagent such as an alkyl magnesium halide eg. C₁₋₄alkylMgBr, (Cl) or (I). Suitable M₁ and M₂ thus include MgCl, MgBr and MgI.

Where X is NR in formula (I′), the nitrogen atom of the starting aniline is suitably protected by a nitrogen protecting group. Suitable nitrogen protecting groups are known to those skilled in the art of organic synthesis and include acyl groups (eg acetyl, trifluoroacetyl), phenyl, benzyl and benzoyl. Other suitable nitrogen protecting groups may be found in Protective Groups in Organic Synthesis, T. W. Greene and P. Wutz, John Wiley & Son, 3^{rd} Edition.

The coupling agent used in step (a) of the process for the preparation is preferably a nickel or palladium based coupling agent. Suitable coupling agents are known in the art and include Pd(PPh₃)₂Cl₂, Pd(PPh₃)₄, Pd(dibenzylideneacetone)₃ and PdCl₂(CH₃CN)₂.

The leaving group of R₃-L can be any suitable leaving group known to the skilled person. In a preferred embodiment, where R₃ is an optionally substituted aryl, L can be a halogen such as iodine, chlorine or bromine, a triflate, or a sulfonate (eg tosylate, mesylate, brosylate nosylate etc).

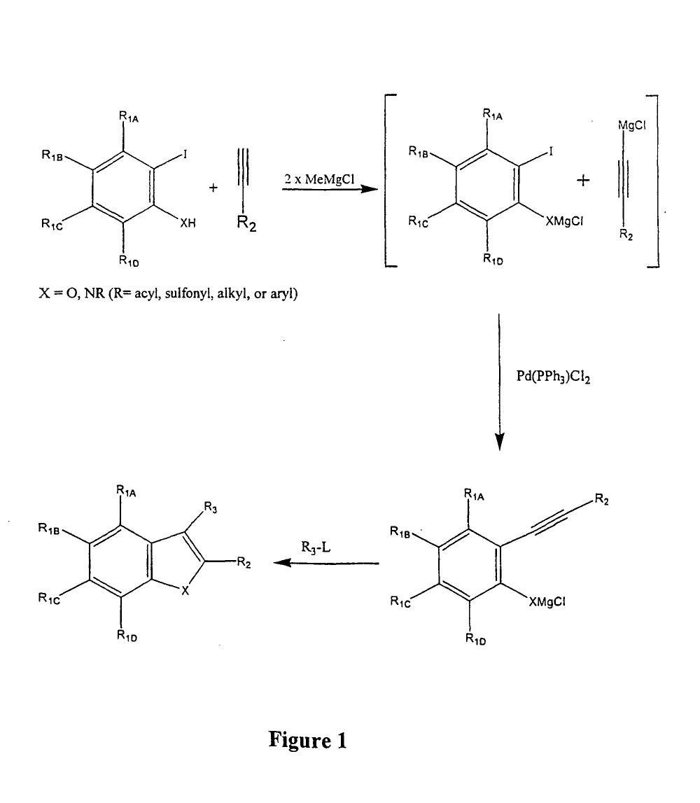

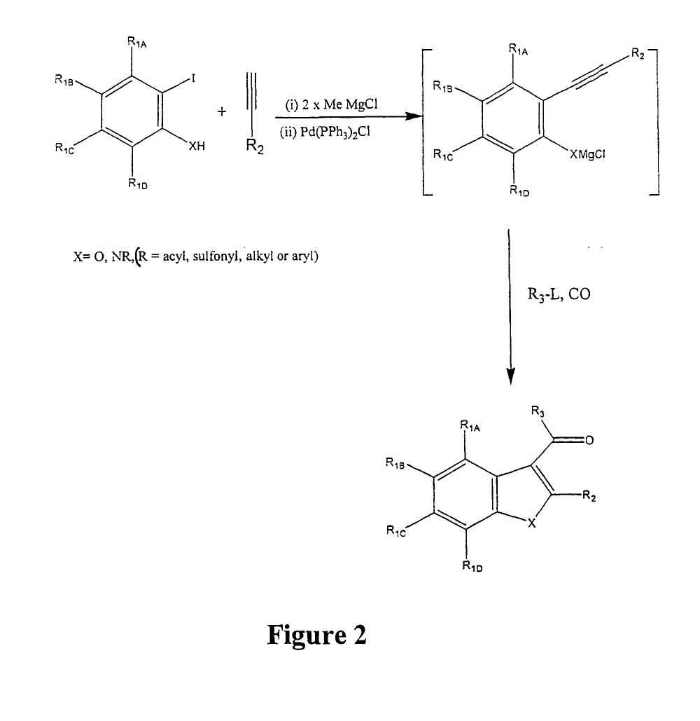

Two preferred embodiments of the process for preparing compounds of formula (I′) are schematically depicted in FIGS. 1 and 2.

The preparation of benzo[b]thiophenes of formula (I″) is effected using a variation of the methods described for the benzo[b]furans and indoles of formula (I′) above. In particular, the sulfur atom, X, must be protected by a suitable protecting group to circumvent competitive coupling of a thiolate to the aryl halide to afford xanthones. Suitable sulfur protecting groups are those which are capable of stabilizing a positive charge. Examples include benzyl, allyl, acetyls and thioacetals.

As used herein a Hal⁺ producing agent is an agent which can effectively act as a Hal⁺ source. Examples of Hal⁺ producing agents include I₂, Br₂, Cl₂, IBr, Icl, chloroacetamide, iodoacetamide, N-chlorosuccinamide, N-bromosuccinamide and N-iodosuccinamide.

Suitable M₂ for compound (2) and coupling agents for the preparation of compounds of Formula (I″) may include those described above.

The coupling of (4) with the moiety R₃—C(O)— or R₃— to produce (I″) can be carried out via palladium-mediated coupling and/or metallation techniques as known in the art. For example, lithiation of (4) (eg using nBuLi) allows for coupling with R₃—C(O)-Hal (Hal is I, Br or Cl, preferably Cl). In another embodiment, Negishi coupling of (4) with R₃—ZnCl (derived from reaction of R₃—Li with ZnCl) gives access to compounds of formula (I″) where A is a single bond. In another embodiment, palladium-mediated couplings such as Suzuki or Stille couplings can be used to access compounds of formula (I″).

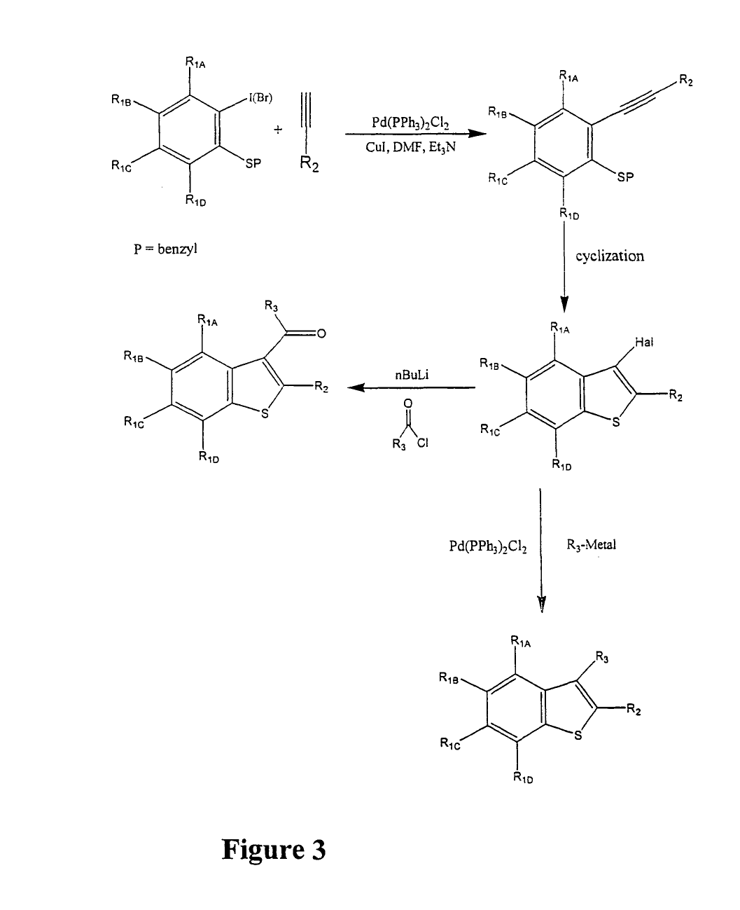

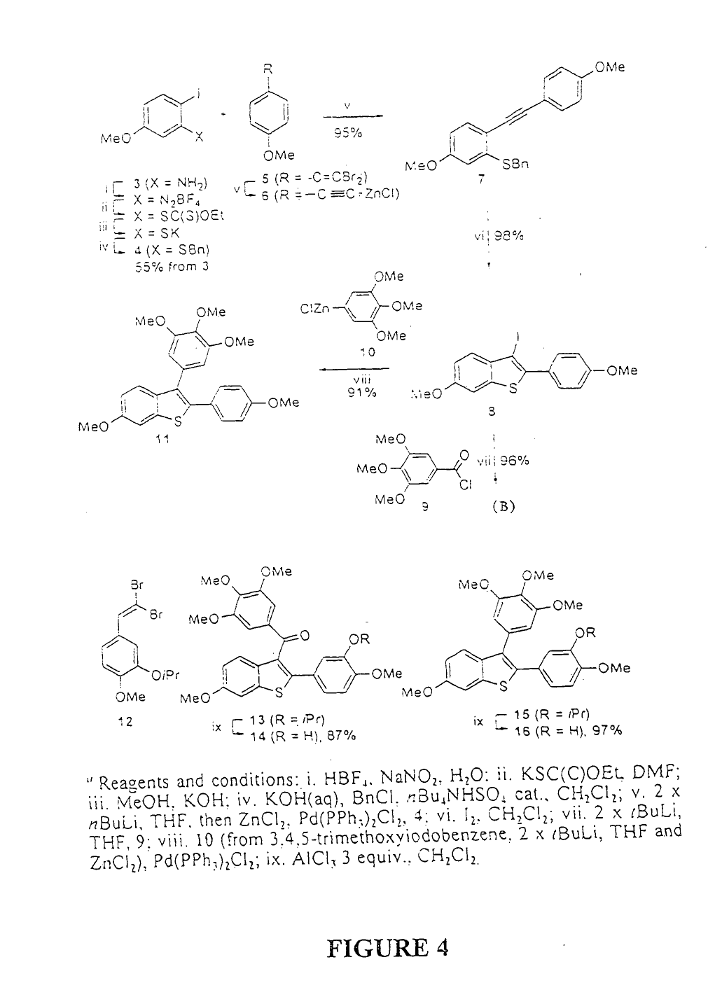

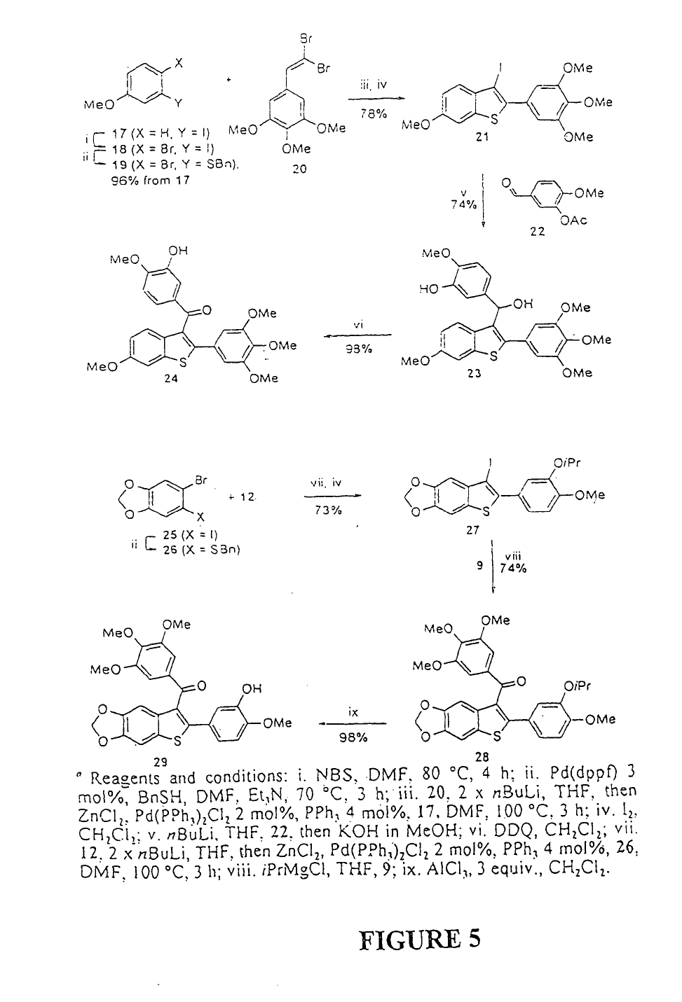

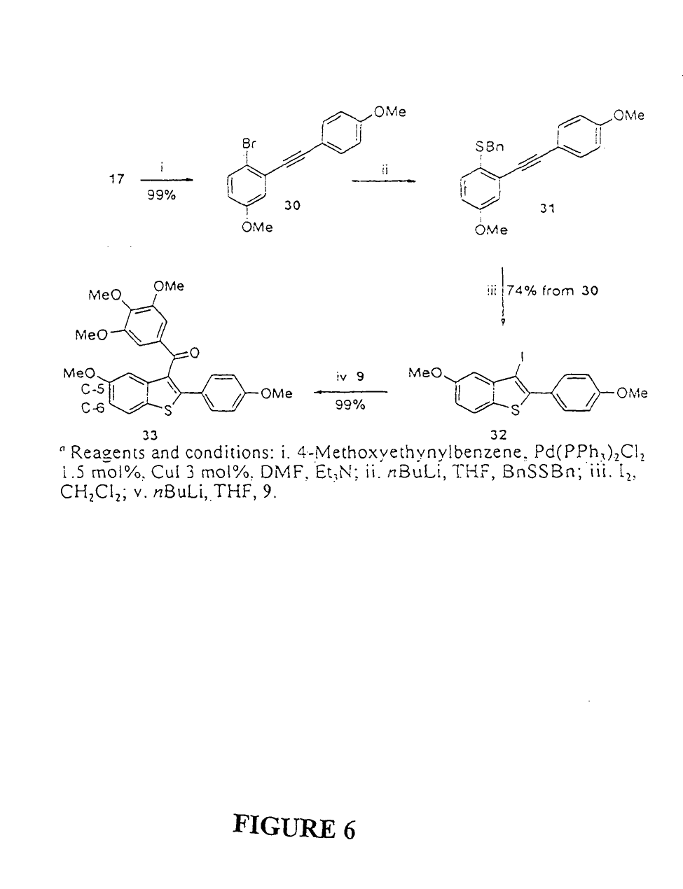

Some preferred embodiments for the preparation of Compounds of Formula (I″) are shown in FIGS. 3, 4, 5 and 6.

In the preparation of compounds of Formula (I′″), step (b) involves the use of a metal hydride. Suitable metal hydrides are those which react with the triple bond to form an intermediate metallated vinyl group without reducing the adjacent carbonyl group and may include trialkylstannanes (eg trimethyl- or tributyl tin hydride), aryl stannanes (eg triphenyl tin hydride), copper hydride, diisobutylaluminiumhydride, or borohydrides (eg catechol borane).

The cyclization step (d) employs acidic conditions, which can be either a H⁺ source or a Lewis acid. Suitable acids include HCl, H₂SO₄, BF₃, AlCl₃, methanesulphonic acid etc. A preferred oxidizing agent is DDQ.

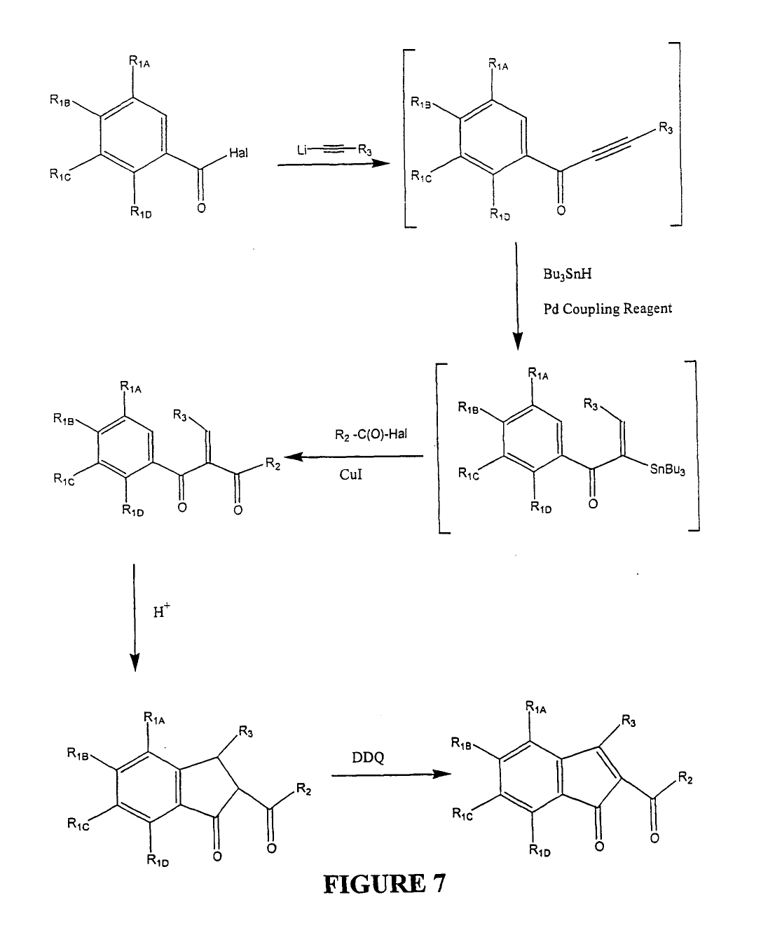

A schematic representation of one method for the preparation of compounds of Formula (I′″) is depicted in FIG. 7.

In the preparation of compounds of Formula (I″″), the coupling of (12) and (13) can be carried out using suitable metallation techniques known in the art. Thus the coupling can be carried out in the presence of n-BuLi sec-BuLi, t-BuLi or alkylMghalides such as iPrMgHalide.

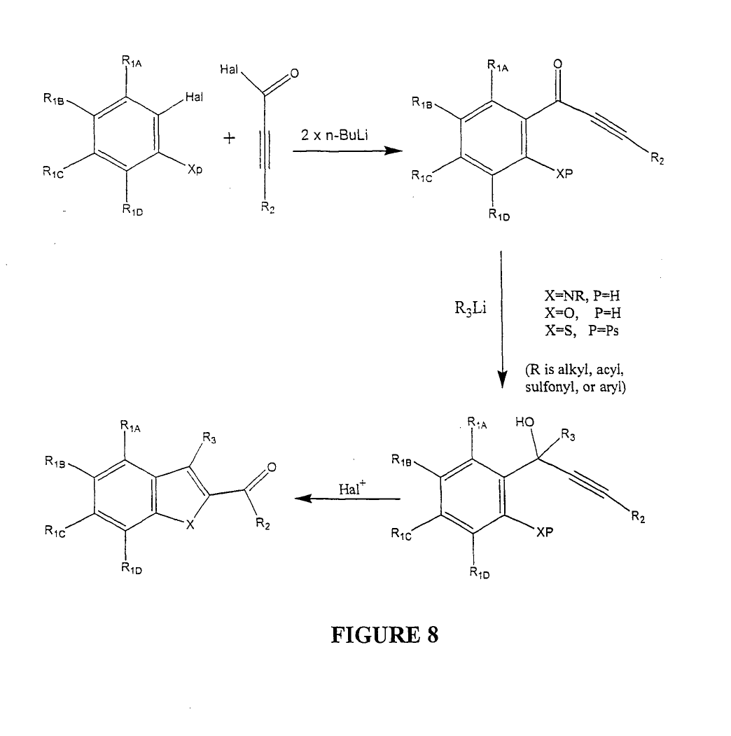

A schematic representation for a method of preparing compounds of Formula (I″″) is shown in FIG. 8.

Compounds of Formula (II) can be prepared by coupling of a suitably protected thiol aldehyde under Negishi conditions, lithiation and coupling with the appropriate ZnCl acetylide. Cyclisation using a Hal+ producing agent, eg iodocyclization using I₂, affords access to Formula (II).

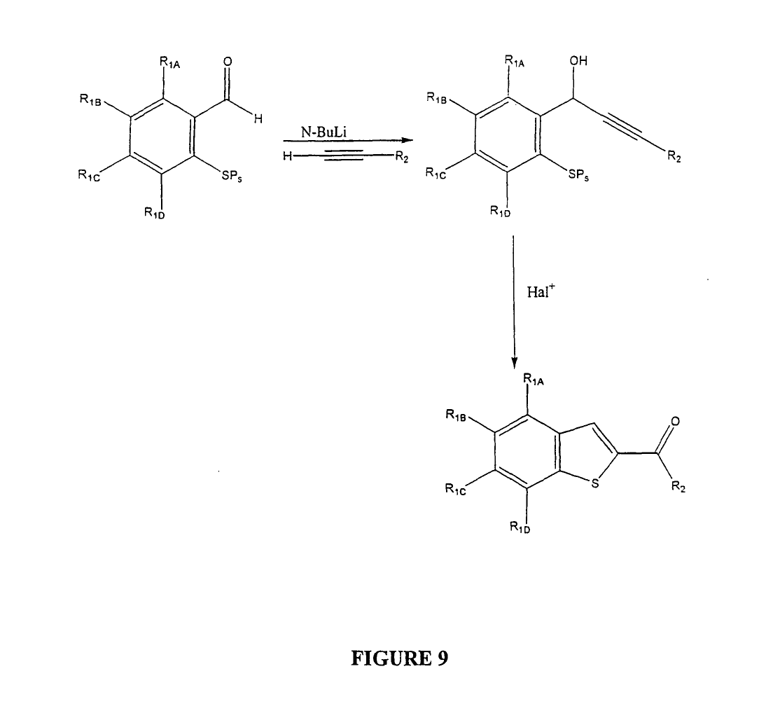

A schematic representation for a method of preparing compounds of Formula (II) is shown in FIG. 9.

Compounds of Formula (III) can be prepared by palladium-mediated coupling of an appropriately protected butynyl sulfide with R₂-L, for example under Sonagashira conditions using CuI. Other palladium-mediated coupling procedures such as Stille, Suzuki, and Negishi conditions can also be used. Cyclization can be effected using a Hal+producing agent as described herein. Coupling of the resulting Hal-dihydrothiophene with an R₃ moiety, for example, using ZnCl—R₃, provides products where A′ is a single bond. Alternatively, coupling with R₃—C(O)-Hal in the presence of nBuLi as described herein provides access to compounds of Formula (III) wherein A′ is C═O, CH₂ or CH(OR′).

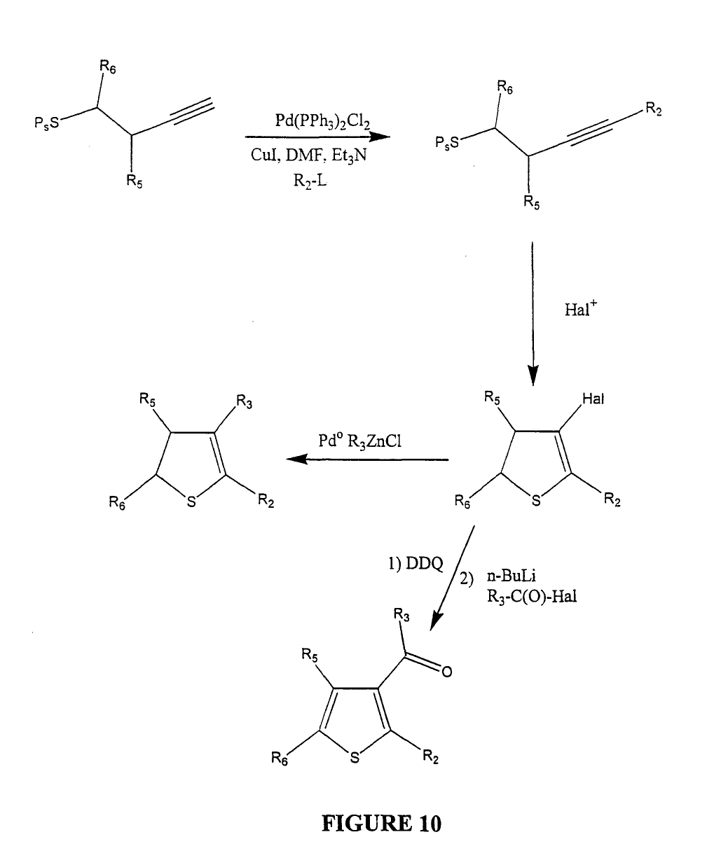

A schematic representation for a method of preparing compounds of Formula III is depicted in FIG. 10.

An important aspect of the present invention relates to compounds which may possess tubulin binding activity. Compounds which possess tubulin binding activity may act as anti-mitotic agents and may be effective in targeting tumour vasculature.

Thus, the invention also provides compounds of Formula (I′″″) 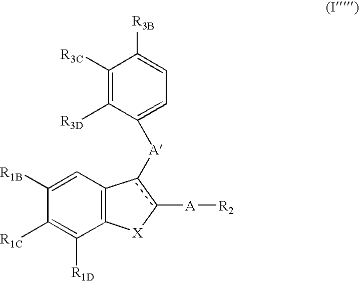
wherein X is O, S, NR (wherein R is hydrogen, sulfonyl, C₁-alkyl, C₁₋₇acyl or an aryl group) or C═O; and 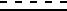
   is an optional double bond;
R_{1B}-R_{1D} and R_{3B}-R_{3D} are independently selected from hydrogen, hydroxy, methoxy, and amino or any 2 adjacent R_{1B}-R_{1D} and R_{3B}-R_{3D} from a dioxolanyl group;
R₂ is an optionally substituted aryl, preferably an optionally substituted phenyl group of formula (i) as herein described.
A and A′ are independently selected from the group consisting of a single bond, C═O, CH₂, and CH(OR′), (R′ is hydrogen, C₁₋₆alkyl or C₁₋₇acyl) provided that when one of A or A′ is C═O, CH₂ or CH(OR′), then the other is a single bond; and provided that when X is S or NH, A is C═O, CH₂ or CH(OR′);
with the proviso that when R_{1B} is methoxy, R_{1C} and R_{1D} are both H, A and A′ are both a single bond, R_{3B}-R_{3D} are each methoxy and R₂ is 3,4-dioxolanylphenyl, then X is not NH; and that when R_{1B} is methoxy, R_{1C} is hydroxy, A and A′ are both a single bond, R_{3B}-R_{3D} are each methoxy and R₂ is 3-hydroxy, 4-methoxyphenyl, then X is not O.

In a preferred form of (I′″″), X is O, C═O or NR, more preferably O.

In another preferred form of (I′″″) 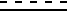
is a double bond.

In another preferred embodiment of (I′″″), when R₂ is a phenyl group of formula (i), at least one of R_{1B}-R_{1D} and R_{3B}-R_{3D} is a hydroxy or amino group which can be derivatized to form a salt or prodrug, such as an ester or amide, preferably a disodium phosphate ester.

In another preferred embodiment of (I′″″), when R₂ is a phenyl group of formula (i), the phenyl group bears vicinal hydroxy and methoxy groups.

In another preferred embodiment of (I′″″) at least one of A or A′ is C═O, CH₂, CH(OR′). More preferably, when X is O, A′ is C═O, CH₂ or CH(OR′).

Yet other preferred compounds which may possess tubulin binding activity have the formula (IV): 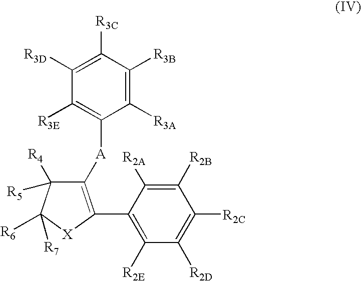
X is O, S or NR″ (wherein R″ is aryl, aroyl, acyl, benzyl, alkyl or sulfonyl)
A is a single bond C═O, CH(OR′) (R′ is hydrogen, C₁₋₆alkyl, C₁₋₇acyl), CH₂, O, S or NR (wherein R is hydrogen, C₁₋₆ hydrogen, C₁₋₆alkyl or C₁₋₇acyl).
R_{2A}-R_{2E} and R_{3A}-R_{3E} are as hereinbefore described.
R₄-R₇ are independently selected from hydrogen, hydroxy, alkoxy, alkyl, amino, alkylamino, dialkylamino, acyl, acylamino and aryl, or R₄ and R₇ together form a bond.

The compounds of Formula (IV) can be prepared using iodocyclisation procedures known for forming compounds where X═O or N (Bev, S. P. et al. Chem. Commun. 1996, 1007 and Knight, D. W. et al. Chem. Commun. 1998, 2207) or for where X═S, analogous methodology as described herein.

Couplings of the aryl groups R₂ and R₃ can be performed as described herein and for where A is O, S or NH, see Bavanno, D., et al, Curr. Org. Chem. 1997, 3, 287 and references cited therein.

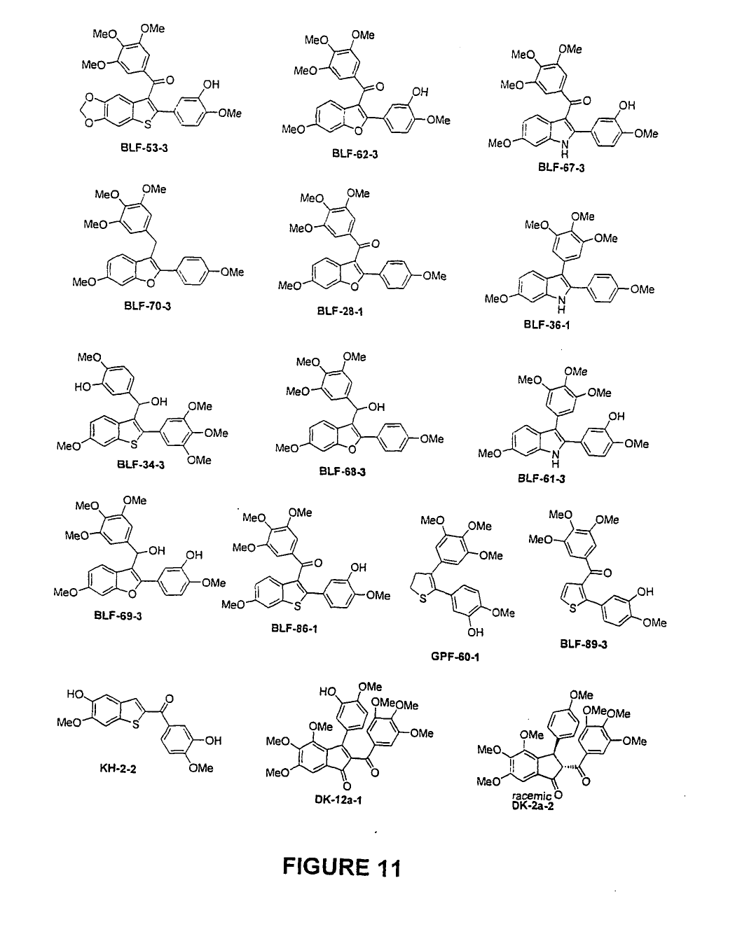

Some examples of compounds of the invention which possess tubulin-binding activity are depicted in FIG. 11.

Certain compounds of the invention having tubulin binding activity or which have anti-tumour vasculature activity, may be useful in methods of therapy. In particular these compounds may be used for treating tumours. As used herein the term “tumour” is used to define any malignant cancerous growth, and may include leukemias, melanomas, colon, lung, ovarian, skin, breast, prostate, CNS, and renal cancers, as well as other cancers.

The compound of the invention having tublin binding activity may also be used in the treatment of solid tumours, eg. breast cancer.

The invention also provides for the use of a compound of formula (I′″″) or (II)-(IV) in the manufacture of a medicament for treating tumours.

There is also provided a method of treatment of solid tumours comprising the administration of an effective amount of a compound of formula (I′″″) or (II)-(IV) to a subject in need thereof.

The compounds of the invention may be particularly useful in combination therapy, eg. combining the treatment with other chemotherapeutic or radiation treatments.

However, it will be understood that the compounds of the invention can be used in the treatment of any disease for which tublin polymerization plays a crucial role.

Compounds of the invention which possess bioactivity, such as tubulin binding activity, can be formulated as a composition, particularly a pharmaceutical composition, together with a pharmaceutically acceptable additive.

The compounds of the invention are administered to the subject in a treatment effective amount. As used herein, a treatment effective amount is intended to include at least partially attaining the desired effect, or delaying the onset of, or inhibiting the progression of, or halting or reversing altogether the onset or progression of the particular disease of condition being treated.

As used herein, the term “effective amount” relates to an amount of compound which, when administered according to a desired dosing regimen, provides the desired therapeutic activity. Dosing may occur at intervals of minutes, hours, days, weeks, months or years or continuously over any one of these periods. Suitable dosages lie within the range of about 0.1 ng per kg of body weight to 1 g per kg of body weight per dosage. The dosage is preferably in the range of 1 μg to 1 g per kg of body weight per dosage, such as is in the range of 1 mg to 1 g per kg of body weight per dosage. In one embodiment, the dosage is in the range of 1 mg to 500 mg per kg of body weight per dosage. In another embodiment, the dosage is in the range of 1 mg to 250 mg per kg of body weight per dosage. In yet another preferred embodiment, the dosage is in the range of 1 mg to 100 mg per kg of body weight per dosage, such as up to 50 mg per body weight per dosage.

Suitable dosage amounts and dosing regimens can be determined by the attending physician and may depend on the particular condition being treated, the severity of the condition as well as the general age, health and weight of the subject.

The active ingredient may be administered in a single dose or a series of doses. While it is possible for the active ingredient to be administered alone, it is preferable to present it as a composition, preferably as a pharmaceutical composition. The formulation of such compositions is well known to those skilled in the art. The composition may contain any suitable carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The carrier must be pharmaceutically “acceptable” in the sense of being compatible with the other ingredients of the composition and not injurious to the subject. Compositions include those suitable for oral, rectal, nasal, topical (including buccal and sublingual), vaginal or parental (including subcutaneous, intramuscular, intravenous and intradermal) administration. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

Compositions of the present invention suitable for oral administration may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g inert diluent, preservative disintegrant (e.g. sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for topical administration in the mouth include lozenges comprising the active ingredient in a flavoured base, usually sucrose and acacia or tragacanth gum; pastilles comprising the active ingredient in an inert basis such as gelatin and glycerin, or sucrose and acacia gum; and mouthwashes comprising the active ingredient in a suitable liquid carrier.

Compositions suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal patches may also be used to administer the compounds of the invention.

Compositions for rectal administration may be presented as a suppository with a suitable base comprising, for example, cocoa butter, glycerin, gelatin or polyethylene glycol.

Compositions suitable for vaginal administration may be presented as pessaries, tampons, creams, gels, pastes, foams or spray formulations containing in addition to the active ingredient such carriers as are known in the art to be appropriate.

Compositions suitable for parenteral administration include aqueous and non-aqueous isotonic sterile injection solutions which may contain anti-oxidants, buffers, bactericides and solutes which render the composition isotonic with the blood of the intended recipient; and aqueous and non-aqueous sterile suspensions which may include suspending agents and thickening agents. The compositions may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage compositions are those containing a daily dose or unit, daily sub-dose, as herein above described, or an appropriate fraction thereof, of the active ingredient.

It should be understood that in addition to the active ingredients particularly mentioned above, the compositions of this invention may include other agents conventional in the art having regard to the type of composition in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

The novel bioactive compounds of the invention can be administered to a subject as a salt or prodrug thereof. The term “salt, or prodrug” includes any pharmaceutically acceptable salt, ester, solvate, hydrate or any other compound which, upon administration to the recipient is capable of providing (directly or indirectly) a compound as described herein. However, it will be appreciated that non-pharmaceutically acceptable salts also fall within the scope of the invention since these may be useful in the preparation of pharmaceutically acceptable salts. Any compound that is a prodrug of a compound of formula (I) is within the scope and spirit of the invention. The term “pro-drug” is used in its broadest sense and encompasses those derivatives that are converted in vivo to the compounds of the invention. Such derivatives would readily occur to those skilled in the art, and include, for example, compounds where a free hydroxy group is converted into an ester, such as an acetate or phosphate ester, or where a free amino group is converted into an amide. Procedures for esterifying, eg. acylating, the compounds of the invention are well known in the art and may include treatment of the compound with an appropriate carboxylic acid, anhydride or chloride in the presence of a suitable catalyst or base. A particularly preferred prodrug is a disodium phosphate ester. The disodium phosphate ester of novel compounds of the invention may be useful in targeting tumour vasculature and thus may provide a means of selective delivery of the compounds to the body. The disodium phosphate ester may be prepared in accordance with the methodology described in Pettit, G. R., et al, Anticancer Drug Des., 1995, 10, 299.

Suitable pharmaceutically acceptable salts include, but are not limited to salts of pharmaceutically acceptable inorganic acids such as hydrochloric, sulphuric, phosphoric nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium. In particular, the present invention includes within its scope cationic salts eg sodium or potassium salts, or alkyl esters (eg methyl, ethyl) of the phosphate group.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

The compounds of the invention may be in crystalline form either as the free compounds or as solvates (e.g. hydrates) and it is intended that both forms are within the scope of the present invention. Methods of solvation are generally known within the art.

It will also be recognised that compounds of the invention may possess asymmetric centres and are therefore capable of existing in more than one stereoisomeric form. The invention thus also relates to compounds in substantially pure isomeric form at one or more asymmetric centres eg., greater than about 90% ee, such as about 95% or 97% ee or greater than 99% ee, as well as mixtures, including racemic mixtures, thereof. Such isomers may be prepared by asymmetric synthesis, for example using chiral intermediates, or mixtures may be resolved by conventional methods, eg., chromatography, or use of a resolving agent.

The synthetic methods and processes described herein are amenable to combinatorial chemistry to produce libraries of compounds for biological screening.

Traditionally, drug candidates have been synthesized individually, this being a time consuming and laborious process if the synthetic sequence contains even just a few steps and large numbers of compounds are to be evaluated for their biological activity. Combinatorial synthesis is an emerging technique for effecting the generation of large libraries of molecules and has been successfully exploited in the synthesis and evaluation of small organic libraries. These libraries and their starting substrates may exist as molecules in free solution or preferably, linked to a solid phase, for example, beads, pins, microtitre plates (wells) or microchips which can be polymeric, glass, silica or other suitable substrate. Chemical diversity can be achieved by either parallel or split (split and mix) syntheses wherein each step has the potential to afford a multitude of compounds. Solution phase libraries may be prepared via parallel syntheses wherein different compounds are synthesised in separate reaction vessels in parallel, often in an automated fashion. Alternatively, attachment of the individual components employed in a synthetic sequence to an appropriate solid phase support allows for the further creation of chemical diversity by utilizing not only parallel synthesis but also split synthesis wherein the solid support containing the compounds prepared in the prior step can be split into a number of batches, treated with the appropriate reagent and recombined.

The substrates can be attached to a solid support surface by any linkers known in the art. The linkers may be any component capable of being cleaved to release the substrate or final compound from the support.

Thus, libraries of compounds can be synthesized by initially attaching the first compound substrate to a solid support surface which can be performed by providing a plurality of solid support surfaces, suitably derivatizing each of the surfaces with groups capable of reacting with either the compound substrate or a linker moiety attached thereto. The various support surfaces with the attached first compound substrate can then be subjected to various reaction conditions and second compound substrates to provide a library of attached compounds, which may, if necessary, be reacted further with third and subsequent compound substrates or varying reactions conditions. Attachment and detachment of substrates and products can be performed under conditions similar to those as described in Johnson, M. G., et al., Tetrahedron, 1999, 55, 11641; Han Y., et al. Tetrahedron 1999, 55, 11669; and Collini, M. D., et al., Tetrahedron Lett., 1997, 58, 7963.

Thus, the invention provides libraries of compounds of at least two compounds of Formula (I), (I′)-(I′″″), (II)-(IV) attached to a solid support surface or pluralities of surfaces.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

General Methods:

Melting points were recorded with a Kofler hot-stage apparatus and are uncorrected. Proton (¹H) and (¹³C) NMR spectra were recorded with a Varian Gemini 300 spectrometer operating at 300 MHz for proton and 75.5 MHz for carbon. All NMR spectra were recorded in (D)chloroform (CDCl₃) at 20° C. The protonicities of the carbon atoms observed in the carbon NMR were determined using attached proton test (APT) experiments. Infrared spectra (IR) we obtained as KBr discs or as films on NaCl plates and were recorded on a Perkin-Elmer Spectrum One Fourier-transform infrared spectrophotometer. Low-resolution electron impact mass spectra (MS) were recorded at 70 eV on either a VG micromass 7070F instrument or a JEOL AX-505H mass spectrometer. High-resolution mass spectra (HRMS) were recorded on a VG micromass 7070F instrument. Elemental analyses were performed on a Carlo Erba 1106. Tetrahydrofuran (THF) was distilled under nitrogen from sodium benzophenone ketyl. Dichloromethane was distilled from calcium hydride. Flash chromatography was performed on Merk Kieselgel 60.

### Indoles

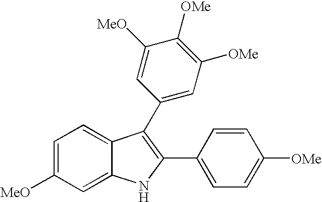

### 6-Methoxy-2-(4-methoxyphenyl)-3-(3,4,5-trimethoxyphenyl)indole (BLF-36-1)

Methylmagnesium chloride (1.37 mL, 3.0 M in THF, 4.11 mmol) was added dropwise to a solution of 2-iodo-5-methoxyacetanilide (550 mg, 2.0 mmol) and 4-methoxyphenyl acetylene (276 mg, 2.1 mmol) in dried THF (5.0 mL) at −5° C. The reaction mixture was then warmed to 18° C., Pd(PPh₃)₂Cl₂ (42 mg, 0.06 mmol) added and the reaction mixture heated to 65° C. for 0.5 h, after which time the reaction was shown to be complete by T.L.C. The solution was cooled to 18° C., diluted with DMSO (8.0 mL) and 3,4,5-trimethoxyiodobenzene (617 mg, 2.1 mmol) added. The solution heated to 80° C. (external temperature) and a slight flow of N₂(g) for 1 h (to remove THF) and heating continued under a stationary atmosphere of N₂(g) for a further 10 h. The reaction mixture was cooled to 18° C. diluted with ethyl acetate (150 mL) and washed with water (2×100 mL) and brine (3×100 mL), dried over MgSO₄ and concentrated onto silica gel (5 g) under reduced pressure. The solid residue was subjected to flash chromatography (silica gel, eluted sequentially with hexane/CH₂Cl₂/diethyl ether 2:1:1 and 1:1:1). The relevant fractions (R_{f}=0.328, CH₂Cl₂) were concentrated giving the product as a white solid (782 mg, 82%).

¹H NMR (300 MHz, CDCl₃) δ 8.19 (br s, 1H), 7.60 (d, J=8.7 Hz, 1H), 7.36 (d, J=8.4 Hz, 2H), 6.91-6.82 (m, 4H), 6.65 (s, 2H), 3.92 (s, 3H), 3.87 (s, 3H), 3.82 (s, 3H), 3.74 (s, 6H). ¹³C NMR+APT (75 MHz, CDCl₃) δ 158.9 (C), 156.6 (C), 153.1 (C), 136.4 (C), 136.2 (C), 132.8 (C), 130.9 (C), 129.2 (CH), 125.2 (C), 123.0 (C), 120.0 (CH), 113.9 (CH), 113.8 (C), 110.1 (CH), 106.8 (CH), 94.4 (CM), 60.9 (CH₃), 55.9 (2×CH₃), 55.7 (CH₃), 55.3 (CH₃). IR (KBr disc, cm⁻¹) 3368, 2999, 2933, 2831, 1574, 1514, 1405, 1332, 1260, 1180, 1128. MS (70 eV) m/z (%): 419 (M⁺, 100), 404 (M⁺—CH₃, 20).

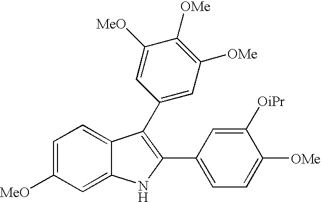

### 6-Methoxy-2-(3-isopropoxy-4-methoxyphenyl)-3-(3,4,5-trimethoxyphenyl)indole

Pd(PPh₃)₂Cl₂ (11 mg, 0.015 mmol) and CuI (6.0 mg, 0.03 mmol) were added to a solution of triethylamine (140 μL, 1.0 mmol), 2-iodo-5-methoxytrifluoroacetanilide (173 mg, 0.50 mmol) and 3-isopropoxy-4-methoxyethynylbenzene (105 mg, 0.55 mmol) in dry acetonitrile (4.0 mL). The reaction mixture was stirred at 18° C. for 1 h, after which time the reaction was shown to be complete by TLC. K₂CO₃ (207 mg, 1.50 mmol) and 3,4,5-trimethoxyiodobenzene 7 (162 mg, 0.55 mmol) were added, and the reaction mixture was stirred at 18° C. for 18 h. After this time it was diluted with diethyl ether (50 mL), washed with H₂O (2×50 mL), dried over MgSO₄ and concentrated onto silica gel (1 g). The solid residue was subjected to flash chromatography (silica gel, hexane/CH₂Cl₂/diethyl ether 3:3:1). The relevant fractions (R_{f}=0.20, in eluant) were concentrated, giving the product as a white solid (183.2 mg, 77%) mp=184-5° C. ¹H NMR (300 MHz, CDCl₃) δ 8.54 (br s, 1H), 7.54 (d, J=8.7 Hz, 1H), 7.04 (d, J=8.7 Hz, 1H), 6.94 (d, J=2.4 Hz, 1H), 6.89-6.81 (m, 3H), 6.68 (s, 2H), 4.24 (septet, J=6.0 Hz, 1H), 3.90 (s, 3H), 3.84 (s, 6H), 3.75 (s, 6H), 1.20 (d, J=6.0 Hz, 6H). ¹³C NMR+APT (75 MHz, CDCl₃) δ 156.5 (C), 153.1 (C), 149.5 (C), 146.8 (C), 136.3 (C), 136.1 (C), 132.8 (C), 131.1 (C), 125.3 (C), 123.1 (C), 119.8 (CH), 115.3 (CH), 113.7 (C), 111.6 (CH), 109.9 (CH), 106.9 (CH), 94.5 (CH), 71.1 (CH), 60.8 (CH₃), 55.9 (CH₃), 55.7 (CH₃), 55.6 (CH₃) 21.7 (CH₃) (2×Ar CH superimposed). IR (KBr disc, cm⁻¹): 3404, 3000, 2930, 2834, 1573, 1496, 1462, 1317, 1249, 1212, 1124. MS (70 eV) m/z (%): 477 (M⁺, 100), 463 (M⁺—CH₃, 13), 420 (12), 351 (20). HRMS calcd for C₂₈H₃₁NO₆: 477.2151. Found: 477.2158.

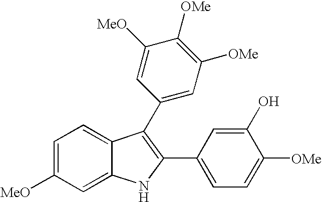

### 6-Methoxy-2-(3-hydroxy-4-methoxyphenyl)-3-(3,4,5-trimethoxyphenyl)indole (BLF-61-3)

Aluminium trichloride (80.0 mg, 0.60 mmol) was added to a solution of isopropylether of BLF-61-3 (above) (96.0 mg, 0.20 nmol) in dry dichloromethane (3 mL) and the reaction mixture stirred at 18° C. for 2 h. The solution was then diluted with NH₄Cl_{(aq)} (sat., 20 mL) and extracted with ethyl acetate (2×15 mL). The combined ethyl acetate extracts were dried over MgSO₄ and concentrated onto silica gel (1 g). The solid residue was subjected to flash chromatography (silica gel, hexane/dichloromethane/diethyl ether 2:2:1), giving the product, BLF-61-3, as a white solid (81.0 mg, 93%) mp=98-9° C. ¹H NMR (300 MHz, D₆DMSO) δ 11.15 (s, 1H), 9.03 (s, 1H), 7.43 (d, J=9.0 Hz, 1H), 6.89 (m, 4H), 6.68 (dd, J=2.1, 9.0 Hz, 1H), 6.57 (s, 2H), 3.77 (s, 3H), 3.76 (s, 3H), 3.69 (s, 3H),3.64 (s, 6H). ¹³C NMR+APT (75 MHz, D₆DMSO) δ 155.9 (C), 152.9 (C), 147.3 (C), 146.3 (C), 136.6 (C), 135.9 (C), 133.2 (C), 131.2 (C), 125.6 (C), 122.4 (C), 119.4 (CH), 115.6 (CH), 112.3 (C), 112.2 (CH), 109.7 (CH), 107.0 (CH), 94.5 (CH), 60.3 (CH₃), 55.8 (CH₃), 55.7 (CH₃), 55.4 (CH₃). IR (KBr disc, cm⁻¹): 3385, 2934, 2833, 1626, 1582, 1513, 1461, 1407, 1338, 1249, 1203, 1161, 1124. MS (70 eV) m/z (%): 435 (M⁺, 100), 420 (M⁺—CH₃, 16). HRMS calcd for C₂₅H₂₅NO₆: 435.1682. Found: 435.1681.

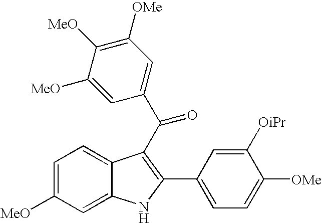

### 6-Methoxy-2-(3-isopropoxy-4-methoxyphenyl)-3-(3,4,5-trimethoxybenzoyl)indole

Pd(PPh₃)₂Cl₂ (11 mg, 0.015 mmol) and CuI (6.0 mg, 0.03 mmol) were added to a solution of triethylamine (140 μL, 1.0 mmol), 2-iodo-5-methoxytrifluoroacetanilide (173 mg, 0.50 mmol) and 3-isopropoxy-4-methoxyethynylbenzene (105 mg, 0.55 mmol) in dry acetonitrile (4.0 mL). The reaction mixture was stirred at 18° C. for 1 h, after which time the reaction was shown to be complete by TLC. K₂CO₃ (207 mg, 1.50 mmol) and 3,4,5-trimethoxyiodobenzene 7 (162 mg, 0.55 mmol) were added and the N₂ (g) atmosphere exchanged for carbon monoxide (1 atm, balloon). This reaction mixture was stirred at 18° C. for 18 h. After this time it was diluted with diethyl ether (50 mL), washed with H₂O (2×50 mL), dried over MgSO₄ and concentrated onto silica gel (1 g). The solid residue was subjected to flash chromatography (silica gel, hexane/CH₂Cl₂/diethyl ether 2:4:1 and 1:1:2). The relevant fractions (R_{f}=0.50, hexane/CH₂Cl₂/diethyl ether 2:1:1) were concentrated, giving the product as a yellow solid (185.0 mg, 73%) mp=196-7° C. ¹H NMR (300 MHz, CDCl₃) δ 9.74 (br s, 1H), 7.93 (d, J=9.0 Hz, 1H), 6.96 (s, 2H), 6.93 (m_{c}, 2H), 6.86 (d, J=2.1 Hz, 1H), 6.73 (d, J=1.8 Hz, 1H), 6.62 (d, J=8.4 Hz, 1H), 4.10 (septet, J=6.0 Hz, 1H), 3.78 (s, 3H), 3.75 (s, 3H), 3.71 (s, 3H), 3.60 (6H), 1.13 (d, J=6.0 Hz, 6H). ¹³C NMR+APT (75 MHz, CDCl₃) δ 192.1 (C), 157.0 (C), 152.3 (C), 150.5 (C), 146.8 (C), 142.9 (C), 140.9 (C), 136.5 (C), 134.5 (C), 124.6 (C), 123.0 (C), 122.1 (CH), 121.1 (CH), 117.0 (CH), 112.1 (C), 111.5 (CH), 111.2 (CH), 107.1 (CH), 94.6 (CH), 71.5 (CH), 60.7 (CH₃), 55.7 (CH₃), 55.6 (CH₃), 55.4 (CH₃) 21.8 (CH₃). IR (KBr disc, cm⁻¹): 3344, 2939, 2835, 1614, 1575, 1541, 1492, 1459, 1420, 1335, 1256, 1203, 1125. MS (70 eV) m/z (%): 505 (M⁺, 100), 463 (M⁺—CH₃CH═CH₂, 26), 308 (26), 218 (46). HRMS calcd for C₂₉H₃₁NO₇: 505.2101. Found: 505.2106.

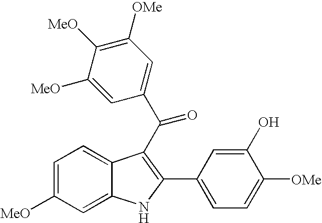

### 6-Methoxy-2-(3-hydroxy-4-methoxyphenyl)-3-(3,4,5-trimethoxybenzoyl)indole (BLF-67-3)

The isopropyl ether of BLF-67-3 (above) was cleaved as described for BLF-61-3 (above) (76.0 mg, 91%) mp=189-90° C. ¹H NMR (300 MHz, CDCl₃) δ 9.04 (br s, 1H), 7.85 (d, J=9.3 Hz, 1H), 6.94 (s, 2H), 6.88 (m, 3H), 6.70 (dd, J=8.4 Hz, 1.8 Hz, 1H), 6.50 (d, J=8.4 Hz), 1H), (d, J=8.7 Hz, 1H), 6.95 (d, J=1.5 Hz, 1H), 6.88-6.82 (m, 3H), 5.69 (s, 1H), 3.82 (s, 3H), 3.79 (s, 3H), 3.75 (s, 3H), 3.67 (s, 6H). ¹³C NMR+APT (75 MHz, CDCl₃) δ 192.5 (C), 156.9 (C), 152.3 (C), 146.9 (C), 145.2 (C), 143.1 (C), 140.8 (C), 136.4 (C), 134.9 (C), 124.9 (C), 122.8 (C), 122.0 (CH), 121.4 (CH), 114.9 (CH), 112.4 (C), 107.1 (CH), 94.5 (CH), 60.7 (CH₃), 55.9 (2×CH₃), 55.7 (CH₃), 55.5 (CH₃). IR (KBr disc, cm⁻¹) 3420, 3322, 2937, 2836, 1626, 1579, 1496, 1455, 1419, 1345, 1321, 1261, 1228, 1199, 1127. MS (70 eV) adz (%): 463 (M⁺, 100), 448 (M⁺—CH₃, 10). HRMS calcd for C₂₅H₂₅NO₇ 463.1631. Found 463.1648.

### Benzofurans and Benzopyrans

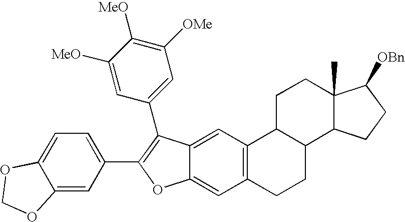

### 2,3-[2′-(3″,4″-methylenedioxyphenyl)-3′-(3′″,4′″,5′″-trimethoxyphenyl)furano]-17-O-benzylestradiol

This product was prepared as described for BLF-36-1 using 2-iodo-17-O-benzylestradiol (270 mg, 0.55 mmol), 3,4-(methylenedioxy)phenylacetylene (104 mg, 0.71 mmol) and 3,4,5-trimethoxyiodobenzene (211 mg, 0.72 mmol) giving white solid product (70% yield)

¹H-NMR (CDCl₃) δ 7.39 (s, 1H, Ar—H), 7.30 (m, 5H, Ar—H), 7.25 (s, 1H, Ar—H), 7.21 (dd, J=8.1 Hz and 1.8 Hz, 1H, Ar—H), 7.16 (d, J=1.8 Hz, 1H, Ar—H), 6.78 (d, J=8.1 Hz, 1H, Ar—H), 6.70 (s, 2H, Ar—H), 5.98 (s, 2H, O—CH₂—O), 4.59 (, 2H, Ph-—(CH₂), 3.97 (s, 3H, OCH₃), 3.83 (s, 6H, 2×OCH₃), 3.53 (t, J=7.8 Hz, 1H, CH), 3.02 (m, 1H), 2.35-0.89 (17H). ¹³C-NMR (CDCl₃) δ 153.8, 152.3, 149.7, 147.7, 147.6 (C—O), 139.4, 137.4, 135.9, 134.4, 130.7, 128.7 (Ar—C), 128.3 (2×Ar—CH), 127.4 (3×ArCH), 124.9 (Ar—C), 121.2 (Ar—CH), 116.4 (Ar—C), 115.9, 110.5, 108.5, 107.3 (Ar—C), 106.7 (2×Ar—C), 101.3 (O—CH₂—O), 88.3 (CH—O-Bn), 71.7 (Ph-—(CH₂), 61.1 (OCH₃), 56.3 (2×OCH₃), 50.5, 44.5, (CH) 43.4 (CH₂), 38.7 (CH), 38.0, 30.2, 28.1, 27.4, 26.7, 23.3 (CH₂), 11.9 (CH₃). LRMS m/z=672 (100) (M⁺), 91 (26) (C₇H₇+).HRMS Calculated=672.308704. Found=672.309253. IR (neat) v max=2932, 1580, 1505, 1489, 1465, 1453, 1411, 1379, 1299, 1236, 1128, 1105, 1038, 736.

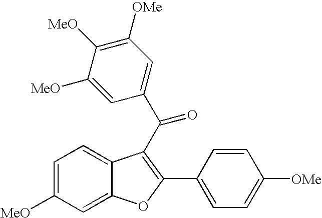

### 6-Methoxy-2-(4′-methoxyphenyl)-3-(3″,4″,5″-trimethoxybenzoyl)benzo[b]furan (BLF-28-1)

Methylmagnesium chloride (1.40 mL, 3.0 M in THF, 4.2 mmol) was added dropwise to a solution of 2-iodo-5-methoxyphenol (500 mg, 2.0 mmol) and 4-methoxyphenyl acetylene (290 mg, 2.2 mmol) in dried THF (5.0 mL) at −5° C. The reaction mixture was then warmed to 18° C., Pd(PPh₃)₂Cl₂ (42 mg, 0.06 mmol) added and the reaction mixture heated to 65° C. for 1 h, after which time the reaction was shown to be complete by T.L.C. The THF was removed by passing a steady flow of N₂ (g) over the heated solution. This was cooled to 18° C. and DMSO (8.0 mL) added and the N₂(g) atmosphere exchanged for carbon monoxide (1 atm.) and stirred for 0.3 h. After this time 3,4,5-trimethoxyiodobenzene (624 mg, 2.12 mmol) was added and the solution heated to 80° C. (external temperature) for 16 h. The reaction mixture was cooled to 18° C. diluted with ethyl acetate (100 mL) and washed with water (2×80 mL) and brine (3×80 mL), dried over MgSO₄ and concentrated onto silica gel (3 g) under reduced pressure. The solid residue was subjected to flash chromatography (silica gel, eluted sequentially with hexane/CH₂Cl₂ 2:1, 1:1, 1:2). The relevant fraction (R_{f}=0.23, hexane/diethyl ether 2:1) were concentrated giving the product as a yellow solid (510 mg, 58%), mp=108-9° C. ¹H NMR (300 MHz, CDCl₃) δ7.56 (d, J=9.0 Hz, 2H), 7.53 (d,J=8.7 Hz, 1H), 7.13 (s, 2H), 7.09 (d, J=2.4 Hz, 1H), 6.92 (dd, J=2.4 Hz, 8.7 Hz, 1H), 6.82 (d, J=9.0 Hz, 2H), 3.89 (s, 3H), 3.87 (s, 3H), 3.79 (s, 3H), 3.70 (s, 6H). ¹³C NMR+APT (75 MHz, CDCl₃) δ 190.4 (C), 160.2 (C), 158.2 (C), 156.9 (C), 154.4 (C), 152.5 (C), 142.0 (C), 132.3 (C), 129.5 (CH), 122.0 (C), 121.5 (C), 121.4 (CH), 114.2 (C), 113.5 (CH), 112.3 (CH), 107.0 (CH), 95.2 (CH), 60.6 (CH₃), 55.7 (2×CH₃), 55.3 (CH₃), 54.9 (CH₃). IR (KBr disc, cm⁻¹) 2935, 2833, 1647, 1609, 1584, 1494, 1455, 1438, 1409, 1368, 1304, 1254. MS (70 eV) m/z (%): 448 (M⁺, 100), 433 (M⁺—CH₃, 15). HRMS calcd for C₂₆H₂₄O₇ 448.1522. Found 448.1520.

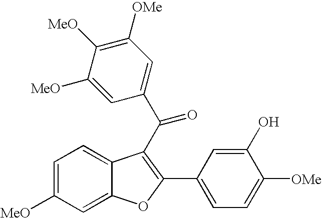

### 6-Methoxy-2-(3-hydroxy-4-methoxyphenyl)-3-(3,4,5-trimethoxybenzoyl)benzo bifuran (BLF-62-3)

The isopropyl ether of BLF-62-3 was prepared using an identical procedure as that described above for BLF-28-1 and the isopropyl ether group cleaved as described for BLF-61-3 above (60% over both steps). BLF-62-3: mp=68-9° C. ¹H NMR (300 MHz, CDCl₃) δ 7.53 (d, J=8.7 Hz, 1H), 7.24 (d, J=2.1 Hz, 1H), 7.13 (s, 2H), 7.10-7.05 (m, 2H), 6.91 (dd, J=8.4 Hz, 2.1 Hz, 1H), 6.72 (d, J=8.4 Hz, 1H), 5.64 (s, 1H), 3.89 (s, 3H), 3.87 (s, 3H), 3.86 (s, 3H), 3.71 (s, 6H). ¹³C NMR+APT (75 MHz, CDCl₃) δ 190.9 (C), 158.4 (C), 156.9 (C), 154.6 (C), 152.7 (C), 147.6 (C), 145.4(C), 142.2 (C), 132.6 (C), 123.0 (C), 121.8 (C), 121.6 (CH), 121.1 (CH), 114.8 (C), 114.1 (CH), 112.6 (CH), 110.2 (CH), 107.2 (CH), 95.5 (CH), 60.8 (CH₃), 56.0 (2×CH₃), 55.8 (CH₃), 55.7 (CH₃). IR (KBr disc, cm⁻¹) 3400, 2940, 2837, 1622, 1581, 1494, 1462, 1414, 1262, 1232, 1126. MS (70 eV) m/z (%): 464 (M⁺, 100), 449 (M⁺—CH₃, 10), 408.2 (15) HRMS calcd for C₂₆H₂₄O₈ 464.1471. Found 464.1459.

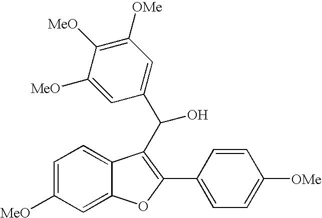

### 6-Methoxy-2-(4-methoxyphenyl)-3-[α-hydroxy-α-3,4,5-trimethoxyphenyl)methyl]benzo[b]furan (BLF-68-3)

Sodium borohydride (15.0 mg, 0.40 mmol) was added to a solution of BLF-28-1 (155 mg, 0.35 mmol) in ethanol (3 mL) and the solution stirred at 18° c for 1 h. The reaction mixture was then diluted with HCl (aq) (1% w/v, 15 mL) and the product extracted with ethyl acetate (2×15 mL). The organic phase was dried over MgSO₄ and concentrated under reduced pressure to give the product as a white solid. This material was suspended in a diethyl ether (3 mL) and filtered giving the pure product as a white solid: mp=53-4° C. ¹H NMR (300 MHz, D₆Acetone) δ 7.77 (d, J=8.7 Hz, 2H), 7.35 (d, J=8.7 Hz, 1H), 7.10-7.05 (m, 3H), 6.81 (s, 2H), 6.74 (dd, J=2.4, 8.7 Hz, 1H), 6.20 (d, J=4.2 Hz, 1H), 5.09 (d, J=4.2 Hz, 1H), 3.85 (s, 3H), 3.82 (s, 3H), 3.70 (s, 6H), 3.68 (s, 3H). ¹³C NMR+APT (75 MHz, D₆Acetone) δ 160.9(C), 158.9 (C), 155.9 (C), 154.1 (C), 152.1 (C), 140.1 (C), 138.9 (C), 129.7 (CH), 124.2 (C), 122.9 (CH), 122.5 (C), 118.5 (C), 115.0 (CH), 112.1 (CH), 104.4 (CH), 96.1 (CH), 68.6 (CH), 60.5 (CH₃), 56.3 (CH₃), 55.9 (CH₃), 55.7 (CH₃). IR (KBr disc, cm⁻¹): 3467, 2936, 2835, 1621, 1592, 1506, 1492, 1461, 1416, 1251, 1150, 1126. MS (70 eV) m/z (%): 450 (M⁺, 100), 281 (11), 149 (16). HRMS calcd for C₂₆H₂₆O₇: 450.1679. Found: 450.1677.

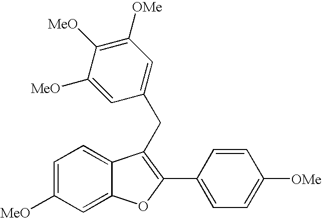

### 6-Methoxy-2-(4-methoxyphenyl)-3-(3,4,5-trimethoxybenzyl)benzo[b]furan (BLF-70-3)

The procedure described above to produce BLF-68-3 was repeated (same scale) and the crude product obtained from extraction with ethyl acetate. The crude product was dissolved in CHCl₃ (3 μL) and triethylsilane (200 μL) and trifluoroacetic acid (300 μL) added and the reaction mixture stirred at 45° C. for 1 h. This solution was then cooled to room temperature, diluted with diethyl ether (30 mL) and NaHCO₃(aq) (sat., 30 mL) added slowly to the swirled solution (gas evolution!). The organic phase was dried over MgSO₄ and concentrated onto silica (1 g) under reduced pressure. The solid residue was subject to flash chromatography (silca gel, sequential elution with hexane/CH₂Cl₂ 2:1, 1:1) giving the product as a white solid: mp=121-2° C. ¹H NMR (300 MHz, CDCl₃) δ 7.66 (d, J=8.7 Hz, 2H), 7.22 (d, J=8.7 Hz, 1H), 7.09 (d, J=3.0 Hz, 1H), 6.98 (d, J=8.7 Hz, 2H), 6.72 (dd, J=3.0 Hz, 8.7 Hz, 1H), 6.49 (s, 2H), 4.14 (s, 2H), 3.87 (s, 3H), 3.85 (s, 3H), 3.84 (s, 3H), 3.75 (s, 6H). ¹³C NMR+APT (75 MHz, CDCl₃) δ 159.4 (C), 157.9 (C), 154.8 (C), 153.3 (C), 151.4 (C), 136.3 (C), 135.2 (C), 127.9 (CH), 123.9 (C), 123.7 (C), 119.7 (CH), 114.2 (CH), 112.0 (C), 111.3 (CH), 104.9 (CH), 95.7 (CH), 60.8 (CH₃), 56.0 (2×CH₃), 55.7 (CH₃), 55.3 (CH₃), 30.3 (CH₂). IR (KBr disc, cm⁻¹) 2935, 2835, 1613, 1587, 1491, 1506, 1460, 1437, 1330, 1243, 1181, 1163, 1138. MS(70 eV)m/z (%):434(M⁺, 100),419 (M⁺—CH₃, 11). HRMS calcd for C₂₆H₂₆O₆ 434.1729. Found 434.1740.

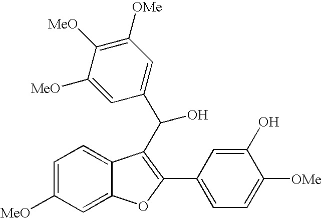

### 6-Methoxy-2-(3-hydroxy-4-methoxyphenyl)-3-[α-hydroxy-α-(3,4,5-trimethoxyphenyl)methyl]benzo[b]furan (BLF-69-3):

This product was prepared in a similar fashion to BLF-68-3 using BLF-62-3 (95 mg, 0.2 mmol) and sodium borohydride (33 mg, 1.0 mmol). The product was obtained as a white solid (90 mg, 94%): mp=68-9° C. ¹H NMR (300 MHz, d₆Acetone) δ 8.00 (s, 1H), 7.35 (d, J=8.7 Hz, 1H), 7.33 (d, J=2.1 Hz, 1H), 7.28 (dd, J=2.1 Hz, 8.7 Hz, 1H), 7.07 (m, 2H), 6.82 (s, 1H), 6.73 (dd, J=8.7 Hz, 2.1 Hz, 1H), 6.21 (d, J=4.5 Hz, 1H), 5.08 (d, J=4.5 Hz, 1H), 389 (s, 3H), 3.82 (s, 3H), 3.71 (s, 6H), 3.68 (s, 3H). ¹³C NMR+APT (75 MHz, d₆Acetone) δ 158.8 (C), 155.8 (C), 154.1 (C), 152.1 (C), 148.9 (C), 147.6 (C), 138.0(C), 124.7(C), 123.0 (CH), 122,4 (C), 120.0 (CH), 118.6 (C), 115.0 (CH), 112.5 (CH), 112.0 (C), 104.4 (CH), 96.1 (CH), 68.6 (CH), 60.4 (CH₃), 56.3 (3×CH₃), 55.8 (CH₃). IR (KBr disc, cm⁻¹) 3435, 2936, 2836, 1623, 1591, 1507, 1493, 1461, 1260, 1232, 1125. MS (70 eV) m/z (%): 466 (M⁺, 100), 448 (M⁺—H₂O, 18), 408 (98). HRMS calcd for C₂₆H₂₆O₈ 466.1628. Found 466.1630.

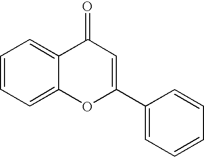

### 2-Phenylbenzo[b]pyran-4-one

n-Butyllithium (0.56 mL, 1.0 mmol) was added to a solution of 2-iodophenol (109.9 mg, 0.500 mmol) in THF (2 mL) at 78° C. (dry-ice/acetone bath). After 0.1 h, 3-phenylpropynoyl chloride (82.2 mg, 0.500 mmol) was added, and the reaction warmed to room temperature. After 1 h, DMSO (4 mL) and water (0.5 mL) were added. After further stirring for 1 h, the solution was diluted with diethyl ether (50 mL), washed with NH₄Cl(aq) (10%, 40 mL), water (40 mL), dried over MgSO₄, and concentrated under reduced pressure onto silica gel (2 g). The residue was subjected to flash chromatography (silica gel, hexane/diethyl ether 95:5, 4:1, then 1:1) to give the product as a white solid (86 mg, 78%).

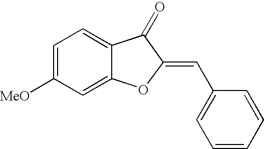

### 2-benzylidene-6-methoxybenzo[b]furan-3-one

n-Butyllithium (0.86 mL, 1.72 mmol) was added to a solution of 2-iodo-5-methoxyphenol (200 mg, 0.858 mmol) in THF (4 mL) at −78° C. (dry-ice/acetone bath). After 0.15 h, 3-phenylpropyroyl chloride (142 mg, 0.858 mmol) was added, and the reaction warmed to room temperature and quenched with NH₄Cl_{(aq)} (10%, 30 mL). The solution was extracted with diethyl ether (3×50 mL), and concentrated under reduced pressure to give a brown residue. The residue was diluted with methanol (6 mL), and AgNO₃ (90 mg, 0.515 mmol) added, and the solution stirred for 2 h. After this time, the solution was diluted with diethyl ether (50 mL), washed with water (40 mL), dried over MgSO₄, and concentrated under reduced pressure onto silica gel (2 g). The residue was subjected to flash chromatography (silica gel, hexane/diethyl ether 9:1, 5:1, then 1:1) to give the product as a white solid (159, 74%).

### Indanones and Indenones

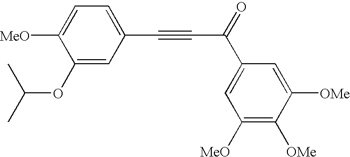

### 3-(3′-Isopropoxy-4′-methoxyphenyl)-1-(3″,4″,5″-trimethoxyphenyl)propyn-1-one

n-Butyllithium (9.4 mL, 2.00 M in hexanes, 19 mmol) was added dropwise to a solution β,β-dibromo-3-isopropoxy-4-methoxystyrene (3.29 g, 9.39 mmol) in dry THF (50 mL) at −78° C. (dry ice/acetone) (produces lithium 3-isopropoxy-4-methoxyphenylacetylide). The solution was then allowed to return to room temperature before being again cooled to −78° C. 3,4,5-Trimethoxybenzoyl chloride [2.27 g, 9.86 mmol, dissolved in THF (20 mL)] was added and the solution once again allowed to warn to room temperature. The reaction mixture was diluted with diethyl ether (100 mL), washed with distilled water (2×50 mL), dried over MgSO₄ and concentrated onto silica gel (1.5 g) under reduced pressure. The solid residue was subject to flash chromatography (silica gel, 2% ether in dichloromethane) giving a white solid (2.27 g, 65.7%), mp=130-31° C. ¹H NMR (300 MHz, CDCl₃) δ 7.50 (s, 2H, Ar—H), 7.28 (dd, J=8.3 Hz, 1.8 Hz, 1H, Ar—H), 7.16 (d, J=1.8 Hz, 1H, Ar—H), 6.89 (d, J=8.3 Hz, 1H, Ar—H), 4.54 (m, J=6.1 Hz, 1H, i-Pr), 3.96 (s, 6H, OMe), 3.94 (s, 3H, OMe), 3.90 (s, 3H, OMe), 1.38 (d, 6H, J=6.1 Hz, i-Pr).¹³C NMR+APT (75 MHz, CDCl₃) δ 176.8 (C), 153.0 (C), 152.9 (C), 147.1 (C), 143.3 (C), 132.3 (C), 127.3 (CH), 119.3 (CH), 111.7 (C), 111.6 (CH), 106.7 (CH), 94.4 (C), 86.3.4 (C), 71.6 (CH), 61.0 (CH₃), 56.2 (CH₃), 56.0 (CH₃), 21.9 (CH₃). IR (KBr disc, cm⁻¹) 3011, 2976, 2938, 2838, 2187, 1637, 1586, 1510, 1460, 1414. MS (70 eV) m/z (%): 384.1(M⁺), 342.0, 299.0, 175.0

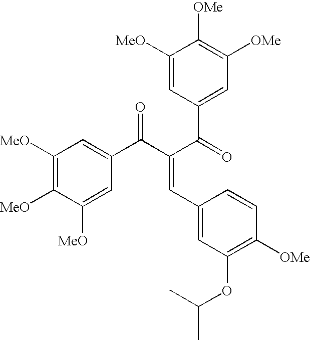

### 2-(3′-Isopropoxy-4′-methoxybenzylidine)-1,3-bis-(3″,4′,5′-trimethoxyphenyl)propan-1,3-dione

Bis(dibenzylidineacetone)palladium (16 mg, 0.03 mmol) and triphenylphosphine (30 mg, 0.12 mmol) were dissolved in dry tetrahydrofuran (8 mL) under N₂ and stirred until the solution changed from red to yellow/orange (approximately 1 h). To this solution was added 1,3-diarylpropynone above (0.384 g, 1.00 mmol), followed by dropwise addition of tributyltin hydride (0.28 mL, 1.0 mmol). The solution was then allowed to stir until TLC indicated complete reaction of the starting alkyne (approximately 0.5 h), after this time 3,4,5-trimethoxybenzoyl chloride (0.242 g, 1.05 mmol) and cuprous chloride (0.08 g, 0.80 mmol) were added. The solution was then stirred until TLC revealed complete consumption of the 3,4,5-trimethoxybenzoyl chloride (approximately 3 h). After this time the THF solution was taken up in diethyl ether (100 mL) and washed with aqueous KF solution (30%, 3×50 mL). The organic phase was then dried over MgSO₄ and concentrated onto silica gel (1 g) under reduced pressure. The solid residue was subject to flash chromatography (silica gel, 4% diethyl ether in dichloromethane) giving a light yellow solid (0.475 g, 81.7%), mp=105-6° C. ¹H NMR (300 MHz, CDCl₃) δ 7.56 (s, 1H, C═C—H), 7.28 (s, 2H, Ar—H), 7.10 (s, 2H, Ar—H), 6.97 (dd, J=8.5, 2.0 Hz, 1H, Ar—H), 6.86 (d, J=2.0 Hz, 1H, Ar—H), 6.78 (d, J=8.5 Hz, 1H, Ar—H), 4.19 (m, J=6.1 Hz, 1H, i-Pr), 3.91 (s, 3H, OMe), 3.88 (s, 3H, OMe), 3.83 (s, 9H, OMe), 3.80 (s, 6H, OMe), 1.18 (d, J=6.1 Hz, 6H, i-Pr).).¹³C NMR+APT (75 MHz, CDCl₃) δ 196.0 (C), 193.4 (C), 153.2 (C), 152.9 (C), 152.4 (C), 147.1 (C), 143.3 (CH), 143.2 (C), 141.8 (C), 136.7 (C), 132.6 (C), 131.3 (C), 125.7 (C), 125.2 (CH), 116.0 (CH), 111.3 (CH), 106.8 (CH), 106.7(CH), 71.3 (CH), 60.9 (CH₃), 56.1 (CH₃), 55.8 (CH₃), 21.7 (CH₃). IR (KBr disc, cm⁻¹) 2971, 2940, 2836, 1646, 1578, 1504, 1122, 998. MS (70 eV) m/z (%): 580.1(M⁺), 385.1, 327.0, 195.0, 152.0, 77.0.

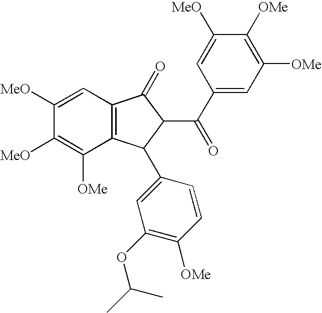

### 3-(3′-isopropoxy-4′-methoxyphenyl)4,5,6-trimethoxy-2-(3″,4″,5″-trimethoxybenzoyl)-1-indanone

1,3-Propadione above (0.581 g, 1.00 mmol) was dissolved in dry dichloromethane (20 mL), to this solution was added methanesulfonic acid (68 μL, 1.0 mmol). After 1 h stirring at room temperature the solution was taken up in diethyl ether (50 mL) and washed with distilled water (2×20 mL), the organic phase was then dried over MgSO₄ and the solvent removed under reduced pressure, a light yellow solid was returned (0.576 g, 99.1%), mp=65-6° C. The ¹H NMR spectrum of this compound indicates the presence of an equilibrium mixture of the trans-isomer and an enol tautomer. ¹H NMR (300 MHz, CDCl₃) δ 7.26 (s), 7.19 (s), 7.05 (s), 6.83 (s), 6.79 (d), 6.68 (dd), 6.67 (s), 6.64 (d), 6.61 (s), 6.56 (d), 5.14 (s), 4.99 (d, J=2.5 Hz), 4.57 (d, J=2.5 Hz), 4.42 (m), 4.29 (m), 3.71-3.95 (10 singlets), 3.45 (s), 3.32 (s), 1.31 (d), 1.26 (d), 1.20 (d), 1.18 (d).MS (70 eV) m/z (%): 580.1(M⁺), 385.1, 343.0, 195.0. IR (KBr disc, cm⁻¹) 2972 m. 2937 m, 2835 w, 1714 m, 1667 m, 1583 s, 1505 s, 1336 s, 1125 vs

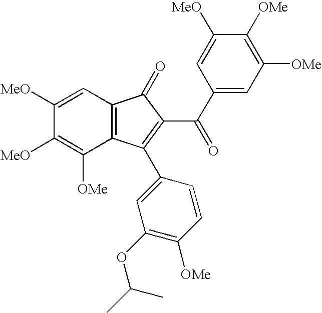

### 3-(3′-isopropoxy-4′-methoxyphenyl)-4,5,6-trimethoxy-2-(3″,4″,5″-trimethoxybenzoyl)indenone

Indanone above (0.232 g, 0.40 mmol) and 2,3-dichloro-5,6-dicyanoquione (0.136 g, 0.60 mmol) were dissolved in dry 1,2-dichloroethane (5 mL) and stirred at 60° C. for 3 days. After this time the solution was decanted from the precipitate (dihydro-DDQ), concentrated onto silica gel (0.5 g) and chromatographed (2:2:1 hexanes/dichloromethane/diethyl ether) to return a bright orange solid (185 mg, 80.1%), mp=170-171° C. ¹H NMR (300 MHz, CDCl₃) δ 7.16 (dd, J=8.4, 2.1 Hz, 1H, Ar—H), 7.11 (s, 1H, Ar—H), 7.03 (s, 2H, Ar—H), 7.02 (d, J=2.1 Hz, 11H, Ar—H), 6.76 (d, J=8.4 Hz, 1H, Ar—H), 4.35 (m, J=6.1 Hz, 1H, i-Pr), 3.94 (s, 3H, OMe), 3.92 (s, 3H, OMe), 3.84 (s, 3H, OMe), 3.82 (s, 3H, OMe), 3.77 (s, 6H, OMe), 3.39 (s, 3H, OMe), 1.25 (d, J=6.1 Hz, 6H, i-Pr). ¹³C NMR+APT (75 MHz, CDCl₃) δ 192.1 (C), 191.2 (C), 161.8 (C), 155.5 (C), 152.7 (C), 152.1 (C), 150.6 (C), 147.4 (C), 146.3 (C), 142.7 (C), 131.8 (C), 131.4 (C), 127.4 (C), 126.2, 125.0 (C), 122.2 (CH), 116.0 (CH), 110.5 (CH), 106.9 (CH), 105.0 (CH), 71.5 (CH), 61.6 (CH₃), 61.2 (CH₃), 60.8 (CH₃), 56.5 (CH₃), 56.1 (CH₃), 55.8 (CH₃), 21.9 (CH₃). IR (KBr disc, cm⁻¹)3001, 2974, 2937, 2839, 1709, 1641, 1600, 1583, 1507, 1460, 1416, MS(70 eV)m/z(%): 578.0(M#), 534.9, 368.9, 195.0

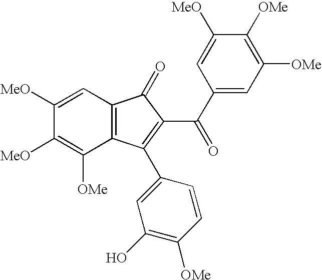

### 3-(3′-Hydroxy-4′-methoxyphenyl)-4,5,6-trimethoxy-2-(3′,4″,5″-trimethoxybenzoyl)indenone (DK-12a-1)

Indenone above (101 mg, 0.175 mmol) was dissolved in 5 mL of dry dichloromethane, to this solution was added aluminium trichloride (94 mg, 0.700 mmol). After stirring for 0.15 h the mixture was taken up in diethyl ether (20 mL) and washed with aqueous ammonium chloride solution (10%, 2×30 mL), and water (20 mL). The organic phase was dried over MgSO₄ and evaporated onto silica gel (0.5 g) under reduced pressure. The solid residue was subject to flash chromatography (silca gel, 7% diethyl ether in dichloromethane) giving an orange solid (71.4 mg, 76.1%). ¹H NMR (300 MHz, CDCl₃) δ7.11 (d, J=2.1 Hz, 1H, Ar—H), 7.10 (s, 1H, Ar—H), 7.01 (s, 2H, Ar—H), 6.96 (dd, J=7.8, 2.1 Hz, 1H, Ar—H), 6.67 (d, J≈7.8 Hz, 1H, Ar—H), 5.57 (s, 1H, OH), 3.94 (s, 3H, Ome), 3.91 (s, 3H, Ome), 3.85 (s, 3H, Ome), 3.84 (s, 3H, Ome), 3.79 (s, 6H, Ome), 3.48 (s, 3H, Ome). ¹³C NMR+APT (75 MHz, CDCl₃) δ 192.1 (C), 191.0 (C), 162.1 (C), 155.7 (C), 152.7 (C), 150.7 (C), 148.1 (C), 147.4 (C), 144.8 (C), 142.5 (C), 132.0 (C), 131.5 (C), 127.3 (C), 126.2, 126.0 (C), 121.1 (CH), 114.7 (CH), 109.5 (CH), 106.8 (CH), 104.9 (CH), 61.4 (CH₃), 61.2 (CH₃), 60.8 (CH₃), 56.6 (CH₃), 56.1 (CH₃), 55.8 (CH₃). IR (KBr disc, cm⁻¹) 3418, 2934, 2840, 1707, 1641, 1606, 1581, 1504, 1465, 1411, 1362, 1339, 1124. MS (70 eV) m/z (%): 536.0(M⁺),493.0, 369.0, 343.0, 277.0, 219.0, 195.0

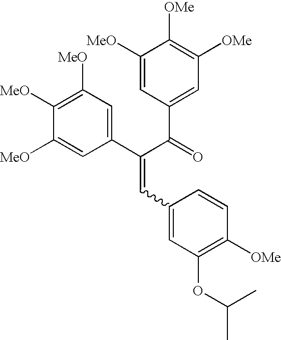

### 3-(3′-Isopropoxy-4′ methoxyphenyl)-1,2-bis(3″,4″,5″-trimethoxyphenyl)propen-1-one

Bis(dibenzylidineacetone)pailadium (13 mg, 0.024 mmol) and triphenylphosphine (24 mg, 0.10 mmol) were dissolved in dry tetrahydrofuran (8 mL) under N₂ and stirred until the solution changed from red to yellow/orange (approximately 1 h). To this solution was added propynone above (0.307 g, 0.800 mmol), followed by dropwise addition of tributyltin hydride (97% solution, 0.22 mL, 0.800 mmol). The solution was then allowed to stir for 5 hours, after this time 3,4,5-trimethoxyiodobenene (0.194 g, 0.84 mmol) and cuprous chloride (0.16 g, 1.6 mmol) were added. The solution was then stirred until TLC revealed complete consumption of the 3,4,5-trimethoxyiodobenzene (3 days). After this time the THF solution was taken up in diethyl ether (100 mL) and washed with aqueous KF solution (30%, 3×50 mL). The organic phase was then dried over MgSO₄ and evaporated under reduced pressure onto silica gel (1 g). Flash chromatography (sequential elution 2%/4% diethyl ether in dichloromethane) returned two isomers (higher R_{f}, 0.024 g, 5.4%/lower R_{f}, 0.105 g, 23.7%).

### Higher R_{f} Isomer

¹H NMR. δ7.31 (s, 2H, Ar), 7.04 (s, 1H, C═C—H), 6.87 (dd, J=8.2, 2.1 Hz, 1H, Ar), 6.79 (d, J=2.1 Hz, 1H, Ar), 6.73 (d, J=8.2, Hz, 1H, Ar), 6.64 (s, 2H, Ar), 4.22 (m, J=6.1 Hz, 1H, i-Pr), 3.88 (s, 3H, OMe), 3.85 (s, 3H, OMe), 3.84 (s, 6H, OMe), 3.80 (s, 9H, 2×OMe), 1.20 (d, J=6.1 Hz, 6H, i-Pr).

### Lower R_{f} Isomer

¹H NMR: δ 7.26 (s, 1H, C═C—H), 7.09 (s, 2H, Ar), 6.83 (dd, J=8.4, 2.1 Hz,1H, Ar), 6.76 (d, J=8.4 Hz, 1H, Ar), 6.63 (d, J=2.1, Hz, 1H, Ar), 6.51 (s, 2H, Ar), 4.04 (m, J=6.1 Hz, 1H, i-Pr), 3.92 (s, 3H, OMe), 3.86 (s, 9H, 2×OMe), 3.83 (s, 3H, OMe), 3.77 (s, 6H, OMe), 1.18 (d, J=6.1 Hz, 6H, i-Pr).

APT ¹³C NMR: δ 196.0 (C), 153.7 (C), 152.7 (C), 151.3 (C), 146.6 (C), 141.4 (C), 141.1 (CH), 137.8 (C), 137.4 (C), 133.3 (C), 132.6 (C), 127.0 (C), 125.4 (CH), 115.6 (CH), 110.9 (CH), 107.1 (CH), 106.4 (CH), 70.8 (CH), 60.8 (CH₃), 60.7 (CH₃), 56.2 (CH₃), 56.0 (CH₃), 55.7 (CH₃), 21.7 (CH₃).

LRMS: m/z 552.2 (M⁺), 509.2, 479.2, 345.1, 303.1, 269.0, 195.0

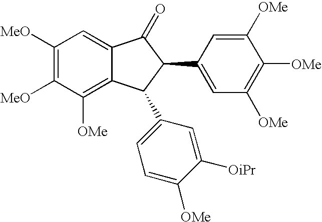

### (±)trans-3-(3′-Isopropoxy-4′-methoxyphenyl)-4,5,6-trimethoxy-2-(3″,4″,5″-trimethoxyphenyl)-1-indanone

Prepared from 3-(3′-isopropoxy-4′methoxyphenyl)-1,2-bis(3″,4″,5″-trimethoxyphenyl)propen-1-one (above) in a similar manner as described for 3-(3′-isopropoxy-4′-methoxyphenyl)-4,5,6-trimethoxy-2-(3′,4″,5″-trimethoxybenzoyl)-1-indanone (above), giving the product as tan solid (80.8 mg, 81.0%): ¹H NMR (300 MHz, CDCl₃) δ 7.15 (s, 1H, Ar), 6.79 (d, J=8.0 Hz, 1H, Ar), 6.63 (dd, J=8.0, 2.1 Hz, 1H, Ar), 6.61 (d, J=2.1 Hz, 1H, Ar), 6.24 (s, 2H, Ar), 4.41 (m, J=6.1 Hz, 1H, i-Pr), 4.41 (d, J=3.3 Hz, 1H, methine), 3.93 (s, 3H, OMe), 3.92 (s, 3H, OMe), 3.81 (s, 3H, OMe), 3.80 (s, 3H, OMe), 3.75 (s, 6H, OMe), 3.61 (d, J=3.3 Hz, 1H, methine) 3.38 (s, 3H, OMe), 1.30 (d, J=6.1 Hz, 3H, i-Pr), 1.26 (d, J=6.1 Hz, 3H, i-Pr).¹³C NMR+APT (75 MHz, CDCl₃) δ 204.5 (C), 155.1 (C), 153.4 (C), 150.2 (C), 149.3 (C), 149.1 (C), 147.1 (C), 143.0 (C), 137.0 (C), 135.9 (C), 134.7 (C), 131.5 (C), 119.8 (CH), 115.4 (CH), 111.9 (CH), 104.9 (CH), 100.8 (CH), 71.3 (CH), 65.0 (CH), 60.8 (CH₃), 60.7 (CH₃), 60.1 (CH₃), 56.2 (CH₃), 56.0 (CH₃), 55.9 (CH₃), 22.0 (CH₃), 21.8 (CH₃).

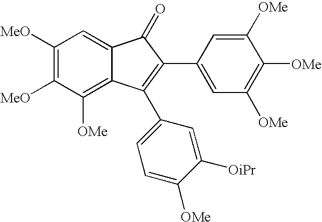

### (±)trans-3-(3′-Isopropoxy-4′-methoxyphenyl)-4,5,6-trimethoxy-2-(3″,4″,5″-trimethoxyphenyl)-1-indenone

(±)trans-3-(3′-Isopropoxy-4′-methoxyphenyl)-4,5,6-trimethoxy-2-(3″,4″,5″-trimethoxyphenyl)-1-indanone (0.0801 g, 0.145 mmol) and DDQ (50 mg, 0.22 mmol) were dissolved in dry 1.2-dichloroethane (5 mL) and stirred at 80° C. for 12 h. After this time the reaction mixture was evaporated onto silica gel (0.5 g) and flash chromatographed (7:7:1 hexanes/dichloromethane/diethyl ether) to return a red solid (42.6 mg, 53.4%).:

¹H NMR (300 MHz, CDCl₃) δ 7.06 (s, 1H, Ar), 7.02 (dd, J=8.4, 1.9 Hz, 1H, Ar), 6.91 (d, J=1.9 Ti., 1H, Ar), 6.87 (d, J=8.4 Hz, 1H, Ar), 6.44 (s, 2H, Ar), 4.37 (m, J=6.1 Hz, 1H, i-Pr), 3.91 (s, 3H, OMe), 3.88 (s, 3H, OMe), 3.87 (s, 3H, OMe), 3.78 (s, 3H, OMe), 3.62 (s, 6H, OMe), 3.33 (s, 3H, OMe), 1.24 (d, J=6.1 Hz, 3H, i-Pr). ¹³C NMR+APT (75 MHz, CDCl₃) δ 195.6 (C), 152.7 (C), 154.3 (C), 152.7 (C), 150.8 (C), 147.7 (C), 146.6 (C), 137.3 (C), 131.4 (C), 130.2 (C), 128.6 (C), 126.9 (C), 126.7 (C), 126.6 (C), 122.0 (CH), 116.5 (CH), 111.2 (CH), 107.2 (CH), 104.8 (CH), 71.4 (CH), 61.5 (CH₃), 61.2 (CH₃), 60.8 (CH₃), 56.6 (CH₃), 56.0 (CH₃), 55.8 (CH₃), 21.8 (CH₃).

### Thiophenes and Benzothiophenes

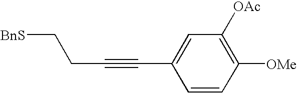

### 4-[3′-acetoxy-4′-methoxyphenyl]-3-butynyl sulfide

CuI (22 mg, 0.12 mmol) was added to a solution of benzyl 3-butynyl sulfide (502 mg, 2.85 mmol), 3-acetoxy-4-methoxy-iodobenzene (643 mg, 2.20 mmol) and Pd(PPh₃)₂Cl₂ (40 mg, 0.06 mmol) in a solution of DMF (3 mL) and Et₃N (1 mL). The resultant solution was stirred for 16 h at 18° C. The resultant solution was diluted diethyl ether (30 mL) and washed with HClaq (1% in H₂O, 30 mL) and water (3×30 mL), dried over MgSO₄ and concentrated on to silica gel (2 g) under reduced pressure. The solid residue was subject to flash chromatography (silica gel, hexane/diethyl ether 9:1) giving the product as a viscous oil (734 mg, 98%). ¹H NMR (300 MHz, CDCl₃) δ 7.41-7.39 (m, 5H), 7.28 (dd, J=2.1, 8.4 Hz, 1H), 6.27 (d, J=2.1 Hz, 1H), 6.87 (d, J=8.4 Hz, 1H), 3.83 (s, 2H), 3.82 (s, 3H), 2.68 (s, 4H), 2.32 (s, 3H). ¹³C NMR+APT (75 MHz, CDCl₃) δ 168.4 (C), 150.9 (C), 139.2 (C), 138.0 (C), 130.2 (CH), 128.7 (CH), 128.3 (CH), 126.8 (CH), 125.8 (CH), 115.8 (C), 112.0 (CH), 87.2 (C), 80.4 (C), 55.7 (CH₃), 36.1 (CH₂), 30.1 (CH₂), 20.5 (CH₂), 20.3 (CH₃). IR (NaCl film, cm⁻¹) 3026, 2922, 2839, 1766, 1509, 1368, 1296, 1268, 1202, 1125. MS (70 eV) m/z (%): 340 (M⁺, 22), 298 (M⁺—CH.CO, 65), 161 (40), 91 (100). HRMS calcd for C₂₀H₂₀O₃S 340.1133. Found 340.1129.

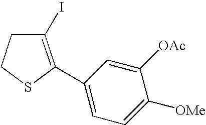

### 2-(3′-acetoxy-4′-methoxyphenyl)-4,5-dihydro-3-iodothiophene

Iodine (224 mg, 0.88 mmol) was added to a solution of benzyl 4-(3′-acetoxy-4′-methoxyphenyl)-3-butynyl sulfide (300 mg, 0.88 mmol) in CH₂Cl₂ (3 mL) and the reaction mixture stirred at 18° C. for 0.15 h. After this time the solution was diluted with diethyl ether (15 mL) and was washed with Na₂S₂O₅ (5% w/v, 15 mL) and water (15 mL), dried over MgSO₄ and concentrated onto silica gel (1 g). The solid residue loaded onto a short column of silica gel (5 cm×1.5 cm) and eluted sequently with hexane/diethyl ether 9:1 and 8:2 giving the product as a white solid (328 mg, 99%) mp=81-3° C. ¹H NMR (300 MHz, CDCl₃) δ 7.45 (dd, J=2.1, 8.4 Hz, 1H), 7.29 (d, J=2.1 Hz, 1H), 6.95 (d, J=8.4 Hz, 1H), 3.84 (s, 3H), 3.40-3.20 (m_{c}, 4H), 2.32 (s, 3H). ¹³C NMR+APT (75.5 MHz, CDCl₃) δ 168.6 (C), 151.1 (C), 138.9 (C), 138.9 (C), 127.4 (CH), 127.1 (C), 123.3 (CH), 111.6 (CH), 73.6 (C), 55.8 (CH₃), 49.4 (CH₂), 32.0 (CH₂) 20.5 (CH₃). IR (KBr disc, cm⁻¹) 2924, 2832, 1760, 1603, 1507, 1366, 1270, 1208. MS (70 eV) m/z (%): 376 (M⁺, 72), 334 (M⁺—CH₂CO, 100), 161 (41). HRMS calcd for C₁₃H₁₃O₃SI 375.9630. Found 375.9633.

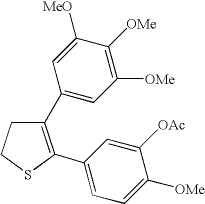

### 2-(3′-acetoxy-4′-methoxyphenyl)-4,5-dihydro-3-(3″,4″,5″-trimethoxyphenyl)thiophene (GPF-60-1)

t-Butyllithium (0.36 mL, 1.7 M in hexanes, 0.61 mmol) was added to a solution 3,4,5-trimethoxyiodobenzene (88 mg, 0.30 mmol) in THF (3 mL) at −78° C. (dry-ice/acetone). Zinc chloride (42 mg, 0.31 mmol) was added and the reaction mixture warmed to room temperature. Pd(PPh₃)₂Cl₂, (7.0 mg, 0.01 mmol) and 2-(3′-acetoxy-4′-methoxyphenyl)-4,5-dihydro-3-iodothiophene (77 mg, 0.20 mmol) were added and the resultant solution stirred at room temperature for 6 h. Methanol (2 mL) and K₂CO₃ (140 mg, 1.01 mmol) were added to the reaction mixture and stirring continued for a further 1 h. The reaction mixture was diluted with NH₄Cl_{(aq)} (sat., 40 mL) and extracted with diethyl ether (50 mL), dreid over MgSO₄ and concentratede onto silica gel (1 g). The solid isidue was subjected to flash chromatography (silica gel, hexane/diethyl ether 2:1, 1:1) giving the product as white solid (63 mg, 82%). ¹H NMR (300 MHz, CDCl₃) δ 6.94 (d, J=2.1 Hz, 1H), 6.80 (dd, J=2.1, 8.4 Hz, 1H), 6.73 (d, J=8.4 Hz, 1H), 6.34 (s, 2H), 3.87 (s, 3H), 3.83 (s, 3H), 3.64 (s, 6H), 3.34 (m., 4H), 2.32 (s, 3H). ¹³C NMR+APT (75.5 MHz, CDCl₃) δ 168.6 (C), 151.1 (C), 138.9 (C), 138.9 (C), 127.4 (CH), 127.1 (C), 123.3 (CH), 111.6 (CH), 73.6 (C), 55.8 (CH₃), 49.4 (CH₂), 32.0 (CH₂) 20.5 (CH₃). IR (KBr disc, cm⁻¹) 2924, 2832, 1760, 1603, 1507, 1366, 1270, 1208. MS (70 eV) m/z (%): 376 (, 72), 334 (le —CH₂CO, 100), 161 (41). HRMS calcd for C₁₃H₁₄O₃SI 375.9630. Found 375.9633.

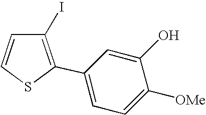

2,3-Dichloro-5,6-dicyano-1,4-benzoquinone (272 mg, 1.2 mmol) was added to a solution of 2-(3′-acetoxy-4′-methoxyphenyl)-4,5-dihydro-3-iodothiophene (376 mg, 1.0 mmol) in dichloromethane (4 mL). After stirring at 18° C. for 1 h the reaction mixture was concentrated onto silica gel (2 g) and the solid residue subjected to flash chromatograph (silica gel, hexane/diethyl ether 1:1) the product was obtained as a viscous resin (363 mg, 95%). This material was not fully characterized: ¹H NMR (300 MHz, CDCl₃) δ 7.48 (dd, J=2.1, 8.4 Hz, 1H), 7.35 (d, J=2.1 Hz, 1H), 7.26 (d, J=5.1 Hz, 1H), 7.12 (d, J=5.1 Hz, 1H),7.03 (d,J=8.4 Hz, 1H), 3.89(s,3H),2.34(s, 3H).

The resin was dissolved in methanol (6 mL) and K₂CO₃ (690 mg, 5.0 mmol) added and the resultant slurry stirred at 18° C. for 1 h. The reaction mixture was then diluted with NH₄Cl_{(aq)} (sat. 50 mL) and extrated with diethyl ether (2×50 mL). The combined organic fractions were dried over MgSO₄ and concentrated onto silica gel (2 g). The solid residue was subject to flash chromatography (silica gel, hexane/diethyl ether 2:1, 1:1) and the product obtained as a viscous oil (302 mg, 91% from 2-(3′-acetoxy-4′-methoxyphenyl)-4,5-dihydro-3-iodothiophene). ¹H NMR (300 MHz, CDCl₃) δ 7.25 (d, J=5.4 Hz, 1H), 7.22 (d,J=2.1 Hz, 1H), 7.15-7.10 (m, 2H), 6.92 (d,J=8.4 Hz, 1H), 5.77 (brs, 1H), 3.94 (s, 3H). ¹³C NMR+APT (75.5 MHz, CDCl₃) δ 146.7 (C), 145.3 (C), 142.1 (C), 136.3 (CH), 127.4 (C), 126.2 (CH), 121.4 (CH), 115.6 (CH), 110.3 (CH), 77.7 (C), 55.9 (CH₃). IR (KBr disc, cm⁻¹) 3512, 2962, 2936, 2838, 1582, 1527, 1491, 1439, 1270, 1243, 1212, 1172, 1135, 1123. MS (70 eV) m/z (%): 332 (M⁺, 100), 317 (M⁺—CH₃, 58), 289 (34). HRMS calcd for C₁₁H₉O₂SI 331.9368. Found 331.9369.

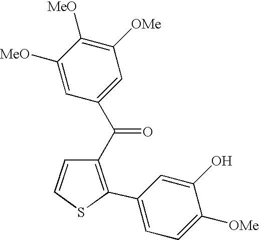

### 2-(3′-Acetoxy-4′-methoxyphenyl)-3-(3″,4″,5″,-trimethoxybenzoyl)thiophene (BLF-89-3)

t-Butyllithium (0.738 mL, 1.7 M in hexanes, 1.25 mmol) was added to a solution 3-iodo-2-(3′-acetoxy-4′-methoxyphenyl)thiophene (139 mg, 0.418 mmol) in dry THF (4 mL) at −78°° C. (dry-ice/acetone bath). To this was added a solution of 3,4,5-trimethoxybenzoyl chloride (108 mg, 0.47 mmol) in dry THF (1.5 mL) and the reaction mixture warmed to room temperature. The mixture was diluted with diethyl ether (50 mL) and washed with NH₄Cl_{(aq)} (sat., 50 mL), NaHCO₃(10 (5%, 60 mL) dried over MgSO₄ and concentrated onto silica gel (2 g). The residue was subject to flash chromatography (silca gel, hexane/diethyl ether 4:1, 2:1, 1:1) and the product obtained as a white solid (109 mg, 65%) mp=151-2° C. ¹H NMR (300 MHz, CDCl₃) δ 7.64 (d, J=5.0 Hz, 1H), 7.29-7.24 (m, 2H), 7.22 (dd. J=2.1, 8.4 Hz, 1H), 7.15 (s, 2H), 6.89 (d, J=8.4 Hz, 1H), 5.79 (br s, 1H), 3.94 (s, 6H), 3.92 (s, 6H). ¹³C NMR+APT (75.5 MHz, CDCl₃) δ 187.0 (C), 153.3 (C), 153.0 (C), 147.6 (C), 146.0 (C), 141.6 (C), 141.2 (C), 135.8 (CH), 133.4 (C), 126.8 (C), 123.1 (CH), 118.6 (CH), 112.5 (CH), 111.0 (CH), 106.7 (CH), 61.1 (CH₃), (56.4(CH₃), 56.1(CH₃). IR (KBr disc, cm⁻¹) 3212, 2993, 2939, 2839, 1574, 1453, 1428, 1348, 1259, 1240, 1126. MS (70 eV) m/z (%): 400 (M⁺, 100), 385 (M⁺—CH₃, 12). ERMS calcd for C₁₁H₂₀O₆S 400.0981. Found 400.0982.

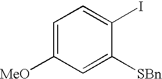

### Benzyl 2-iodo-5-methoxyphenyl sulfide

HBF₄ (50% w/v in H₂O, 14 mL) was added to a stirred suspension of 2-iodo-5-methoxyaniline¹⁴ (5.00 g, 21.5 mmol) in H₂O (30 ml) and the suspension stirred at room temperature for 0.5 h. The resultant clear solution was cooled in an ice bath, giving a white suspension. To this suspension NaNO₂ (1.55 g, 22.5 mmol) in H₂O (10 mL) was added dropwise over 0.1 h and the reaction mixture warmed to room temperature. The resulting suspension was filtered, rinsed with water (50 mL) and diethyl ether (25 mL) and dried under vacuum to give the corresponding diazonium tetrafluoroborate as a cream-colored solid 7.00 g (94%).

The diazonium salt (7.00 g, 20.1 mmol) obtained above was added portionwise to a solution of potassium ethyl xanthate (3.42 g, 21.0 mmol) in acetone (50 mL) at 0° C. (ice bath) over 0.15 h. The reaction mixture was stirred at 0° C. for 0.75 h and at room temperature for 1.0 h. This mixture was concentrated under reduced pressure diluted with diethyl ether (50 mL) and washed sequentially with H₂O (50 mL), KOH (2% w/v in H₂O, 50 mL), brine (50 mL). The organic layer was died over MgSO₄ and concentrated under reduced pressure. The residue was dissolved in methanol (50 mL) and powdered KOH (3.38 g, 60 mmol) added and the reaction mixture stirred vigorously for 3 h. The methanol was then evaporated under reduced pressure. The residue was suspended in H₂O (40 mL) and CH₂Cl₂ (40 mL). Benzyl chloride (2.43 mL, 34.0 mmol) and n-Bu₄NHSO₄ (100 mg) were added and the biphasic mix stirred vigorously for 1 h. The CH₂Cl₂ layer separated and the aqueous layer extracted with CH₂Cl₂ (50 mL). The combined CH₂Cl₂ fractions dried over MgSO₄ and concentrated on to silica gel (8 g). The solid residue was subjected to flash chromatography (silica gel, hexane/diethyl ether 98:2) and the product was obtained as a colorless oil which crystallized upon standing at 4° C. to afford a cream solid (4.22 g, 59%), (55% from 2-iodo-5-methoxyaniline) mp 72-4° C. ¹H NMR (300 MHz, CDCl₃) δ 7.69 (d, J=8.7 Hz, 1H), 7.29-7.46 (m, 5H), 6.82 (d, J=3.0 Hz, 1H), 6.48 (dd, J=3.0, S.7 Hz, 1H), 4.16 (s, 2H), 3.70 (s, 3H). ¹³C NMR+APT (75.5 MHz, CDCl₃) δ 159.7 (C), 142.1 (C), 139.4 (CH), 135.5 (C), 128.7 (CH), 128.3 (CH), 127.1 (CH), 113.7 (CH), 112.6 (CH), 87.4 (C), 55.0 (CH₃), 38.5 (CH₂). IR (KBr disc, cm⁻¹) 2955, 2930, 1558, 1494, 1426, 1283, 1228, 1038. MS (70 eV) m/z (%): 356 (M⁺, 45), 229 (10), 196 (22), 181 (6), 138 (15), 123 (20), 91 (100). HRMS calcd for C₁₄H₁₃OSI 355.9732. Found 355.9728

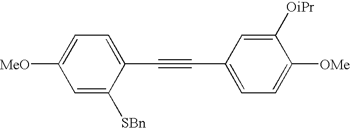

### Benzyl 2-[2′-(3″-isopropoxy-4″-methoxyphenyl)-ethynyl]-5-methoxyphenyl sulfide

n-Butyllithium (2.5 mL, 2.5 M in hexanes, 6.25 mmol) was added dropwise to a solution of β,β-dibromo-3-isopropoxy-4-methoxystyrene¹³ (1.09 g, 3.12 mmol) in THF (10 mL) at −78° C. (dry-ice/acetone). After the addition was complete the cold bath was removed and the reaction mixture allowed to warm to room temperature over 0.33 h. Dry zinc chloride (426 mg, 3.12 mmol) was then added and after it dissolved (approximately 3 min), Pd(PPh₃)₂Cl₂ (35.0 mg, 0.05 mmol) and 2-iodo-5-methoxyphenyl sulfide (890 mg, 2.50 mmol) were added. The resultant solution was stirred at room temperature for 1 h then diluted with diethyl ether (30 mL) washed with NH₄Cl_{(aq)} (saturated solution in H₂O, 30 mL) and brine (30 mL) dried over MgSO₄, and concentrated onto silica gel (3 g). The solid residue was subjected to flash chromatography (silica gel, hexane/diethyl ether 9:1 then 3:1) to give the product (R_{f}=0.25, 3:1) as a white solid (1.00 g, 96%) mp=67-8° C. ¹H NMR (300 MHz, CDCl₃) δ 7.43 (d, J=8.4 Hz, 1H), 7.40-7.24 (m, 5H), 7.15 (dd, J=1.8, 8.4 Hz, 1H), 7.09 (d, J=1.8 Hz, 1H), 6.84 (d, J=8.4 Hz, 1H), 6.79 (d, J=2.4 Hz, 1H), 6.68 (dd, J 2.4, 8.4 Hz, 1H), 4.55 (septet, J=6.0 Hz, 1H), 4.23 (s, 2H), 3.88 (s, 3H), 3.75 (s, 3H), 1.38 (d, J=6.0 Hz, 6H). ¹³C NMR+APT (75.5 MHz, CDCl₃) δ 159.5 (C), 150.8 (C), 146.9 (C), 141.1 (C), 136.8 (C), 133.6 (CH), 129.0 (CH), 128.6 (CH), 127.3 (CH), 125.1 (CH), 118.4 (CH), 115.6 (C), 115.4 (C), 113.3 (CH), 111.7 (CH), 111.2 (CH), 94.3 (C), 85.6 (C), 71.5 (CH), 56.0 (CH₃), 55.4 (CH₃), 37.5 (CH₂), 22.1 (CH₃). IR (KBr disc, cm⁻¹)2973, 2835, 1594, 1509, 1471, 1410, 1324, 1288, 1263, 1246, 1136, 1116, 1053. MS (70 eV) m/z (%): 418 (M⁺, 100), 376 (M⁺—CH₂═CHCH₃, 34), 341 (43), 299 (69), 253 (58) 91 (80). HRMS calcd for C₂₆H₂₆O₃S 418.1603. Found 418.1601.

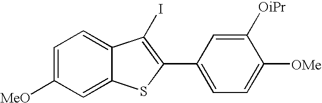

### 2-(3′-Isopropoxy-4′-methoxyphenyl)-3-iodo-6-methoxybenzo[b]thiophene

Iodine (556 mg, 2.19 mmol) was added to a solution of benzyl 2-[2′-(3″-isopropoxy-4″-methoxyphenyl)-ethynyl]-5-methoxyphenyl suifide (900 mg, 2.15 mmol) in CH₂Cl₂ (25 mL) and the solution stirred at room temperature for 1 h. After this time the solution was washed with Na₂S₂O₅ (5% w/v, 30 mL), dried over MgSO₄ and concentrated onto silica gel (5 g). The solid residue loaded onto a short column of (5 cm×2 cm) and eluted with hexane and hexane/diethyl ether 3:1 to give the product (R_{f}=0.33, 3:1)) as a white solid (950 mg, 97%), mp=102-3° C. ¹H NMR (300 MHz, CDCl₃) δ 7.69 (d, J=8.7 Hz, 1H), 7.30 (d, J=2.1 Hz, 1H), 7.26 (d, J=2.4 Hz, 1H), 7.21 (dd, J=2.1 Hz, 8.4 Hz, 1H), 7.07 (dd, J=2.4, 8.7 Hz, 1H), 6.94 (d, J=8.4 Hz, 1H), 4.63 (septet, J=6.3 Hz, 1H), 3.92 (s, 3H), 3.88 (s, 3H), 1.45 (d, J=6.3 Hz, 6H). ¹³C NMR+APT (75.5 MHz, CDCl₃) δ 158.0 (C), 150.5 (C), 146.7 (C), 139.5 (C), 139.3 (C), 135.9 (C), 126.9 (C), 126.5 (CH), 122.6 (CH), 116.7 (CH), 115.1 (CH), 111.4 (CH), 104.3 (CM), 77.7 (C), 71.3 (CH), 55.9 (CH₃), 55.6 (CH₃), 22.1 (CH₃). IR (KBr disc, cm⁻¹) 2974, 2920, 2835, 1600, 1530, 1493, 1471, 1261, 1224, 1138, 1020. MS (70 eV) m/z (%): 454 (M⁺, 66), 412 (M⁺—CH₂═CHCH₃, 58), 397 (26), 279 (24), 149 (100). Calcd for C₁₉H₁₉O₃SI C: 50.23; H, 4.22. Found C, 50.27; H, 4.19.

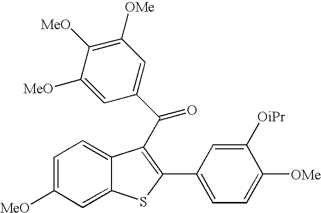

### 2-(3′-Isopropoxy-4′-methoxyphenyl)-6-methoxy-3-(3″,4″,5″-trimethoxybenzoyl)benzo[b]thiophene

t-Butyllithium (0.52 mL, 1.7 M in hexanes, 0.88 mmol) was added to a solution 3-iodo-2-(3′-isopropoxy-4′-methoxyphenyl)-6-methoxybenzo[b]thiophene (200 mg, 0.44 mmol) in dry THF (4 mL) at −78° C. (dry-ice/acetone bath). To this was added a solution of 3,4,5-trimethoxybenzoyl chloride (108 mg, 0.47 mmol) in dry THF (1.5 mL) and the reaction mixture warmed to room temperature. The mixture was diluted with diethyl ether (50 mL) and washed with NH₄Cl_{(aq)} (sat., 50 mL), NaHCO₃(aq) (5%, 60 mL) dried over MgSO₄ and concentrated onto silica gel (2 g). The residue was subject to flash chromatography (silca gel, hexane/diethyl ether 4:1, 2:1, 1:1) and the product obtained as a colorless resin (200 mg, 87%). ¹H NMR (300 MHz, CDCl₃) δ 7.65 (d, J=9.0 Hz, 1H), 7.32 (d, J=2.1 Hz, 1H), 7.10 (s, 2H), 7.00 (m, 2H), 6.85 (d, J=2.1 Hz, 1H), 6.75 (d, J=8.4 Hz, 1H), 4.30 (septet, J=6.0 Hz, 1H), 3.88 (s, 3H), 3.83 (s, 3H), 3.79 (s, 3H), 3.72 (s, 6H), 1.23 (d, J=6.0 Hz, 6H). ¹³C NMR+APT (75.5 MHz, CDCl₃) δ 192.9 (C), 157.7 (C), 152.7 (C), 150.8 (C), 147.0 (C), 143.4 (C), 142.6 (C), 140.0 (C), 133.8 (C), 132.1 (C), 129.7 (C), 126.1 (C), 124.0 (CH), 121.8 (CH), 116.6 (CH), 114.9 (CH), 111.6 (CH), 107.3 (CH), 104.3 (CH), 71.5 (CH), 60.8 (CH₃), 56.0 (CH₃), 55.8 (CH₃), 55.5 (CH₃), 21.8 (CH₃). IR (NaCl film, cm⁻¹) 2936, 1644, 1581, 1531, 1501, 1473, 1413, 1228, 1126. MS (70 eV) m/z (%): 522 (M⁺, 100), 480 (M⁺—CH₂═CHCH₃, 58), 301 (7), 195 (18). HRMS calcd for C₂₉H₃₀O₇S 522.1712. Found 522.1716

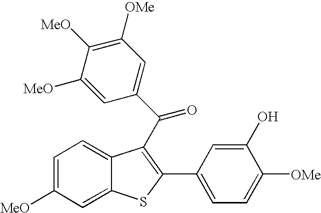

### 2-(3′-Hydroxy-4′-methoxyphenyl)₆-methoxy-3-(3″,4″,5″-trimethoxybenzoyl)benzo[b]thiophene (BLF-861)

Aluminium trichloride (86 mg, 0.64=mol) was added to a solution of 2-(3′-Isopropoxy-4′-methoxyphenyl)-6-methoxy-3-(3″,4″0.5″-trimethoxybenzoyl)benzo[b]thiophene (140 mg, 0.27 mmol) in dry dichloromethane (4 mL) and the solution stirred at room temperature for 1.5 h. After this time NH₄Cl_{(aq)} (sat. 20 mL) was added and the mixture extracted with diethyl ether (20 mL) dried over MgSO₄ and concentrated onto silica gel (1 g). The residue was subject to flash chromatography (silica gel, hexane/dichloromethane I diethyl ether 3:3:1) giving the product as a white solid (112 mg, 87%), mp=123-5° C. ¹H NMR (300 MHz, CDCl₃) δ 7.67 (d, J=9.0 Hz, 1H), 7.31 (d, J=2.4 Hz, 1H), 7.06 (s, 2H), 7.00 (dd, J=2.4, 9.0 Hz, 1H), 6.98 (d, J=2.1 Hz, 1H), 6.83 (dd, J=2.1, 9.0 Hz, 1H), 6.64 (d, J=9.0 Hz, 1H), 5.68 (s, 1H), 3.88 (s, 3H), 3.83 (s, 3H), 3.79 (s, 3H), 3.73 (s, 6H). ¹³C NMR+APT (75.5 MHz, CDCl₃) δ 192.9 (C), 157.7 (C), 152.6 (C), 146.9 (C), 146.4 (C), 143.7 (C), 142.3 (C), 140.0 (C), 133.7 (C), 132.4 (C), 129.9 (C), 126.7 (C), 124.2 (CH), 121.3 (CH), 115.1 (CH), 114.9 (CH), 110.4 (CH), 107.3 (CH), 60.8 (CH₃), 56.0 (CH₃), 55.8 (CH₃), 55.5 (CH₃). IR (KBr disc, cm⁻¹) 3402, 2934, 1649, 1580, 1499, 1474, 1413, 1324, 1266, 1228, 1158, 1125. MS (70 eV) m/z (%): 480 (M⁺, 100), 301 (6), 195 (7). HRMS calcd for C₂₆H₂₄O₇S 480.1243. Found 480.1242.

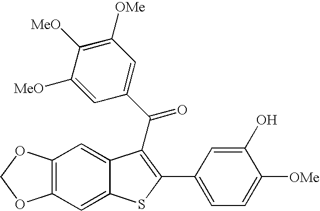

### 2-(3′-Hydroxy-4′-methoxyphenyl)-5,6-methylendioxy-3-(3″,4″,5″-trimethoxybenzoyl)benzo[b]thiophene (BLF-53-3)

Compound BLF-53-3 was prepared using a similar of reaction sequence as described for BLF-86-1 giving the product as a white solid, mp=156-7° C.: ¹H NMR (300 MHz, CDCl₃) δ 7.24 (s, 1H), 7.21 (s, 1H), 7.05 (s, 2H), 6.95 (d, J=2.1 Hz, 1H), 6.80 (dd, J=2.1, 8.4 Hz, 1H), 6.64 (d, J=8.4 Hz, 1H), 6.03 (s, 2H), 5.56 (s, 1H), 3.82 (s, 3H), 3.82 (s, 3H), 3.75 (s, 6H), ¹³C NMR+APT (75.5 MHz, CDCl₃) δ 192.9 (C), 152.7 (C), 147.3 (C), 147.0 (C), 146.9 (C), 145.5 (C), 144.6 (C), 142.4 (C), 134.4 (C), 132.5 (2×C), 130.1 (C), 126.9 (C), 121.3 (CH), 115.1 (CH), 110.5 (CH), 107.4 (CH), 102.5 (CH), 101.5 (CH₂), 101.1 (C), 60.9 (CH₃), 56.1 (2×CH₃), 55.9 (CH₃). IR (KBr disc, cm⁻¹) 3418, 2939, 1628, 1582, 1501, 1465, 1335, 1279, 1232, 1124. MS (70 eV) m/z (%): 494 (Mt, 100), 480(3), 301 (10)267 (8). HRMS calcd for C₂₆H₂₂O₈S 494.1035. Found 494.1038. 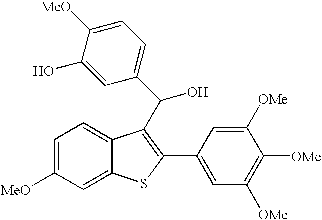
3-(α-Hydroxy-3′-hydroxy-4′-methoxybenzyl)-6-methoxy-3-(3″,4″,5″-trimethoxybenzoyl)benzo[b]thiophene (BLF-34-3):

Compound BLF-34-3 was prepared using a similar of reaction sequence as described for BLF-86-1 except that O-acylisovanillin was used as the electrophile (9) and the product deacetylated in situ by addition of methanol and K₂CO₃, giving the product as a white solid: ¹H NMR (300 MHz, CDCl₃) δ 7.71 (d, J=8.4 Hz, 1H), 9.29 (d, J=2.1 Hz, 1H), 7.00 (d, J=2.1 Hz, 1H), 6.82 (m, 2H), 6.80 (d, J=8.4 Hz, 1H), 6.69 (s, 2H) 6.20 (br s, 1H), 5.62 (br s, 1H), 3.88 (s, 3H), 3.82 (s, 6H), 3.78 (s, 6H).

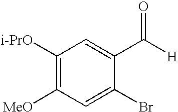

### 2-Bromo-5-isopropoxy-4-methoxybenzaldehyde

NBS (1.19 g, 6.70 mmol) was added to a solution of isopropyl isovanillin ether (1.24 g, 6.39 mmol), in DMF (6 mL) at room temperature, and heated to 80° C. The reaction was monitored by TLC (eluent, hexane/diethylether 1:1, product R=0.63) and after 7 h was cooled to room temperature. The solution was diluted with diethylether (100 mL), washed with Na₂S₂O₅ (5% w/v, 100 mL), water (2×100 mL), dried over MgSO₄, and concentrated under reduced pressure onto silica gel (5 g). The solid residue was subjected to flash chromatography (silica gel, hexane/diethylether 95:5, then 9:1) to give the product (R_{f}=0.09, 95:5) as a white solid (1.65 g, 94%), mp=78-79° C. ¹H-NMR (CDCl₃) δ 10.16 (s, 1H, —CHO), 7.41 (s, 1H, Ar—H), 7.04 (s, 1H, Ar—H), 4.61 (septet, J=6.0, 1H, —OCH(CH₃)₂), 3.92 (s, 3H, —OCH₃), 1.36 (d, J=6.0 Hz, 6H, —OCH(CH₃)₂). ¹³C-NMR (CDCl₃) δ 190.8 (C═O), 155.6, 147.1 (C—O), 126.4 (C—CHO), 120.0 (C—Br), 115.8, 113.5 (C—H), 71.5 (—CH(CH₃)₂), 56.4 (—OCH₃), 21.8 (2×—CH(CH₃)₂). LRMS (Calculated for C₁₁H₁₃₀₃⁸¹ Br): m/z=274 (21) (M⁺), 232 (100) (M⁺—CH₂═CH—CH₃). HRMS Calculated for C₁₁H₁₃O₃⁸¹Br=274.0027. Found=274.0024. IR (KBr disc, cm⁻¹) v max=2971, 1681, 1587, 1508, 1432, 1386, 1268, 1217, 1158, 1109.

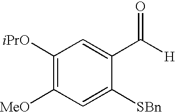

### Benzyl 2-formyl-4-isopropoxy-5-methoxyphenyl sulfide

Benzylmercaptan (1.20 mL, 10.2 mmol) was added to a stirring suspension of NaH (490 mg, 10.2 mmol) in DMF at 0° C. (ice-bath). The reaction was left to stir at 0° C. until hydrogen evolution had ceased. To this was added the bromobenzaldehyde above (2.78 g, 10.2 mmol), and the reaction was left to stir for 0.25 h then warmed to room temperature. After 24 h, the reaction was diluted with diethylether (100 mL), washed with HCl (40 mL, 1 M), NaOCl (1%, 40 mL) and water (3×100 mL), dried over MgSO₄, and concentrated under reduced pressure onto silica gel (8 g). The solid residue was subjected to flash chromatography (silica gel, hexane/diethylether 1:4), to give the product (R_{f}=0.16) as a yellow oil (2.87 g, 89%). ¹H-NMR (CDCl₃) δ 10.21 (s, 1H, Ar—H), 7.35 (s, 1H, Ar—H), 7.22-7.09 (m, 5H, Ar—H), 6.77 (s, 1H, Ar—H), 4.63 (septet, J=5.7 Hz, 1H, —OCH(CH₃)₂), 3.98 (s, 2H, —CH₂—), 3.77 (s, 3H, —OCH₃), 1.36 (d, J=5.7 Hz, —OCH(CH₃)₂). ¹³C-NMR (CDCl₃) δ 190.6 (C═O), 154.5, 147.4 (C—O), 137.0, 132.2, 130.4 (C), 128.9, 128.4 (2×C—H), 127.3, 117.1, 113.1 (C—H), 71.2 (—CH(CH₃)₂), 56.1 (—OCH₃), 41.7 (CH₂), 21.8 (CH(CH₃)₂). LRMS m/z=316 (26) (M⁺), 274 (11) (M⁺—CH₂═CH—CH₃), 225 (17) (M⁺—CH₂(C₆H₅)), 183 (59) (M⁺—CH₂═CH—CH₃, —CH₂(C₆H₅)) HRMS Calculated for C₁₈H₂₀O₃S=316.1133. Found=316.1144. IR (NaCl film, cm⁻¹) v max=2988, 2861, 1666, 1578, 1495, 1439, 1388, 1349, 1334, 1262, 1158.

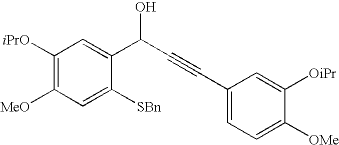

### 1-(2′-Benzylthio-5′-isopropoxy-4′-methoxyphenyl)-3-(3″-isopropoxy-4″-methoxyphenyl)prop-2-yn-1-ol

n-Butyllithium (0.63 mL, 1.27 mmol) was added dropwise to a solution of 8,Adibromo-3-isopropoxy-4-methoxystyrene¹³ (221 mg, 0.633 mmol) in THF (1.5 mL) at −78° C. (dry-ice/acetone). The solution was warmed to room temperature, then recooled to −78° C. and aldehyde above (190 mg, 0.601 mmol), added and left to stir for 0.25 h. The reaction was bought to room temperature again, quenched with NH₄Cl_{(aq)} (10%, 40 mL), taken up into diethylether (40 mL), washed with water (30 mL), dried over MgSO₄, and concentrated under reduced pressure onto silica gel (2 g). The solid residue was subjected to flash chromatogaphy (silica gel, hexane/diethylether 3:2) to give the product (R_{f}=0.16) as a yellow resin (236.1 mg, 74%): ¹H-NMR (CDCl₃) δ 7.30 (s, 1H, Ar—H), 7.25-7.09 (m, 5H, Ar—H), 7.01 (dd, J=1.8, 8.3 Hz, 1H, 6″-H), 6.95 (d, J=1.8 Hz, 1H, 2″-H), 6.77 (d, J=8.3 Hz, 1H, 5″-H), 6.75 (s, 1H, Ar—H), 6.07 (d, J=5.1 Hz, 1H, 1-OH), 4.62 (septet, J=6.1 Hz, 1H, —OCH(CH₃)₂), 4.45 (septet, J=6.1 Hz, 1H, —OCH(CH₃)₂), 3.99 (s, 2H, —CH₂), 3.83 (s, 3H, —OCH₃), 3.70 (s, 3H, —OCH₃), 2.40 (d, J=5.1 Hz, 1H, 1-H), 1.38 (dd, J=3.7, 6.1 Hz, 6H, —OCH(CH₃)₂), 1.33 (d, J=6.1 Hz, 6H, —OCH(CH₃)₂). ¹³C-NMR (CDCl₃) δ 150.9, 149.7, 148.0, 146.8 (C—O), 138.2, 137.5 (C), 129.1, 128.5 (2×C—H), 127.2, 125.2 (C—H), 123.0 (C), 119.0, 118.6 (C—H) 114.8 (C), 114.2, 111.6 (C—H), 87.9, 86.4 (—C≡C—), 71.4, 71.3 (CH(CH₃)₂), 63.0 (1-C), 56.0, 55.9 (—OCH₃), 41.8 (—CH₂—), 22.2, 22.0, 21.8 (2×CH(CH₃)₂). LRMS m/z=506 (43) (M⁺), 415 (100) (M⁺—CH₂C₆H₅). HRMS Calculated for C₃₀H₃₄₀₅S=506.2126. Found=506.2131. IR (NaCl film, cm⁻¹) v max=2978, 1592, 1504, 1386, 1264, 1110, 1044.

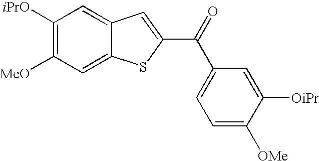

### 5-Isopropoxy-2-(3′-isopropoxy-41-methoxybenzoyl)-6-methoxybenzo[b]thiophene

Iodine (418 mg, 1.65 mmol) was added to a solution of the alcohol above (835 mg, 1.65 mmol) in dichloromethane (5 mL) at room temperature. The reaction was monitored by TLC (eluent hexane/diethylether/dichloromethane 4:3:3, product R_{f}=0.53), and after 3 h the dichloromethane was removed under reduced pressure. The resultant red oil was taken up into diethylether (100 mL), and washed with Na₂S₂O₅ (10%, 100 μL), water (100 mL), dried over MgSO₄, and concentrated under reduced pressure onto silica gel (5 g). The solid residue was subjected to flash chromatography (silica gel, hexane/diethylether 5:3) to give the product (R_{f}=0.14) as a yellow oil (591 mg, 86%). ¹H—NMR (CDCl₃) δ 7.74 (s, 1H, Ar—H), 7.58 (dd, J=2.0, 8.4 Hz, 1H, 6′-H), 7.49 (d, J=2.0 Hz, 1H, 2′-H), 7.29 (s, 1H, Ar—H), 7.28 (s, 1H, Ar—H), 6.95 (d, J=8.4 Hz, 1H, 5′-H), 4.60 (septet, J=6.0 Hz, 2H, 2×—OCH(CH₃)₂), 3.96 (s, 3H, —OCH₃), 3.94 (s, 3H, —OCH₃), 1.41 (d, J=2.4 Hz, 6H, —OCH(CH₃)₂), 1.39 (d, J=2.6 Hz, 6H, —OCH(CH₃)₂). ¹³C-NMR (CDCl₃) δ 187.7 (C═O), 153.9, 152.0, 147.0, 146.7, 141.2, 136.9, 132.6 (C), 131.3 (C—H), 130.6 (C), 123.8, 115.7, 110.5, 110.2, 103.8 (C—H), 71.6, 71.4 (CH(CH₃)₂), 56.1, 56.0 (—OCH₃), 21.9, 21.8 (2×CH(CH₃)₂). LRMS m/z=414 (59) (M⁺), 330 (100), (M⁺−2×CH₂═CH—CH₃). HRMS Calculated for C₂₃H₂₆₀₅S=414.1500. Found=414.1493. IR (NaCl film, cm⁻¹) v max=2975, 1624, 1594, 1500, 1463, 1291, 1268, 1235, 1211, 1136.

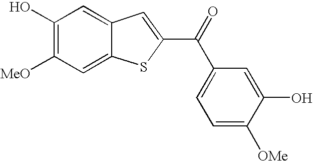

### 5-Hydroxy-2-(3′-hydroxy-4′-methoxybenzoyl)-6-methoxybenzo[b]thiophene (KH-2-2)

5-Isopropoxy-2-(3′-isopropoxy-4′-methoxybenzoyl)-6-methoxybenzo[b]thiophene (above) was reacted with 5 equivalents of AlCl₃ as described for BLF-86-1, giving the product, KH-2-2, in a 91% yield:

¹H-NMR (CDCl₃) δ 7.73 (s, 1H), 7.50 (d, J=2.1 Hz. 1H), 7.47 (dd, J=2.1, 8.3 Hz), 7.29 (s, 1H), 7.24 (s, 1H), 6.92 (d, J=8.3 Hz), 5.78 (s, 1H), 5.75 (s, 1H), 3.97 (s, 3H), 3.95 (s, 3H).

¹³C-NMR (CDCl₃) δ 187.9, 150.1, 148.5, 145.3, 144.9, 141.6, 135.2, 133.2 (C), 131.5 (C—H), 131.3 (C), 122.7, 115.5, 109.9, 109.1, 102.9 (C—H), 56.2, 55.1 (CH₃).

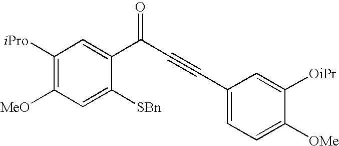

### 1-(2′-Benzylthio-5′-isopropoxy-4′-methoxyphenyl)-3-(3″-isopropoxy-4″-methoxyphenyl)prop-2-yn-1-one

Prepared by DDQ oxidation of 1-(2′-benzylthio-5′-isopropoxy-4′-methoxyphenyl)-3-(3″-isopropoxy-4″-methoxyphenyl)prop-2-yn-1-ol (1.2 equivalents of DDQ in CH₂Cl₂). OR By reaction of benzyl (2-bromo-4-isopropoxy-5-methoxyphenyl)sulfide with one equivalent of nBuLi at −78° C. in THF followed by addition of 3-(3′-isopropoxy-4′-methoxyphenyl)propynoyl chloride (1.1 equivalents)

¹H-NMR (CDCl₃) δ 7.91 (s, 1H), 7.45-7.21 (m, 6H), 7.13 (d, J=1.6 Hz, 1H), 6.86 (d, J=8.3 Hz, 1H), 6.76 (s, 1H), 4.55 (septet, J=6.1 Hz, 1H), 4.52 (septet, J=6.1 Hz, 1H), 4.17 (s, 2H), 3.87 (s, 3H), 3.75 (s, 3H), 1.40 (d, J=6.1 Hz, 6H), 1.37 (d, J=6.1 Hz, 6H). ¹³C-NMR (CDCl₃) δ 176.0, 154.3, 152.6, 146.9, 143.4, 136.9, 136.4 (C), 128.7, 128.5, 127.2, 126.9, (C—H), 126.7 (C), 120.9, 119.0 (C—H), 112.0 (C), 111.5, 109.2, (C—H), 93.4, 86.8 (C), 71.9, 71.4 (C—H), 55.9, 55.8 (CH₃), 37.3 (CH₂), 21.9, 21.8 (CH₃).

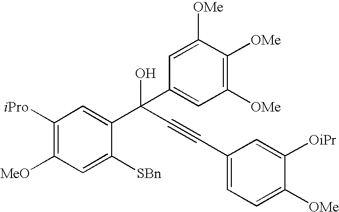

### 1-(2′-benzylthio-5′-isopropoxy-4′-methoxyphenyl)-1-(3″,4″,5″-trimethoxyphenyl)-3-(3′″-isopropoxy-4′″-methoxyphenyl)prop-2-yn-1-ol

t-BuLi (0.57 mL, 0.938 mmol) was added dropwise to a solution of iodo-3,4,5-trimethoxybenzene (138 mg, 0.469 mmol) in THF (3 mL) at −78° C. (dry-ice/acetone bath), and left to stir for 0.5 h. After this time, 1-(2′-benzylthio-5′-isopropoxy-4′-methoxyphenyl)-3-(3″-isopropoxy-4″-methoxyphenyl)prop-2-yn-1-one (237 mg, 0.469 mmol) was dissolved in THF (2 mL) and added dropwise to the solution. After further stirring for 0.5 h, the solution was warmed to room temperature and quenched with NH₄Cl_{(aq)} (40 mL, 10%). The solution was extracted with diethyl ether (3×50 mL), washed with water (40 mL), dried over MgSO₄, and concentrated onto silica gel (2 g). The solid residue was subjected to flash chromatography (silica gel, hexanes: diethyl ether, 1:1, 2:3 sequential elution) to give the product (R_{f}=0.21, 3:2 diethyl ether: hexanes) as a yellow oil (277 mg, 88%).

¹H-NMR (CDCl₃) δ 7.23-7.20 (m, 3H), 7.14 (s, 11H), 7.10-7.05 (m, 3H), 6.99 (d, J=2 Hz, 11H), 6.90 (s, 2H), 6.78 (d, J=8.4 Hz), 6.59 (s, 1H), 5.38 (s, 11H), 4.42 (septet, J=6.1 Hz, 2H), 3.87 (s, 2H), 3.86 (s, 3H), 3.84 (s, 3H), 3.81 (s, 6H), 3.59 (s, 3H), 1.35 (d, J=6.1 Hz, 6H), 1.33 (d,J=6.1 Hz, 6H).

¹³C-NMR (CDCl₃) 152.8, 151.1, 148.8, 146.9, 146.4, 140.6, 139.7, 137.4, 137.3 (C), 129.3, 128.4, 127.2, 125.2 (C—H), 122.5 (C), 120.7, 118.6, 115.8 (C—H), 114.6 (C), 111.6, 104.3 (C—H), 90.1, 88.0, 75.4 (C), 71.4, 71.2 (C—H), 60.8, 56.9, 55.9, 55.8 (CH₃), 41.2 (CH₂), 21.9, 21.8 (CH₃).

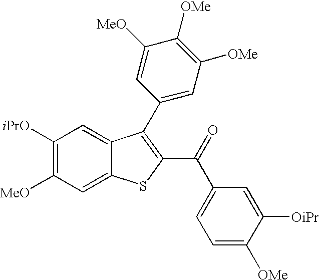

### 5-Isopropoxy-2-(3′-isopropoxy-4′-methoxybenzoyl)-6-methoxy-3-(3″,4″,5″-trimethoxyphenyl)benzo[b]thiophene

Iodine (80 mg, 0.317 mmol) was added to a solution of 1-(2′-benzylthio-5′-isopropoxy-4′-methoxyphenyl)-1-(3″,4″,5″-trimethoxyphenyl)-3-(3′″-isopropoxy-4′″-methoxyphenyl)prop-2-yn-1-ol (208 mg, 0.309 mmol) in CH₂Cl₂ (3 mL) and left to stir for 0.5 h. After this time, the solution was quenched with Na₂S₂O₅ (aq) (40 mL, 5%), and the solution extracted with diethyl ether (3×50 mL), washed with water (100 mL), dried over MgSO₄, and concentrated under reduced pressure onto silica gel (2 g). The solid residue was subjected to flash chromatography (silica gel, hexanes:diethyl ether, 2:3 eluent) to give the product (R_{f}=0.21) as a red solid (164 mg, 91%).

¹H-NMR (CDCl₃) δ 6.95 (dd, J=1.9, 8.4 Hz, 1H), 6.91 (d, J=1.9 Hz, 1H), 6.90 (s, 2H), 6.87 (d, J=8.4 Hz, 1H), 6.73 (s, 1H), 6.56 (s, 1H), 4.53 (septet, J=6.0 Hz, 1H), 4.38 (septet, J=6.0 Hz, 1H), 3.89 (s, 6H), 3.85 (s, 3H), 3.82 (s, 3H), 3.78 (s, 3H), 1.40 (d, J=6.0 Hz, 6H), 1.32 (d, J=6.0 Hz, 6H)

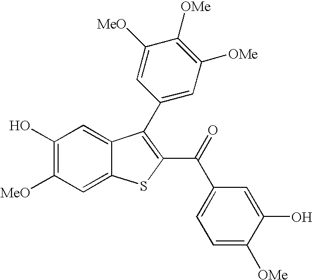

### 5-Hydroxy-2-(3′-hydroxy-4′-methoxybenzoyl)-6-methoxy-3-(3″,4″,5″-trimethoxyphenyl)benzo[b]thiophene

Aluminium trichloride (17 mg, 0.138 mmol) was added to a solution of 5-Isopropoxy-2-(3-isopropoxy-4′-methoxybenzoyl)-6-methoxy-3-(3′,4″,5″-trimethoxyphenyl)benzo[b]thiophene (27 mg, 0.0466 mmol) in CH₂Cl₂ (3 mL) at room temperature and left to stir. After 1 h, a further amount of aluminium trichloride (17 mg, 0.138 mmol) was added. After a further 2 h, the solution was quenched with NH₄Cl_{(aq)} (30 mL, 10%), extracted with ethyl acetate (3×50 mL), dried over MgSO₄, and concentrated under reduced pressure onto silica gel (2 g). The solid residue was subjected to flash chromatography (silica gel, hexanes:diethyl ether, 4:1, 7:3, 3:2, 1:1 sequential elution) to give the product as a yellow solid (21 mg, 92%).

¹H-NMR (CDCl₃) δ 7.33 (s, 1H), 7.32 (s, 1H), 7.19 (dd, J=2.1, 8.3 Hz, 1H), 7.1 (d, J=2.1 Hz, 1H), 6.61 (d, J=8.3 Hz, 1H), 6.47 (s, 2H), 5.78 (s, 1H), 5.42 (s, 1H), 4.04 (s, 3H), 3.84 (s, 3H), 3.75 (s, 9H).

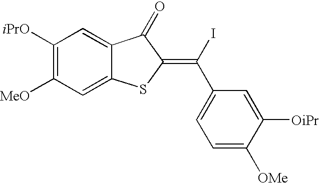

### 2-(α-iodo-3′-isopropoxy-4′-methoxybenzylidene)-5-isopropoxy-6-methoxybenzo[b]thiophen-3-one

Iodine (370 mg, 1.47 mmol) was added to a solution of 1-(2′-benzylthioxy-5′-isopropoxy-4′-methoxyphenyl)-3-(3″-isopropoxy-4″-methoxyphenyl)prop-2-yn-1-one (740 mg, 1.47 mmol) in CH₂Cl₂ (20 mL) and stirred for 0.5 h. After this time, the solution was quenched with Na₂S₂O_{5(aq)} (200 mL), extracted with diethyl ether (3×150 mL), dried over MgSO₄, and concentrated under reduced pressure onto silica gel (7 g). The solid residue was subjected to flash chromatography (silica gel, hexanes:diethyl ether, 3:2, 2:3 sequential elution), to give the product as a yellow oil (668 mg, 84%).

¹H-NMR (CDCl₃) δ 7.39 (s, 1H), 7.09 (dd, J=2.1, 8.4 Hz, 1H), 7.08 (s, 1H), 7.05 (d, J=2.1, 1H), 7.04 (dd, J=2.0, 8.4 Hz, 1H), 6.90 (d, J=8.4 Hz), 6.96 (d, J=2.0 Hz, 1H), 6.83 (d, J=8.4 Hz, 1H), 6.80 (s, 1H), 6.75 (s, 1H), 4.63-4.46 (m, 4H), 3.95 (s, 3H), 3.90 (s, 6H), 3.85 (s, 3H), 1.42-1.35 (m, 24H). ¹³C-NMR (CDCl₃) δ 178.9, 165.1, 157.2, 156.9, 151.5, 146.6, 146.3, 145.9, 139.3, 138.0, 137.7, 134.2, 133.6, 126.8, 124.9 (C), 122.1, 121.8, 116.2, 115.9 (C—H), 114.0 (C), 112.2, 111.4, 110.8, 110.5, 105.3, 103.8 (C—H), 98.7 (C), 71.6 (C—H), 56.5, 56.1 (CH₃), 21.9, 21.8 (CH₃).

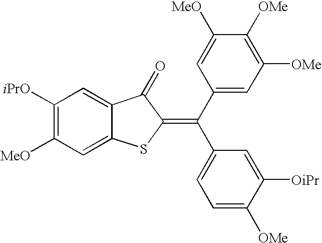

### 2-[α-(3″-isopropoxy-4″-methoxyphenyl)-3′,4′,5′-trimethoxybenzylidene]-5-isopropoxy-6-methoxybenzo[b]thiophen-3-one

t-Butyllithium (0.35 mL, 0.55 mmol) was added dropwise to a solution of iodo-3,4,5-trimethoxybenzene (80 mg, 0.27 mmol) in THF (3 mL) at −78° C. (dry-ice/acetone bath). After 0.1 h, dry zinc chloride (36 mg, 0.27 mmol) was added and the reaction warmed to room temperature. After 0.25 h, Pd(PPh₃)₂Cl₂ (4 mg, 3%) was added, followed by iodoaurone (68 mg, 0.13 mmol). The reaction was left to stir for 18 h, after which the solution was diluted with diethyl ether (40 mL), washed with water (30 mL), dried over MgSO₄, and concentrated under reduced pressure onto silica gel (2 g). The residue was subjected to flash chromatography (silica gel, hexanes/diethyl ether, 3:2, 2:3, 1:3 sequential elutions) to give the product as a white solid (52 mg, 69%). ¹H-NMR (CDCl₃) δ 7.30 (s, 1H), 7.28 (s, 1H), 6.97 (dd, J=2.0, 8.4 Hz, 1H), 6.81 (d, J=2.0 Hz, 1H), 6.73 (d, J=8.4 Hz, 1H), 4.55 (septet, J=6.1 Hz, 1H), 4.39 (septet, J=6.1 Hz, 1H), 3.95 (s, 3H), 3.82 (s, 3H), 3.79 (s, 3H), 3.1 (s, 6H), 1.38 (d, J=6.1 Hz, 6H), 1.24 (d, J=6.1 Hz, 6H).

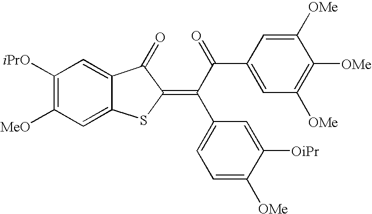

### 2-[2-Oxo-1-(3′-isopropoxy-4′-methoxyphenyl)-2-(3″,4″,5″-trimethoxyphenyl)-ethylidene]-5-isopropoxy-6-methoxybenzo[b]thiophen-3-one

t-Butyllithium (0.37 mL, 0.59 mmol) was added dropwise to a solution of iodo-3,4,5-trimethoxybenzene (87 mg, 0.29 mmol) in THF (5 mL) at −78° C. (dry-ice/acetone bath). After 0.1 h, dry zinc chloride, (40 mg, 0.29 mmol) was added and the reaction warmed to room temperature. After 0.25 h, Pd(PPh₃)₂Cl₂ (4 mg, 3%) was added, and the nitrogen atmosphere replaced with carbon monoxide. The solution was stirred vigourously, and after 0.2 h iodoaurone (68 mg, 0.13 mmol) was added. The reaction was left to stir for 19 h, after which time it was diluted with diethyl ether (50 mL), washed with water (30 mL), dried over MgSO₄, and concentrated under reduced pressure onto silica gel (2 g). The residue was subjected to flash chromatography (silica gel, hexanes/diethyl ether, 1:2, 1:3, 1:4 sequential elutions) to give the product as a white solid. ¹H-NMR (CDCl₃) δ 7.40 (s, 1H), 7.19 (dd, J=2.1, 8.3 Hz, 1H), 7.12 (d, J=2.1 Hz, 1H), 7.03 (s, 1H), 6.88 (s, 2H), 6.61 (d, J=8.3 Hz, 1H), 4.62 (septet, J=6.1 Hz, 1H), 4.58 (septet, J=6.1 Hz, 1H), 3.91 (s, 3H), 3.81 (s, 6H), 3.79 (s, 6H), 1.38 (d, J=6.1 Hz, 6H), 1.33 (d, J=6.1 Hz, 6H).

Biological Activity

A number of compounds according to the invention (FIG. 11) were examined for their effects on tubulin polymerization, colchicine binding and growth of Burkitt lymphoma CA46 cells according to the procedures described in Verdier-Pinard et al., Mol. Pharmacol., 1998, 53.62 and effects compared against Combretastatin A4(A) and benzothiophene(B) (see page 2). The results are depicted in Table 1.

TABEL 1
Effects of compounds on tubulin polymerisation, colchicine binding
and growth of Burkitt lymphoma CA46 cells (see FIG. 11)

| | | Inhibition | | |
|---|---|---|---|---|
| | | of colchicine | | Inhibition of |
| | Inhibition of tubulin | binding (% inhibition)^{b} | | CA46 cell |
| | Polymerization^{a} | 5 μM | 50 μM | growth |
| Compound | IC₅₀ (μM) | inhibitor | inhibitor | IC₅₀ (nM) |
| (A) | 2.1 ± 0.1 | 91 | ND | 1 (11^{e}) |
| (B)^{c} | >40^{*d} | — | 28 | 2000 (640^{e}) |
| BLF-86-1 | 3.5 ± 0.3 | 6 | 61 | 500 (520^{e}) |
| BLF-34-3 | 6.1 ± 0.8 | 5 | 73 | >1000 |
| BLF-53-3 | >40^{*d} | 2 | 31 | >1000 |
| GPF-60-1 | 3.6 ± 1 | 64 | 88 | 390^{e} |
| BLF-89-3 | 1.0 ± 0.1 | 67 | — | 300^{e} |
| BLF-28-1 | 0.75 | 55 | 80 | 40 (34^{e}) |
| BLF-68-3 | >40* | — | — | >1000^{e} |
| BLF-70-3 | 2.5 ± 0.3 | 17 | — | 400^{e} |
| BLF-62-3 | 1.3 ± 0.2 | 80 | — | 42^{e} |
| BLF-36-1 | >40^{*d} | — | — | >1000 |
| BLF-69-3 | 8.8 ± 1.4 | — | — | 560^{e} |
| BLF-61-3 | 4.1 ± 0.6 | 28 | — | 370^{e} |
| BLF-67-3 | 1.6 | 54 | — | 45^{e} |
| KH-2-2 | 2.4 ± 0.4 | — | — | — |
| DK-12a-1 | 10-40 | — | — | — |
| DK-2a-2 | 4-10 | — | — | — |

^{a}The tubulin concentration was 10 μM. Inhibition of extent of assembly was the parameter measured.
^{b}The tubulin concentration was 1.0 μM and the [³H]colchicine concentration was 5.0 μM.
^{c}Data from Pinney K.G. et at Bioorg. Med. Chem. Let, 1999, 1081 and U.S. Pat. 5,886,025.
^{d}The asterisk indicates that the rate but not the extent of assembly was inhibited by compound concentrations as high as 40 μM.
^{e}The MCF-7 human breast carcinoma cell line was used.

1. A combinatorial library of 2 or more chemical compounds each compound comprising the reaction product derived from at least two substances selected from (a), (b) and (c): 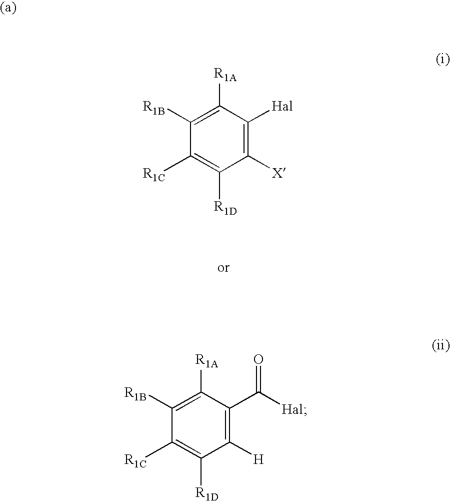 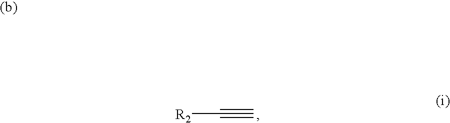
or a metallated form thereof; or 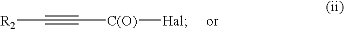
or 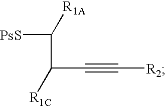
(c)
(i) R₃-L, or a metallated form thereof wherein L is replaced by a metal; or
(iii) R₃—C(O)-Hal;
wherein
R_{1A}-R_{1D} are independently selected from hydrogen, hydroxy, optionally substituted alkyl, optionally substituted alkoxy, optionally substituted acyloxy, amino, optionally substituted alkylamino, optionally substituted dialkylamino or any 2 adjacent R_{1A}-R_{1D} together form O—CH₂—O—:
Hal is I, Br or CI;
X′ is OH, SPs (wherein Ps is a sulfur-protecting group capable of stabilising a positive charge), NP_{N} (wherein P_{N} is a nitrogen-protecting group); or NHR (wherein R is sulphonyl, triflouroacyl, C₁₋₇acyl, C₁₋₆alkyl or an aryl group);
L is a leaving group,
R₂ and R₃ are optionally substituted aryl groups.

2. A combinatorial library of compounds for screening, as potential tubulin polymerisation inhibitors, said library comprising two or more compounds of formulae (E) to (Q), said compounds being the reaction products of the following substrates:
(a)(i), (b)(i) and (c)(i) to produce compounds of formulae (E) and (F) 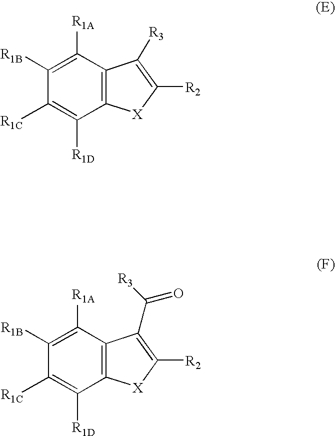
(a)(i), (b)(i) and (c)(ii) to produce compounds of formula (F);
(a)(i), (b)(ii) and (c)(i) to produce compounds of formulae (G), (H), (I), (J) or (K) 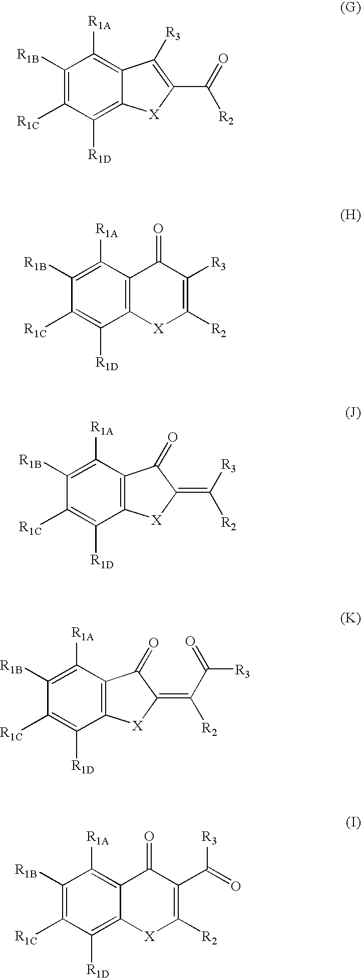
(a)(i), (b)(ii) and (c)(ii) to produce compounds of formulae (I) and (K)
(a)(ii), (b)(i) and (c)(i) to produce compounds of formula (L) 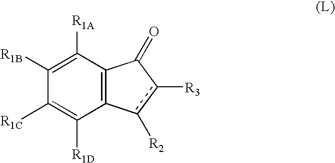
(a)(ii), (b)(i) and (c)(ii) to produce compounds of formula (M) 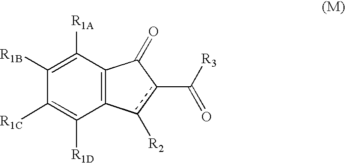
(b)(iii) and (c)(i) to produce compounds of formula (P) 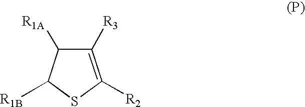
(b)(iii) and (c)(ii) to produce compounds of formula (Q) 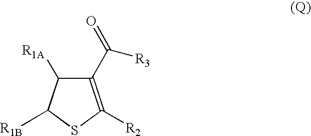
wherein R₂, R₃, R_{1A}-R_{1D}, (a)(i), (a)(ii), (b)(i)-(b)(iv) and (c)(i)-(iii) are as defined in claim 1, and X═O, S, or NR (wherein R is H, sulfonyl, C₁₋₆alkyl, C₁₋₇acyl or an aryl group).

3. A combinatorial library of compounds according to claim 1, in which the compounds are further attached to a solid support surface.

4. A combinational library of intermediates useful for the preparation of the compounds of formulae (E)-(Q) as defined in claim 2, said intermediates being the reaction products of the following substrates:
(a)(i) and (b)(i) to produce intermediates of formula (E′) for use in preparing compounds of formulae (E) and (F) 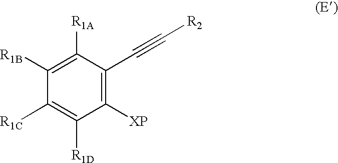
(a)(i) and (b)(ii) to produce intermediates of formulae (F′), (G′) and (H′) for use in preparing compounds of formulae (G), (H), (I), (J) or (K); 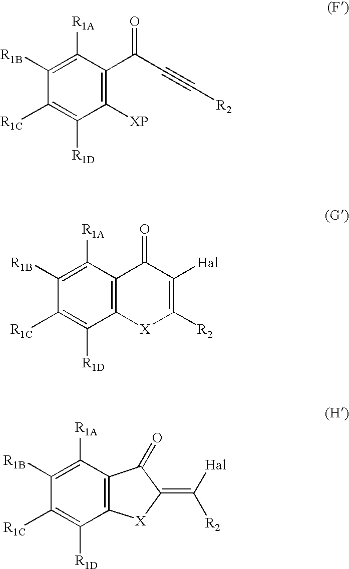
(a)(ii) and (b)(i) to produce intermediates of formulae (I′) and (J′) for use in preparing compounds of formula (L) or (M) 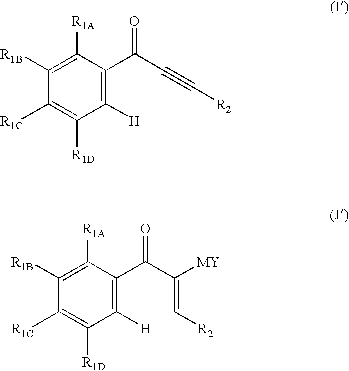
(b)(iii) with itself to produce intermediates of formulae (L′) 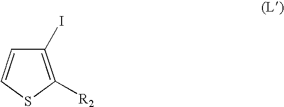
for use in preparing compounds of formulae (P) and (Q);
wherein R_{1A}-R_{1D}, R₂, R₃, X, (a)(i), (a)(ii), (b)(i)-(iv) and (c)(ii)-(iii) are as defined in claim 1, X is N, O or S, P is a protecting group and MY is Sn(alkyl)₃ or B(OR)₂, wherein R is H or alkyl.

5. A combinatorial library of intermediates according to claim 4, in which the intermediates are further attached to a solid support surface.

6. A combinatorial library of at least two compounds of formula (I): 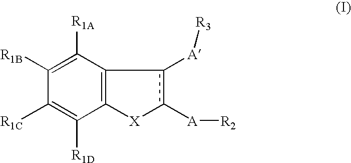
wherein
X is selected from O, S, NR, C═O(R is H, C₁₋₆alkyl or C₁₋₆acyl);
A and A′ are independently selected from CH₂, C═O, CH(OR′) (R′ is H, C₁₋₆alkyl, C₁₋₇acyl) or a single bond; 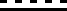
is a double or single bond
R_{1A}-R_{1D} are independently selected from hydrogen, hydroxy, optionally substituted alkoxy, optionally substituted alkyl, optionally substituted acyloxy, amino, optionally substituted alkylamino, optionally substituted dialkylamino, optionally substituted acylamino or any 2 adjacent R_{1A}-R_{1D} together form —O—CH₂—O—.
R₂ and R₃ are optionally substituted aryl groups.

7. A compound of formula (I′″″) 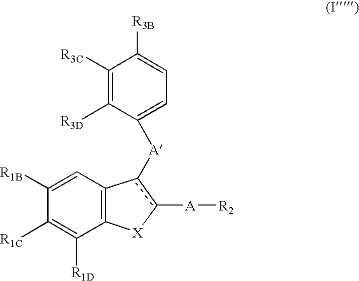
wherein X is O, S, NR (wherein R is hydrogen, sulfonyl, C₁₋₆alkyl, C₁₋₇acyl, or an aryl group) or C═O:
R_{1B}-R_{1D} and R_{3B}-R_{3D} are independently selected from hydrogen, hydroxy, methoxy, and amino or any 2 adjacent R₁ and/or R₃ groups from R_{1B-R}_{1D} and R_{3B}-R_{3D} form a dioxolanyl group;
R₂ is an optionally substituted aryl group;
A and A′ are independently selected from the group consisting of a single bond, C═O, CH₂, and CH(OR′), (R′ is hydrogen, C₁₋₆alkyl or C₁₋₇acyl); and
provided that the compound is not;
3-(3′,4′,5′-trimethoxybenzoyl)-2-(4′-methoxyphenyl)-6-methoxybenzo[b]thiophene;
3-(2′,6′-dimethoxybenzoyl-2-(4′-methoxyphenyl)-6-methoxybenzo[b]thiophene;
3-(3′,5′-dimethoxybenzoyl)-2-(4′-methoxyphenyl)-6-methoxybenzo[b]thiophene;
3-(3′,4′-dimethoxybenzoyl)-2-(4′-methoxyphenyl)-6-methoxybenzo[b]thiophene;
3-(4′-methoxybenzoyl)-2-(4′-methoxyphenyl)-6-methoxybenzo[b]thiophene;
3-(4′-ethoxybenzoyl)-2-(4′-methoxyphenyl)-6-methoxybenzo[b]thiophene;
3-(3′,4′,5′-triethoxybenzoyl)-2-(4′-methoxyphenyl)-6-methoxybenzo[b]thiophene;
3-[3′-(3′,4′,5′-trimethoxyphenyl)propionyl]-2-(4′-methoxyphenyl)-6-methoxybenzo[b]thiophene;
3-(3′, 4′,5′-triethoxybenzoyl)-2-(4′-ethoxyphenyl)-6-ethoxybenzo[b]thiophene;
3-(4′-ethoxy-3″,5′-dimethoxybenzoyl)-2-(4′-methoxyphenyl)-6-methoxybenzo[b]thiophene:
3-(4′-N,N-dimethylaminobenzoyl)-2-(4″-methoxyphenyl)-6-methoxybenzo[b]thiophene;
3-(3′,4′,5′-triflurobenzoyl)-2-(4′-methoxyphenyl)-6-methoxybenzo[b]thiophene;
3-(2′,3′,4′,5′,6′-pentafluorobenzoyl)-2-(4′-methoxyphenyl)-6-methoxybenzo[b]thiophene;
3-(3′, 4′,5′-trimethoxybenzoyl)-2-(4′-methoxyphenyl)benzo[b]thiophene;
3-(3′, 4′,5′-trimethoxybenzoyl)-2-(4′-ethoxyphenyl)-6-ethoxybenzo[b]thiophene;
3-(4′-hydroxy-3′, 5′-dimethoxybenzoyl)-2-(4′-methoxyphenyl)-6-methoxybenzo[b]thiophene:
2-(3″,4″, 5″-trimethoxybenzoyl)-3-(4′-methoxyphenyl)-6-methoxybenzo[b]furan;
2-(4′-methoxyphenyl)-3-(3′,3′,4′-trimethoxybenzoyl)-6-methoxyindole;
2-(3′-t-butylsiloxy-4′-methoxyphenyl)-3-(3′,4′,5′-trimethoxybenzoyl)-6-methoxyindole;
Disodium 2-(4′-methoxyphenyl-3-O-phosphate)-3-(3″,4″,5″-trimethoxybenzoyl)-6-methoxyindole;
2-(4′-methoxyphenyl)-3-(3″,4″,5″-trimethylbenzoyl)-4-methoxyindole;
Disodium 2-(3′-phosphormaidate-4′-methoxyphenyl)-3-(3-(3″,4″,5″-trimethoxybenzoyl)-6-methoxyindole;
2-(3′-hydroxy-4′-methoxyphenyl)-3-(3″,4″,5″-trimethoxybenzoyl)-4-methoxyindole;
2-(3′-amino-4′-methoxyphenyl)-3-(3″,4″ 5″-trimethoxybenzoyl)-4-methoxyindole;
Disodium 2-[(4′-methoxyphenyl)-3′-O-phosphate]-3-(3″,4″,5″-trimethoxybenzoyl)-4-methoxyindole;
2-(3′-diethylphosphoramidate-4′-methoxyphenyl)-3-(3″,4″,5″-trimethoxybenzoyl)-4-methoxyindole;
Disodium 2-(3′-phosphoramidate-4′-methoxyphenyl)-3-(3″,4″,5″-trimethoxybenzoyl)₄-methoxyindole;
2-(1-napth-1-yl)-3-(3″,4″,5″-trimethoxyphenyl)-5-methoxyindole;
2-(3′,4′-methylenedioxyphenyl)-3-(3″,4″,5″-trimethoxyphenyl)-5-methoxyindole;
2-(furan-2-yl)-3-(3′,4′,5′-trimethoxyphenyl)-5-methoxyindole;
2-(furan-3-yl)-3-(3′,4′,5′-trimethoxyphenyl)-5-methoxyindole;
2-(5-methylfuran-2-yl)-3-(3′,4′,5′-trimethoxyphenyl)-5-methoxyindole.

8. A compound of formula (I′″″) according to claim 7 wherein X is O, C═O or NR.

9. A compound of formula (I′″″) according to claim 8 wherein X is O.

10. A compound of formula (I′″″) according to claim 7 wherein X is O and A′ is C═O and A is a single bond.

11. A compound of formula (I′″″) according to claim 7 wherein 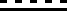
represents a double bond.

12. A compound of formula (IV) 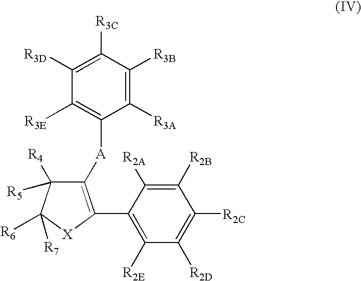
wherein X is O, S or NR″ (R″ is aryl, aroyl, acyl, benzyl, alkyl or sulphonyl)
A is a single bond C═O, CH(OR′) (R′ is hydrogen, C₁₋₆alkyl, C₁₋₇acyl), CH₂, O, S or NR(R is hydrogen, C₁₋₆ hydrogen, C₁₋₆alkyl or C₁₋₇acyl);
R_{2A}-R_{2E} and R_{3A}-R_{3E} are independently selected from hydrogen, hydroxy, optionally substituted alkoxy, optionally substituted alkyl, optionally substituted acyloxy, amino, optionally substituted alkylamino, optionally substituted dialkylamino, optionally substituted acylamino or where any two adjacent R₂ and/or R₃ group from R_{2A}-R_{2D} and R_{3A}-R_{3E} together form —O—CH₂—O—.
R₄-R₇ are independently selected from hydrogen, hydroxy, alkoxy, alkyl and amino.

13. A method of preparing a compound of formula (I′) 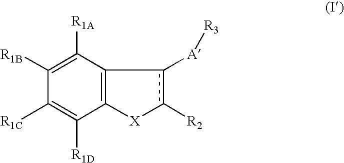
wherein
X is O, NH or NR, (wherein R is H, sulfonyl, C₁₋₆alkyl, C₁₋₇acyl or an aryl group)
A′is independently selected from a single bond, CH₂, C═O, and CH(OR′) (R′ is H, C₁₋₆alkyl or C₁₋₇acyl);
R_{1A}-R_{1D} are independently selected from hydrogen, hydroxy, optionally substituted alkoxy, optionally substituted alkyl, optionally substituted acyloxy, amino, optionally substituted alkylamino, optionally substituted dialkylamino, optionally substituted acylamino, or any 2 adjacent R_{1A}-R_{1D} together form —O—CH₂—O—;
R₂ and R₃ are optionally substituted aryl groups,
said method comprising the steps of:
a) coupling a compound of formula (1) with an alkyne of formula (2) in the presence of a nickel or palladium coupling agent 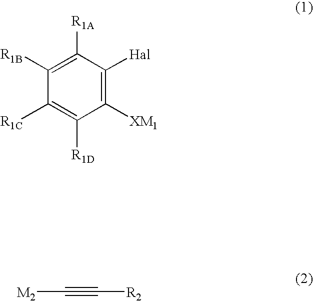
wherein
R_{1A}-R_{1D}, R₂ and X are as above:
Hal is I Br or Cl:
M₁ is a metal or a metal species thereof, said metal selected from the group consisting of Li, Na, K, Mg, Cs and Ba;
M₂ is a metal, or a metal species thereof, said metal selected from the group consisting of Mg, Zn, Cu, B, Si, Mn, Sn, Ge and Al;
X is O, NR(R is sulfonyl, C₁₋₆alkyl or C₁₋₇acyl);
e) reacting in situ the resulting coupled product, with R₃-L wherein R₃ is an optionally substituted aryl group, and wherein L is a leaving group, optionally in the presence of carbon monoxide; and
f) optionally reducing the resulting product, when A′ is C═O, to afford compounds in which A═CH₂ or CH(OR′).

14. A combinatorial library of compounds comprising at least two compounds of formula (I′) according to claim 13.

15. A method for preparing a compound of formula (I″): 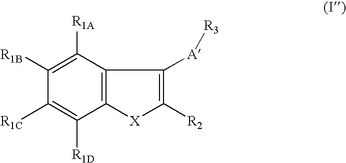
wherein
X is S
A′ is selected from a single bond, CH₂, C═O, and CH(OR′) (R′ is H, C₁₋₆alkyl or C₁₋₇acyl);
R_{1A}-R_{1D} are independently selected from hydrogen, hydroxy, optionally substituted alkoxy, optionally substituted alkyl, optionally substituted optionally substituted acyloxy, amino, optionally substituted alkylamino, optionally substituted dialkylamino, optionally substituted acylamino, or any two adjacent R_{1A}-R_{1D} together form —O—CH₂—O—;
R₂ and R₃ are optionally substituted aryl groups;
said method comprising the steps of:
a) coupling a compound of formula (3) with a compound of formula (4) in the presence of a nickel or palladium coupling agent 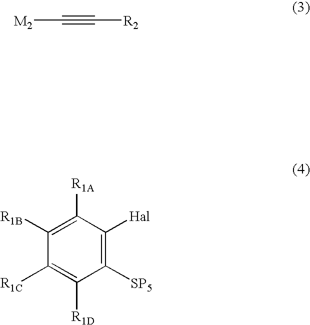
wherein
R_{1A}-R_{1D}, Hal, M₂ and R₂ are as above, and PS is a sulfur protecting group capable of stabilizing a positive charge;
b) cyclising the resulting coupled product in the presence of a Hal⁻ producing reagent to give (5) 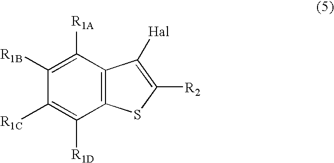
wherein
Hal is Cl, Br or I;
c) coupling (5) with either the moiety R₃—C(O)—or R₃—wherein R₃ is an optionally substituted aryl group, and
g) optionally reducing the coupled product when A′ is C═O, to afford compounds in which A′═CH₂ or CH(OR′).

16. A combinatorial library of compounds comprising at least two compounds of formula (I″) according to claim 15.

17. A method for preparing a compound of formula (I′″) 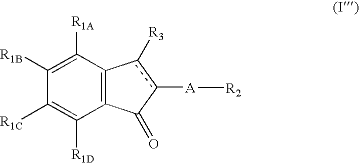
wherein
A is selected from a single bond, CH₂, C═O and CH(OR′) (R′ is H, C₁₋₆alkyl or C₁₋₇acyl);
R_{1A}-R_{1D} are independently selected from hydrogen, hydroxy, optionally substituted alkoxy, optionally substituted alkyl, optionally substituted acyloxy, amino, optionally substituted alkylamino, optionally substituted dialkylamino or any 2 adjacent R_{1A}-R_{1D} together form —O—CH₂—O; 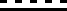
is an optional double bond:
R₂ and R₃ are optionally substituted aryl groups;
said method comprising the steps of:
(b) reacting compound (6) with compound (7): or reacting compound 6(a) with compound 7(a); 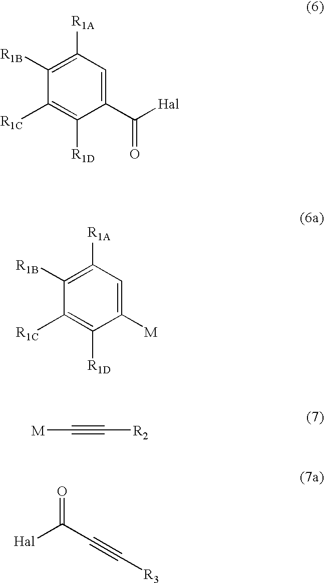
to form a compound (9) 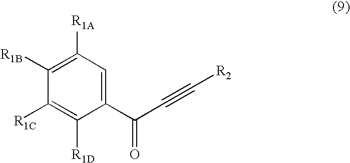
wherein
M is Li, Na, K or MgHal (Hal is Br, Cl or I);
b) treating compound (9) with a metal hydride in the presence of a palladium coupling agent;
c) coupling the resulting product with R₃-Hal or R₃—C(O)-Hal (wherein Hal is Cl, Br or I) to provide either compound (10) or (11); and 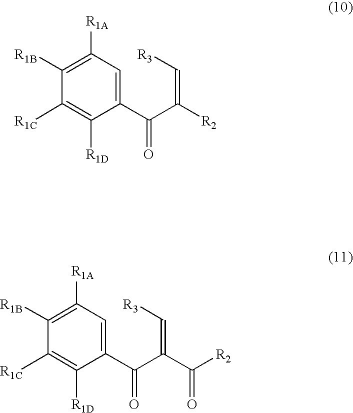
(d) cyclising (10) or (11) under acidic conditions to form an indanone and optionally treating the cyclised product With ma oxidising agent to form an indenone.

18. A combinatorial library of compounds comprising at least two compounds of formula (I′″) according to claim 17.

19. A method for preparing a compound of Formula (I″″) 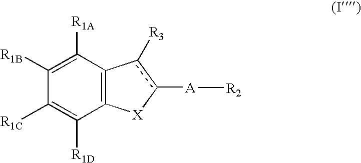
Wherein
X is O, S or NR (wherein R═H, C₁₋₆alkyl or C(O)C₁₋₆alkyl);
R_{1A}-R_{1D} are as defined in claim 1:
A is C═O, CH₂ or CH(OR′) (wherein R′ is H, C₁₋₆alkyl or C₁₋₇acyl); and
R₂ and R₃ are optionally substituted aryl groups;
comprising the steps of
a) coupling a compound (12) with compound (13) 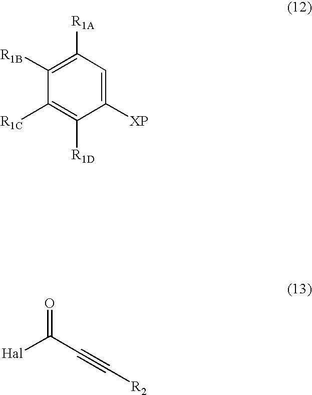
wherein Hal is Cl, Br, or I,
to form a compound of formula (14); 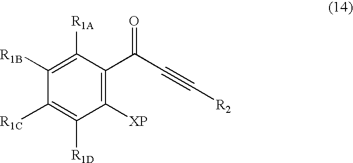
b) where X is S, protecting the thiol with a sulfur-protecting group
c) reacting (14) with a compound
M₁—R₃
wherein
M₁ is Li, Na, K, Mg, Cs or Ba, and R₃ is an optionally substituted aryl group; to form 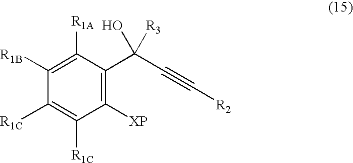
wherein
when X is O, then P is H and when X is S, P is a sulfur protecting group and when X is NR, R is a hydrogen, sulfonyl, C₁₋₆alkyl, C₁₋₇aryl, or an aryl group;
e) treating (15) with a Hal⁺ producing reagent, to afford cyclisation;
e) and optionally reducing the cyclised product when A′ is C═O, to afford libraries of compounds in which A′ is CH₂ or CH(OR′).

20. A combinatorial library of compounds comprising at least two compounds of formula (I″″) according to claim 19.

21. A compound of Formula II 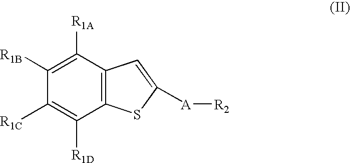
wherein
R_{1A}-R_{1D} are independently hydrogen, hydroxy, optionally substituted alkoxy, optionally substituted alkyl, optionally substituted optionally substituted acyloxy, amino, optionally substituted alkylamino, optionally substituted dialkylamino or 2 adjacent R_{1A}-R_{1D} are O—CH₂—O;
R₂ and R₃ are optionally substituted aryl groups; and
A is C═O, CH₂ or CH(OR′) (wherein R′═H, C₁₋₆alkyl or C₁₋₇acyl).

22. A combinatorial library of compounds comprising at least two compounds of formula 11 according to claim 21.

23. A compound of Formula (III) 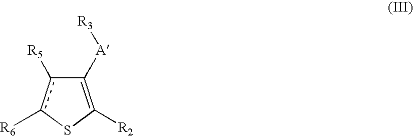
wherein
R₂ and R₃ are optionally substituted aryl groups;
A′ is CO, CH₂, CH(OR′) (wherein R′═H, C₁₋₆alkyl or C₁₋₇acyl) or a single bond;
R₅ and R₆ can independently be hydrogen, optionally substituted alkyl, optionally substituted aryl or optionally substituted alkenyl; 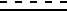
is an optional double bond.

24. A combinatorial library of compounds comprising at least two compounds of formula III according to claim 23.

25. A compound selected from the group consisting of:
6-methoxy-2-(4-methoxyphenyl)-3-3,4,5-trimethoxyphenyl)indole;
6-methoxy-2-(3-isopropoxy-4-methoxyphenyl)-3-(3,4,5-trimethoxyphenyl)indole;
6-methoxy-2-(3-hydroxy-4-methoxyphenyl)-3-(3,4,5-trimethoxyphenyl)indole;
6-methoxy-2-(3-isopropoxy-4-methoxyphenyl)-3-(3,4,5-trimethoxybenzoyl)indole;
6-methoxy-2-(3-hydroxy-4-methoxyphenyl)-3-(3,4,5-trimethoxybenzoyl)indole;
2,3-[2′-(3″,4″-methylenedioxyphenyl)-3-(3″,4″,5′″-trimethoxyphenyl)furano]-17-O-benzylestradiol;
6-methoxy-2-(4′-methoxyphenyl)-3-(3″,4″,5″-trimethoxybenzoyl)benzo[b]furan;
6-methoxy-2-(3-hydroxy-4-methoxyphenyl)-3-(3,4,5-trimethoxybenzoyl)benzo[b]furan;
6-methoxy-2-(4-methoxyphenyl)-3-[α-hydroxy-α-(3,4,5-trimethoxyphenyl)methyl]benzo[b]furan;
6-methoxy-2-(4-methoxyphenyl)-3-(3,4,5-trimethoxybenzyl)benzo[b]furan;
6-methoxy-2-(3-hydroxy-4-methoxyphenyl)-3-[α-hydroxy-α-(3,4,5-trimethoxyphenyl)methyl}benzo[b]furan;
3-(3′-isopropoxy-4′-methoxyphenyl)-4,5,6-trimethoxy-2-(3″,4″,5″-trimethoxybenzoyl)-1-indanone;
3-(3′-isopropoxy-4′-methoxyphenyl)-4,5,6-trimethoxy-2-(3″,4″,5″-trimethoxybenzoyl)indenone;
3-(3′-hydroxy-4′-methoxyphenyl)-4,5,6-trimethoxy-2-(3″,4″,5″-trimethoxybenzoyl)indenone;
(±)trans-3-(3′-isopropoxy-4′-methoxyphenyl)-4,5,6-trimethoxy-2-(3″,4″,5″-trimethoxyphenyl)-1-indanone;
(±)trans-3-(3′-isopropoxy-4′-methoxyphenyl)-4,5,6-trimethoxy-2-(3″,4″,5″-trimethoxyphenyl)-1-indenone;
2-(3′-acetoxy-4′-methoxyphenyl)-4,5-dihydro-3-(3″,4″,5″,-trimethoxyphenyl)thiophene;
2-(3′-acetoxy-3′-methoxyphenyl)-3-(3″,4″,5″,-trimethoxybenzoyl)thiophene;
2-(3′-isopropoxy-4′-methoxyphenyl)-6-methoxy-3-(3″,4″5″-trimethoxybenzoyl)benzo[b]thiophene;
2-(3′-hydroxy-4′-methoxyphenyl)-6-methoxy-3-(3″,4″,5″-trimethoxybenzoyl)benzo[b]thiophene;
2-(3′-hydroxy-4′-methoxyphenyl)-5,6-methylendioxy-3-(3″,4″-5″-trimethoxybenzoyl)benzo[b]thiophene;
3-(α-hydroxy-3′-hydroxy-4′-methoxybenzyl)-6-methoxy-3-(3″,4″5″-trimethoxybenzoyl)benzo[b]thiophene;
5-isopropoxy-2-(3′-isopropoxy-4′-methoxybenzoyl)-6-methoxybenzo[b]thiophene;
5-hydroxy-2-(3′-hydroxy-4′-methoxybenzoyl)-6-methoxybenzo[b]thiophene;
5-isopropoxy-2-(3′-isopropoxy-4′-methoxybenzoyl)-6-methoxy-3-(3″,4″,5″-trimethoxyphenyl)benzo[b]thiophene;
5-hydroxy-2-(3′-hydroxy-4′-methoxybenzoyl)-6-methoxy-3-(3″,4″,4″-trimethoxyphenyl)benzo[b]thiophene;
2-[α-(3″-isopropoxy-4″-methoxyphenyl)-3′,4′,5′-trimethoxybenzylidene]-5-isopropoxy-6-methoxybenzo[b]thiophen-3-one;
2-[2-oxo-1-(3′-isopropoxy-4′-methoxyphenyl)-2-(3″,4″ 5″-trimethoxyphenyl)-ethylidene]-5-isopropoxy-6-methoxybenzo[b]thiophen-3-one.

26-27. (canceled)

28. A method of treating a condition requiring an anti-mitotic agent including administering to a subject in need thereof a compound according to claim 25.

29. A method according to claim 28 wherein the condition to be treated is a tumour.

30. A composition comprising a compound according to claim 25 together with a pharmaceutically acceptable carrier.

31. A kit of components to prepare a library of compounds to be used for screening for TPI activity said kit comprising at least two substrates selected from (a), (b), and (c) as defined in claim 1.
